(19) United States
(12) Patent Application Publication
(10) Pub. No.: US 2020/0399431 A1
(43) Pub. Date: Dec. 24, 2020

(54) Covalently Cross-Linked Hyaluronic Acid Aerogel, Hydrogel, and Preparation Method Thereof

(71) Applicant: Hunan Elixer Therapeutics Co., Ltd., High-tech Industrial Development Zone (CN)

(72) Inventors: Yu Huang, Jinshi, Changde (CN); Bihuang Hu, Jinshi, Changde (CN)

(73) Assignee: Hunan Elixer Therapeutics Co., Ltd., Jinshi, Changde (CN)

(21) Appl. No.: 16/977,651

(22) PCT Filed: Jun. 21, 2019
(86) PCT No.: PCT/CN2019/092172

§ 371 Date: Sep. 2, 2020

(30) Foreign Application Priority Data
Jun. 27, 2018 (CN) 201810674081.1

(51) Int. Cl. C08J 3/075; C08J 3/24; A61L 26/00; A61L 24/08; A61L 24/00

(52) U.S. Cl. CPC C08J 3/075; C08J 3/24; A61L 26/0023; A61L 24/08; C08J 2305/08; A61L 24/0031; A61L 26/008; A61L 24/0042; A61L 26/009

(57) ABSTRACT

The present invention relates to a covalently cross-linked hyaluronic acid aerogel, a hydrogel, and a preparation method thereof. The method includes steps of: taking hyaluronic acid as a raw material to prepare a hyaluronic acid aqueous solution; taking 1,4-butanediol diglycidyl ether, ethylene glycol diglycidyl ether, or polyethylene glycol diglycidyl ether as a crosslinking agent; promoting a chemical crosslinking through covalent bonds between the crosslinking agent and the hyaluronic acid by a freeze-drying technique to obtain a covalently cross-linked hyaluronic acid aerogel; furthermore, soaking the covalently cross-linked hyaluronic acid aerogel in purified water, which absorbing water and swelling to obtain a covalently cross-linked hyaluronic acid hydrogel. The covalently cross-linked hyaluronic acid aerogel has good liquid absorption properties, and can quickly absorb water and swell to the covalently cross-linked hyaluronic acid hydrogel.

### TECHNICAL FIELD

The present invention relates to the field of tissue engineering and medical materials, and in particular, to a covalently cross-linked hyaluronic acid aerogel, a hydrogel, and a preparation method thereof.

### BACKGROUND

Hydrogel is of a cross-linked hydrophilic polymeric three-dimensional network structure, and an aerogel is the product with the unchanged polymeric three-dimensional network structure after the moisture of which being removed. Aerogel has properties which hydrogel does not have, such as high porosity, high specific surface area, low density, higher stability and convenience in transportation, storage and use.

Hydrogel that is formed through the physical interaction between macromolecular chains rather than covalent bond crosslinking is called physical hydrogel, which is characterized in that it is poor in physical properties and will be gradually dissolved as the amount of solvent increased; whereas chemical hydrogel is of a network structure that is formed through covalent bond crosslinking, and therefore cannot be dissolved in any organic solvent and aqueous solutions. These two types of hydrogels can be distinguished by dissolution experiments and rheological frequency sweep experiments.

With high water-swellability and good biocompatibility and water permeability, hydrogel shows a good application prospect in the fields of tissue engineering and medical materials, such as used in the carriers of sustained release and controlled release for drugs and tissue engineering. Meanwhile, with excellent biological properties and physical properties, hydrogel can also be applied as sealants and adhesives for cosmetic surgery, surgery and used as wound dressing to promote wound healing. Therefore, the research and development of covalently cross-linked hydrogel have drawn much attention of domestic and foreign scholars.

As a natural straight-chain anionic macromolecular polysaccharide, hyaluronic acid (HA) is a non-sulfated linear glycosaminoglycan, which is composed of N-acetyl-D-glucosamine and D-glucuronic acid disaccharide units, wherein monosaccharides are connected through β-1,3-glucosidic bonds, while the disaccharide units are connected through β-1,4-glucosidic bonds. Hyaluronic acid widely exists in human tissues and organs with characteristics of biodegradability, no immunity, no cytotoxicity, etc., but hyaluronic acid can be easily degraded by hyaluronidase in vivo. Covalently cross-linked hyaluronic acid hydrogel formed by chemical crosslinking can prolong its residence time in vivo, showing its superiority. In 2003, Restylane® products produced by Q-Med AB were approved by FDA of the U.S. to officially enter the U.S. Market as a class of hyaluronic acid facial injection filler; and in 2005, Restylane® became the first modified hyaluronic acid gel for injection approved by the CFDA (China Food and Drug Administration) in China. So far, the U.S. FDA has approved Restylane®, Hylaform®, Juvederm® and other hyaluronic acids to come into the market.

Meanwhile, there are also patents and literatures at home and abroad that disclose study results about chemically cross-linked hyaluronic acid hydrogels. The most commonly used crosslinking agents for the preparation of chemically cross-linked hyaluronic acid hydrogels are glycidyl ether, divinyl sulfone, carbodiimide, aldehydes, genipin, polyethylene glycol, etc. Among them, 1,4-butanediol diglycidyl ether (such as 1,4-butanediol diglycidyl ether used in U.S. Pat. No. 5,827,937) was used as a crosslinking agent to carry out crosslinking reaction with hyaluronic acid, but this reaction required 4 hours of activation at 40° C., and then diluted to 0.5%˜1%, and a vacuum distillation process in order to obtain hyaluronic acid hydrogel (dry gel). The preparation process of this method was complicated, and the reaction temperature was relatively high, which can easily result in the breakage of hyaluronic acid macromolecular chain. 1,4-butanediol diglycidyl ether was also used as a crosslinking agent to prepare hyaluronic acid hydrogel in WO8600079. In this reaction, crosslinking was carried out under the condition of 50° C., the reaction temperature was relatively high, which could easily result in the breakage and degradation of hyaluronic acid chain. In CN101502677, glycidyl ether and hyaluronic acid were mixed in sodium hydroxide solution, and the temperature of the mixture was kept under 40° C. to 80° C. for 2 to 8 hours, so that a water-insoluble hydrogel was prepared. In the reaction process, hyaluronic acid would be deteriorated due to hydrolysis of strong alkali in the solution, while the crosslinking agent BDDE had thermal instability under the alkaline condition, and pyrolyzed BDDE decomposed in high temperature had great impact on the biocompatibility of hydrogel. Moreover, the strong alkaline product required further neutralization before being used as a biocompatible material.

Hydrogels that were prepared using divinyl sulfone (as in patent CN102813961A), carbodiimide (as in patent CN1893989A) and aldehydes (as in patent CN101062017) as crosslinking agents respectively have nonuniform crosslinking effect, poor stability, nonuniform crosslinking degree and high glutaraldehyde toxicity, the biocompatibility of the prepared hyaluronic acid nanoparticles is not high, and adverse reaction, such as implant calcification, can easily occur.

At present, no covalently cross-linked hyaluronic acid aerogels and preparation methods thereof have been reported yet.

Although covalently cross-linked hydrogel has made tremendous progress in application in the fields of tissue engineering and medical materials, however, due to the defects of raw materials, the complexity of preparation processes and insufficient properties, its wide application in the field of tissue engineering and medical materials are limited. Therefore, the development of a covalently cross-linked hydrogel with excellent properties under mild conditions without using toxic reagents is still confronted with challenge.

### SUMMARY

In view of the defects existing in the above prior arts, the present disclosure aims at providing a novel covalently cross-linked hyaluronic acid aerogel to be directly used as a surgical dressing, or after being added with normal saline to become a covalently cross-linked hyaluronic acid hydrogel to be served as a surgical sealant, a tissue filler or a drug carrier. Crosslinking reaction would be carried out under a non-alkaline (in 18.2 Me ultrapure water) and freeze-drying environment to obtain the cross-linked hyaluronic acid aerogel and the cross-linked hyaluronic acid with excellent properties.

In order to realize the above purpose, the present disclosure adopts the following technical scheme:

(1) taking hyaluronic acid as a material, preparing hyaluronic acid aqueous solution using ultrapure water (18.2 Me), and then standing for defoaming;

(2) adding a crosslinking agent into the hyaluronic acid aqueous solution, sufficiently shaking to be mixed uniformly, and pouring the mixture into a culture dish and immediately freeze-dried, to obtain a covalently cross-linked hyaluronic acid aerogel;

(3) soaking the covalently cross-linked hyaluronic acid aerogel in a solvent, the covalently cross-linked hyaluronic acid aerogel would absorb water and swell, to obtain a covalently cross-linked hyaluronic acid hydrogel.

Preferably, the molecular weight of hyaluronic acid is 3.5×10⁵ to 1.5×10⁶ Da. Preferably, the concentration of the hyaluronic acid in the aforementioned hyaluronic acid aqueous solution is 0.5% to 4% (w/v).

Preferably, the aforementioned crosslinking agent is 1,4-butanediol diglycidyl ether (BDDE), as shown in formula 1.

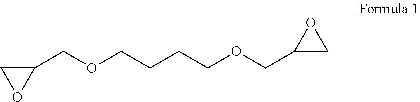 [SMILES: C(CCOCC1CO1)COCC1CO1]

Preferably, the aforementioned crosslinking agent is ethylene glycol diglycidyl ether (GDGE), as shown in formula 2.

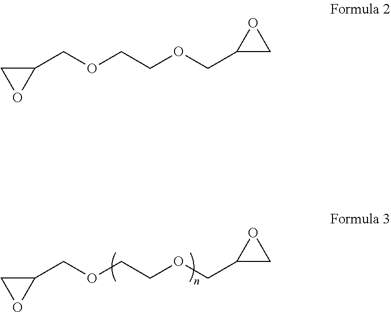

Preferably, the concentration of the aforementioned crosslinking agent in hyaluronic acid aqueous solution is 0.1% to 1% (v/v).

In the present invention, a crosslinking agent (BDDE, GDGE or PEG500) containing diglycidyl ether groups is chosen to carry out a freeze-drying experiment with three types of polysaccharides, i.e. chondroitin sulfate, alginic Acid and hyaluronic acid respectively. Surprisingly, it was discovered that chondroitin sulfate and alginic acid as two polysaccharides could not carry out chemical crosslinking reaction with the crosslinking agent containing diglycidyl ether groups under the freeze-drying condition to obtain the covalently cross-linked aerogel and the covalently cross-linked hydrogel, while hyaluronic acid could carry out chemical crosslinking reaction with the crosslinking agent containing diglycidyl ether groups under the freeze-drying condition to obtain the covalently cross-linked hyaluronic acid aerogel, and when soaked in ultrapure water or normal saline, the covalently cross-linked hyaluronic acid aerogel could absorb water and swell to form the covalently cross-linked hyaluronic acid hydrogel.

Compared with the prior art, the present invention has the following prominent technical effects:

The present invention employs the freeze-drying technique for the first time to promote the covalent crosslinking between the crosslinking agent containing a diglycidyl ether and the hyaluronic acid under the neural condition, while the same reaction system could not form the covalently cross-linked hydrogel after standing for 72 hours under normal temperature (28° C.). Wherein, an ethylene oxide group in the diglycidyl ether carries out ring-opening reaction with the hydroxy group on hyaluronic acid under freeze-drying and neural conditions to form the covalently cross-linked hyaluronic acid aerogel by generating an ether bond. Such aerogel can be served as a novel surgical dressing for clinical use directly. The covalently cross-linked hyaluronic acid aerogel is soaked in purified water, absorbing water and swelling, to obtain the covalently cross-linked hyaluronic acid hydrogel. The crosslinking agent used by the method of the present invention is relatively safe (such crosslinking agent has been used in products of the same category that have been approved by FDA of the U.S. to come into the market), the preparation process is simple and easy to implement, and the production cost is low.

The covalently cross-linked hyaluronic acid aerogel and the covalently cross-linked hyaluronic acid hydrogel prepared by the present invention can retard the degradation of hyaluronic acid, prolonging its residence time in vivo. The covalently cross-linked aerogel obtained by freeze-drying step can be directly applied as a surgical dressing, and is easier to store and more stable under normal temperature due to the removal of hydrosolvent. Therefore, the obtained hydrogel can be used as a cytoskeleton to provide a place and a suitable condition for physiological activities (proliferation and directional differentiation) of cells; moreover, the hydrogel can also create a wet closed environment for the healing of a wound, so that the wound can healed more quickly. The covalently cross-linked hyaluronic acid hydrogel prepared by the present invention has good biocompatibility and stable structure, which is significant for the development of cytoskeletons and medical wound dressings with stable structure, excellent properties and good biocompatibility.

Because the cross-linked hyaluronic acid of the present invention react under a neutral condition which does not need to add with strong alkaline substances (such as NaOH, etc.), after the reaction being completed, the step of removing the alkaline substance is not needed. Consequently, the operating steps are reduced, and the production cost is lowered. Moreover, adverse reaction brought to subsequent implantation in the human body by the residues of NaOH and other substances is avoided.

### BRIEF DESCRIPTION OF THE DRAWINGS

FIG. 1 is the morphologies of the covalently cross-linked hyaluronic acid hydrogels prepared in examples 9, 10, 11 and 12.

FIG. 2 is the morphologies of the covalently cross-linked hyaluronic acid hydrogels prepared in examples 45, 46, 47 and 48.

FIG. 3 is the degradation times of the covalently cross-linked hyaluronic acid hydrogels prepared in examples 1-12 in PBS buffer solution.

FIG. 4 is the degradation times of the covalently cross-linked hyaluronic acid hydrogels prepared in examples 13-24 in PBS buffer solution.

FIG. 5 is the time sweep of a “4.0% HA³⁵+0.1% BDDE” sample.

FIG. 6 is the frequency sweep of the covalently cross-linked hyaluronic acid hydrogel prepared in example 9.

FIG. 7 is the strain sweep of the covalently cross-linked hyaluronic acid hydrogel prepared in example 9.

FIG. 8 is the time sweep of a “4.0% HA³⁵+0.2% BDDE” sample.

FIG. 9 is the frequency sweep of the covalently cross-linked hyaluronic acid hydrogel prepared in example 10.

FIG. 10 is the strain sweep of the covalently cross-linked hyaluronic acid hydrogel prepared in example 10.

FIG. 11 is the time sweep of a “4.0% HA³⁵+0.4% BDDE” sample.

FIG. 12 is the frequency sweep of the covalently cross-linked hyaluronic acid hydrogel prepared in example 11.

FIG. 13 is the strain sweep of the covalently cross-linked hyaluronic acid hydrogel prepared in example 11.

FIG. 14 is the time sweep of a “4.0% HA³⁵+1.0% BDDE” sample.

FIG. 15 is the frequency sweep of the covalently cross-linked hyaluronic acid hydrogel prepared in example 12.

FIG. 16 is the strain sweep of the covalently cross-linked hyaluronic acid hydrogel prepared in example 12.

FIG. 17 is the time sweep of a “4.0% HA³⁵+0.1% GDGE” sample.

FIG. 18 is the frequency sweep of the covalently cross-linked hyaluronic acid hydrogel prepared in example 21.

FIG. 19 is the strain sweep of the covalently cross-linked hyaluronic acid hydrogel prepared in example 21.

FIG. 20 is the time sweep of a “4.0% HA³⁵+0.2% GDGE” sample.

FIG. 21 is the frequency sweep of the covalently cross-linked hyaluronic acid hydrogel prepared in example 22.

FIG. 22 is the strain sweep of the covalently cross-linked hyaluronic acid hydrogel prepared in example 22.

FIG. 23 is the time sweep of a “4.0% HA³⁵+0.4% GDGE” sample.

FIG. 24 is the frequency sweep of the covalently cross-linked hyaluronic acid hydrogel prepared in example 23.

FIG. 25 is the strain sweep of the covalently cross-linked hyaluronic acid hydrogel prepared in example 23.

FIG. 26 is the time sweep of a “4.0% HA³⁵+1.0% GDGE” sample.

FIG. 27 is the frequency sweep of the covalently cross-linked hyaluronic acid hydrogel prepared in example 24.

FIG. 28 is the strain sweep of the covalently cross-linked hyaluronic acid hydrogel prepared in example 24.

FIG. 29 is the time sweep of a “4.0% HA³⁵+0.1% PEG500” sample.

FIG. 30 is the frequency sweep of a covalently cross-linked hyaluronic acid hydrogel prepared in example 33.

FIG. 31 is the strain sweep of the covalently cross-linked hyaluronic acid hydrogel prepared in example 33.

FIG. 32 is the time sweep of a “4.0% HA³⁵+0.2% PEG500” sample.

FIG. 33 is the frequency sweep of a covalently cross-linked hyaluronic acid hydrogel prepared in example 34.

FIG. 34 is the strain sweep of the covalently cross-linked hyaluronic acid hydrogel prepared in example 34.

FIG. 35 is the time sweep of a “4.0% HA³⁵+0.4% PEG500” sample.

FIG. 36 is the frequency sweep of a covalently cross-linked hyaluronic acid hydrogel prepared in example 35.

FIG. 37 is the strain sweep of the covalently cross-linked hyaluronic acid hydrogel prepared in example 35.

FIG. 38 is the time sweep of a “4.0% HA³⁵+1.0% PEG500” sample.

FIG. 39 is the frequency sweep of a covalently cross-linked hyaluronic acid hydrogel prepared in example 36.

FIG. 40 is the strain sweep of the covalently cross-linked hyaluronic acid hydrogel prepared in example 36.

FIG. 41 is the time sweep of a “1.5% HA¹⁵⁰+0.1% BDDE” sample.

FIG. 42 is the frequency sweep of a covalently cross-linked hyaluronic acid hydrogel prepared in example 45.

FIG. 43 is the frequency sweep of a “1.5% HA¹⁵⁰+0.1% BDDE” sample.

FIG. 44 is the strain sweep of the covalently cross-linked hyaluronic acid hydrogel prepared in example 45.

FIG. 45 is the time sweep of a “1.5% HA¹⁵⁰+0.2% BDDE” sample.

FIG. 46 is the frequency sweep of a covalently cross-linked hyaluronic acid hydrogel prepared in example 46.

FIG. 47 is the frequency sweep of a “1.5% HA¹⁵⁰+0.2% BDDE” sample.

FIG. 48 is the strain sweep of the covalently cross-linked hyaluronic acid hydrogel prepared in example 46.

FIG. 49 is the time sweep of a “1.5% HA¹⁵⁰+0.4% BDDE” sample.

FIG. 50 is the frequency sweep of a covalently cross-linked hyaluronic acid hydrogel prepared in example 47.

FIG. 51 is the frequency sweep of a “1.5% HA¹⁵⁰+0.4% BDDE” sample.

FIG. 52 is the strain sweep of the covalently cross-linked hyaluronic acid hydrogel prepared in example 47.

FIG. 53 is the time sweep of a “1.5% HA¹⁵⁰+1.0% BDDE” sample.

FIG. 54 is the frequency sweep of a covalently cross-linked hyaluronic acid hydrogel prepared in example 48.

FIG. 55 is the frequency sweep of a “1.5% HA¹⁵⁰+1.0% BDDE” sample.

FIG. 56 is the strain sweep of the covalently cross-linked hyaluronic acid hydrogel prepared in example 48.

FIG. 57 is the time sweep of a “1.5% HA¹⁵⁰+0.1% GDGE” sample.

FIG. 58 is the frequency sweep of a covalently cross-linked hyaluronic acid hydrogel prepared in example 57.

FIG. 59 is the frequency sweep of a “1.5% HA¹⁵⁰+0.1% GDGE” sample.

FIG. 60 is the strain sweep of the covalently cross-linked hyaluronic acid hydrogel prepared in example 57.

FIG. 61 is the time sweep of a “1.5% HA¹⁵⁰+0.2% GDGE” sample.

FIG. 62 is the frequency sweep of a covalently cross-linked hyaluronic acid hydrogel prepared in example 58.

FIG. 63 is the frequency sweep of a “1.5% HA¹⁵⁰+0.2% GDGE” sample.

FIG. 64 is the strain sweep of the covalently cross-linked hyaluronic acid hydrogel prepared in example 58.

FIG. 65 is the time sweep of a “1.5% HA¹⁵⁰+0.4% GDGE” sample.

FIG. 66 is the frequency sweep of a covalently cross-linked hyaluronic acid hydrogel prepared in example 59.

FIG. 67 is the frequency sweep of a “1.5% HA¹⁵⁰+0.4% GDGE” sample.

FIG. 68 is the strain sweep of the covalently cross-linked hyaluronic acid hydrogel prepared in example 59.

FIG. 69 is the time sweep of a “1.5% HA¹⁵⁰+1.0% GDGE” sample.

FIG. 70 is the frequency sweep of a covalently cross-linked hyaluronic acid hydrogel prepared in example 60.

FIG. 71 is the frequency sweep of a “1.5% HA¹⁵⁰+1.0% GDGE” sample.

FIG. 72 is the strain sweep of the covalently cross-linked hyaluronic acid hydrogel prepared in example 60.

FIG. 73 is the time sweep of a “1.5% HA¹⁵⁰+0.1% PEG500” sample.

FIG. 74 is the frequency sweep of a covalently cross-linked hyaluronic acid hydrogel prepared in example 69.

FIG. 75 is the frequency sweep of a “1.5% HA¹⁵⁰+0.1% PEG500” sample.

FIG. 76 is the strain sweep of the covalently cross-linked hyaluronic acid hydrogel prepared in example 69.

FIG. 77 is the time sweep of a “1.5% HA¹⁵⁰+0.2% PEG500” sample.

FIG. 78 is the frequency sweep of a covalently cross-linked hyaluronic acid hydrogel prepared in example 70.

FIG. 79 is the frequency sweep of a “1.5% HA¹⁵⁰+0.2% PEG500” sample.

FIG. 80 is the strain sweep of the covalently cross-linked hyaluronic acid hydrogel prepared in example 70.

FIG. 81 is the time sweep of a “1.5% HA¹⁵⁰+0.4% PEG500” sample.

FIG. 82 is the frequency sweep of a covalently cross-linked hyaluronic acid hydrogel prepared in example 71.

FIG. 83 is the frequency sweep of a “1.5% HA¹⁵⁰+0.4% PEG500” sample.

FIG. 84 is the strain sweep of the covalently cross-linked hyaluronic acid hydrogel prepared in example 71.

FIG. 85 is the time sweep of a “1.5% HA¹⁵⁰+1.0% PEG500” sample.

FIG. 86 is the frequency sweep of a covalently cross-linked hyaluronic acid hydrogel prepared in example 72.

FIG. 87 is the frequency sweep of a “5% HA¹⁵⁰+1.0% PEG500” sample.

FIG. 88 is the strain sweep of the covalently cross-linked hyaluronic acid hydrogel prepared in example 72.

FIG. 89 is the degradation time of the covalently cross-linked hyaluronic acid hydrogels in plasma.

FIG. 90 is a D-glucuronic acid standard solution and a regression equation.

FIG. 91 is the enzymatic degradation times of the covalently cross-linked hyaluronic acid hydrogels in vitro (the molecular weight of hyaluronic acid: 3.5×10⁵ Da, the concentration of hyaluronic acid: 4% (w/v)).

FIG. 92 is the enzymatic degradation times of the covalently cross-linked hyaluronic acid hydrogels in vitro (the molecular weight of hyaluronic acid: 1.5×10⁶ Da, the concentration of hyaluronic acid: 1.5% (w/v)).

FIG. 93 is the enzymatic degradation rates of the covalently cross-linked hyaluronic acid hydrogels in vitro (the molecular weight of hyaluronic acid: 3.5×10⁵ Da, the concentration of hyaluronic acid: 4% (w/v), crosslinking agent: BDDE).

FIG. 94 is the enzymatic degradation rates of the covalently cross-linked hyaluronic acid hydrogels in vitro (the molecular weight of hyaluronic acid: 3.5×10⁵ Da, the concentration of hyaluronic acid: 4% (w/v), crosslinking agent: GDGE).

FIG. 95 is the enzymatic degradation rates of the covalently cross-linked hyaluronic acid hydrogels in vitro (the molecular weight of hyaluronic acid: 3.5×10⁵ Da, the concentration of hyaluronic acid: 4% (w/v), crosslinking agent: PEG500).

FIG. 96 is the enzymatic degradation rates of the covalently cross-linked hyaluronic acid hydrogels in vitro (the molecular weight of hyaluronic acid: 1.5×10⁶ Da, the concentration of hyaluronic acid: 1.5% (w/v), crosslinking agent: BDDE).

FIG. 97 is the enzymatic degradation rates of the covalently cross-linked hyaluronic acid hydrogels in vitro (the molecular weight of hyaluronic acid: 1.5×10⁶ Da, the concentration of hyaluronic acid: 1.5% (w/v), crosslinking agent: GDGE).

FIG. 98 is the enzymatic degradation rates of the covalently cross-linked hyaluronic acid hydrogels in vitro (the molecular weight of hyaluronic acid: 1.5×10⁶ Da, and the concentration of hyaluronic acid: 1.5% (w/v), crosslinking agent: PEG500).

FIG. 99 is the relative growth rates of the HA hydrogel with 2 days of culture (Mr (HA)=1.5×10⁶ Da).

FIG. 100 is the relative growth rates of the HA hydrogel with 2 days of culture (Mr (HA)=3.5×10⁵ Da).

FIG. 101 is the cytofluorescent image of an “HA³⁵-P500-4-3” sample (2 days of culture).

FIG. 102 is the cytofluorescent image of an “HA¹⁵⁰-P500-4-3” sample (2 days of culture).

FIG. 103 is the growth of L929 cells on the “HA³⁵-P500-4-3” sample (2 days of culture).

FIG. 104 is the growth of L929 cells on the “HA¹⁵⁰-P500-3-3” sample (2 days of culture).

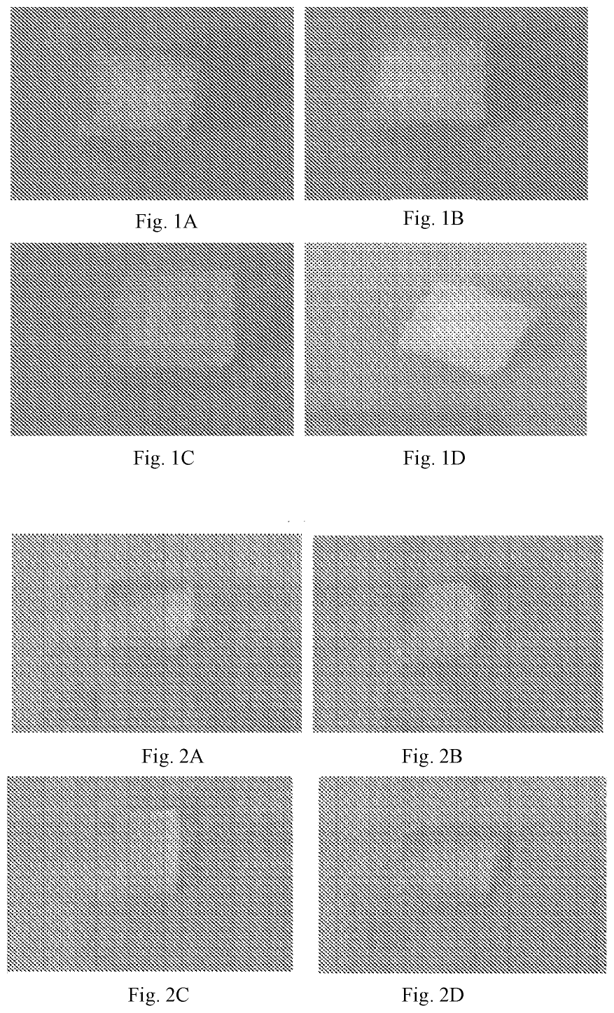

### DETAILED DESCRIPTION

In order to make the objective, technical scheme and beneficial effects of the present invention clearer and more obvious, the examples in the present invention will be described in detail below.

### Example 1

Hyaluronic acid (the molecular weight of hyaluronic acid: 3.5×10⁵ Da) (0.25 g) was weighed and dissolved in ultrapure water (25 mL) to obtain 1% hyaluronic acid aqueous solution (w/v) (pH7.0), which was shaken in a shaker under room temperature overnight for complete dissolution, and stood for defoaming; the solution was added with 1,4-butanediol diglycidyl ether (0.025 mL, the concentration of crosslinking agent: 0.1% (v/v)) and uniformly mixed, and the mixture was then poured into a culture dish with a diameter of 7.0 cm and freeze-dried immediately. In the step of freeze-drying, the culture dish was first put into a freeze dryer for prefreezing to −40° C., and was then temperature-programmed under a certain vacuum degree while the frozen state of the sample was kept to obtain a cross-linked aerogel (72 hours in total); the covalently cross-linked hyaluronic acid aerogel was soaked in buffer solution or purified water, absorbing water and swelling, to obtain a covalently cross-linked hyaluronic acid hydrogel.

As a control, during freeze-drying, the same hyaluronic acid and crosslinking agent solution were left at room temperature for the same time as the time of freeze-dried, and the sample was still a flowing solution.

Using the same conditions and steps as in example 1, chondroitin sulfate and alginic acid solutions were respectively freeze-dried with the crosslinking agent to obtain solid substances, which could be easily dissolved in water, indicating that a covalently cross-linked structure could not be formed.

### Example 2-72

The steps of examples 2-72 are the same as those of example 1, but the molecular weights and concentrations of hyaluronic acids, as well as the types and concentrations of crosslinking agents are different from those of example 1. The reaction parameters of examples 1-72 are shown in Table 1 below.

TABLE 1
Reaction Parameters

| | | Molecular | Concentration | | Concentration |
|---|---|---|---|---|---|
| | | Weight of | of Hyaluronic | | of Crosslinking |
| | | Hyaluronic | Acid | Crosslinking | Agent |
| Example | Sample Name | Acid/Da | (w/v, %) | Agent | (v/v, %) |
| 1 | HA³⁵-B-1-1 | 3.5 × 10⁵ | 1.0 | BDDE | 0.1 |
| 2 | HA³⁵-B-1-2 | 3.5 × 10⁵ | 1.0 | BDDE | 0.2 |
| 3 | HA³⁵-B-1-3 | 3.5 × 10⁵ | 1.0 | BDDE | 0.4 |
| 4 | HA³⁵-B-1-4 | 3.5 × 10⁵ | 1.0 | BDDE | 1.0 |
| 5 | HA³⁵-B-2-1 | 3.5 × 10⁵ | 2.0 | BDDE | 0.1 |
| 6 | HA³⁵-B-2-2 | 3.5 × 10⁵ | 2.0 | BDDE | 0.2 |
| 7 | HA³⁵-B-2-3 | 3.5 × 10⁵ | 2.0 | BDDE | 0.4 |
| 8 | HA³⁵-B-2-4 | 3.5 × 10⁵ | 2.0 | BDDE | 1.0 |
| 9 | HA³⁵-B-4-1 | 3.5 × 10⁵ | 4.0 | BDDE | 0.1 |
| 10 | HA³⁵-B-4-2 | 3.5 × 10⁵ | 4.0 | BDDE | 0.2 |
| 11 | HA³⁵-B-4-3 | 3.5 × 10⁵ | 4.0 | BDDE | 0.4 |
| 12 | HA³⁵-B-4-4 | 3.5 × 10⁵ | 4.0 | BDDE | 1.0 |
| 13 | HA35-G-1-1 | 3.5 × 10⁵ | 1.0 | GDGE | 0.1 |
| 14 | HA³⁵-G-1-2 | 3.5 × 10⁵ | 1.0 | GDGE | 0.2 |
| 15 | HA³⁵-G-1-3 | 3.5 × 10⁵ | 1.0 | GDGE | 0.4 |
| 16 | HA³⁵-G-1-4 | 3.5 × 10⁵ | 1.0 | GDGE | 1.0 |
| 17 | HA³⁵-G-2-1 | 3.5 × 10⁵ | 2.0 | GDGE | 0.1 |
| 18 | HA³⁵-G-2-2 | 3.5 × 10⁵ | 2.0 | GDGE | 0.2 |
| 19 | HA³⁵-G-2-3 | 3.5 × 10⁵ | 2.0 | GDGE | 0.4 |
| 20 | HA³⁵-G-2-4 | 3.5 × 10⁵ | 2.0 | GDGE | 1.0 |
| 21 | HA³⁵-G-4-1 | 3.5 × 10⁵ | 4.0 | GDGE | 0.1 |
| 22 | HA³⁵-G-4-2 | 3.5 × 10⁵ | 4.0 | GDGE | 0.2 |
| 23 | HA³⁵-G-4-3 | 3.5 × 10⁵ | 4.0 | GDGE | 0.4 |
| 24 | HA³⁵-G-4-4 | 3.5 × 10⁵ | 4.0 | GDGE | 1.0 |
| 25 | HA³⁵-P500-1-1 | 3.5 × 10⁵ | 1.0 | PEG500 | 0.1 |
| 26 | HA³⁵-P500-1-2 | 3.5 × 10⁵ | 1.0 | PEG500 | 0.2 |
| 27 | HA³⁵-P500-1-3 | 3.5 × 10⁵ | 1.0 | PEG500 | 0.4 |
| 28 | HA³⁵-P500-1-4 | 3.5 × 10⁵ | 1.0 | PEG500 | 1.0 |
| 29 | HA³⁵-P500-2-1 | 3.5 × 10⁵ | 2.0 | PEG500 | 0.1 |
| 30 | HA³⁵-P500-2-2 | 3.5 × 10⁵ | 2.0 | PEG500 | 0.2 |
| 31 | HA³⁵-P500-2-3 | 3.5 × 10⁵ | 2.0 | PEG500 | 0.4 |
| 32 | HA³⁵-P500-2-4 | 3.5 × 10⁵ | 2.0 | PEG500 | 1.0 |
| 33 | HA³⁵-P500-4-1 | 3.5 × 10⁵ | 4.0 | PEG500 | 0.1 |
| 34 | HA³⁵-P500-4-2 | 3.5 × 10⁵ | 4.0 | PEG500 | 0.2 |
| 35 | HA³⁵-P500-4-3 | 3.5 × 10⁵ | 4.0 | PEG500 | 0.4 |
| 36 | HA³⁵-P500-4-4 | 3.5 × 10⁵ | 4.0 | PEG500 | 1.0 |
| 37 | HA¹⁵⁰-B-1-1 | 1.5 × 10⁶ | 0.5 | BDDE | 0.1 |
| 38 | HA¹⁵⁰-B-1-2 | 1.5 × 10⁶ | 0.5 | BDDE | 0.2 |
| 39 | HA¹⁵⁰-B-1-3 | 1.5 × 10⁶ | 0.5 | BDDE | 0.4 |
| 40 | HA¹⁵⁰-B-1-4 | 1.5 × 10⁶ | 0.5 | BDDE | 1.0 |
| 41 | HA¹⁵⁰-B-2-1 | 1.5 × 10⁶ | 1.0 | BDDE | 0.1 |
| 42 | HA¹⁵⁰-B-2-2 | 1.5 × 10⁶ | 1.0 | BDDE | 0.2 |
| 43 | HA¹⁵⁰-B-2-3 | 1.5 × 10⁶ | 1.0 | BDDE | 0.4 |
| 44 | HA¹⁵⁰-B-2-4 | 1.5 × 10⁶ | 1.0 | BDDE | 1.0 |
| 45 | HA¹⁵⁰-B-3-1 | 1.5 × 10⁶ | 1.5 | BDDE | 0.1 |
| 46 | HA¹⁵⁰-B-3-2 | 1.5 × 10⁶ | 1.5 | BDDE | 0.2 |
| 47 | HA¹⁵⁰-B-3-3 | 1.5 × 10⁶ | 1.5 | BDDE | 0.4 |
| 48 | HA¹⁵⁰-B-3-4 | 1.5 × 10⁶ | 1.5 | BDDE | 1.0 |
| 49 | HA¹⁵⁰-G-1-1 | 1.5 × 10⁶ | 0.5 | GDGE | 0.1 |
| 50 | HA¹⁵⁰-G-1-2 | 1.5 × 10⁶ | 0.5 | GDGE | 0.2 |
| 51 | HA¹⁵⁰-G-1-3 | 1.5 × 10⁶ | 0.5 | GDGE | 0.4 |
| 52 | HA¹⁵⁰-G-1-4 | 1.5 × 10⁶ | 0.5 | GDGE | 1.0 |
| 53 | HA¹⁵⁰-G-2-1 | 1.5 × 10⁶ | 1.0 | GDGE | 0.1 |
| 54 | HA¹⁵⁰-G-2-2 | 1.5 × 10⁶ | 1.0 | GDGE | 0.2 |
| 55 | HA¹⁵⁰-G-2-3 | 1.5 × 10⁶ | 1.0 | GDGE | 0.4 |
| 56 | HA¹⁵⁰-G-2-4 | 1.5 × 10⁶ | 1.0 | GDGE | 1.0 |
| 57 | HA¹⁵⁰-G-3-1 | 1.5 × 10⁶ | 1.5 | GDGE | 0.1 |
| 58 | HA¹⁵⁰-G-3-2 | 1.5 × 10⁶ | 1.5 | GDGE | 0.2 |
| 59 | HA¹⁵⁰-G-3-3 | 1.5 × 10⁶ | 1.5 | GDGE | 0.4 |
| 60 | HA¹⁵⁰-G-3-4 | 1.5 × 10⁶ | 1.5 | GDGE | 1.0 |
| 61 | HA¹⁵⁰-P500-1-1 | 1.5 × 10⁶ | 0.5 | PEG500 | 0.1 |
| 62 | HA¹⁵⁰-P500-1-2 | 1.5 × 10⁶ | 0.5 | PEG500 | 0.2 |
| 63 | HA¹⁵⁰-P500-1-3 | 1.5 × 10⁶ | 0.5 | PEG500 | 0.4 |
| 64 | HA¹⁵⁰-P500-1-4 | 1.5 × 10⁶ | 0.5 | PEG500 | 1.0 |
| 65 | HA¹⁵⁰-P500-2-1 | 1.5 × 10⁶ | 1.0 | PEG500 | 0.1 |
| 66 | HA¹⁵⁰-P500-2-2 | 1.5 × 10⁶ | 1.0 | PEG500 | 0.2 |
| 67 | HA¹⁵⁰-P500-2-3 | 1.5 × 10⁶ | 1.0 | PEG500 | 0.4 |
| 68 | HA¹⁵⁰-P500-2-4 | 1.5 × 10⁶ | 1.0 | PEG500 | 1.0 |
| 69 | HA¹⁵⁰-P500-3-1 | 1.5 × 10⁶ | 1.5 | PEG500 | 0.1 |
| 70 | HA¹⁵⁰-P500-3-2 | 1.5 × 10⁶ | 1.5 | PEG500 | 0.2 |
| 71 | HA¹⁵⁰-P500-3-3 | 1.5 × 10⁶ | 1.5 | PEG500 | 0.4 |
| 72 | HA¹⁵⁰-P500-3-4 | 1.5 × 10⁶ | 1.5 | PEG500 | 1.0 |

According to the formulas of hyaluronic acids and crosslinking agents, as well as reaction systems of examples 2-72 in Table 1, a comparative experiment of (a) 72 hours of freeze-drying and (b) 72 hours of standing under room temperature was performed, and the results show that 72 hours of standing under room temperature could not obtain covalently cross-linked products.

### Example 73-Experiment on Degradation of Hyaluronic Acid Hydrogels in PBS Buffer Solution

By studying the stabilities of the covalently cross-linked hyaluronic acid hydrogels in PBS buffer solution, the obtained covalently cross-linked hyaluronic acid hydrogels were preliminarily screened.

1. The covalently cross-linked hyaluronic acid aerogels prepared in examples 1-72 were taken and respectively cut into the size of 0.5 cm×0.5 cm.

2. Each covalently cross-linked hyaluronic acid aerogel was soaked in 3 mL of PBS buffer solution, so that the covalently cross-linked hyaluronic acid aerogel could absorb liquid and swell, to obtain the covalently cross-linked hyaluronic acid hydrogel.

3. Under room temperature, an experiment on degradation in PBS buffer solution was performed, in which the covalently cross-linked hyaluronic acid hydrogel was put into the PBS buffer solution and weighed once every other day until the hydrogel was completely dissolved, and the PBS buffer solution was replaced once every other day. The experiment was repeated three times.

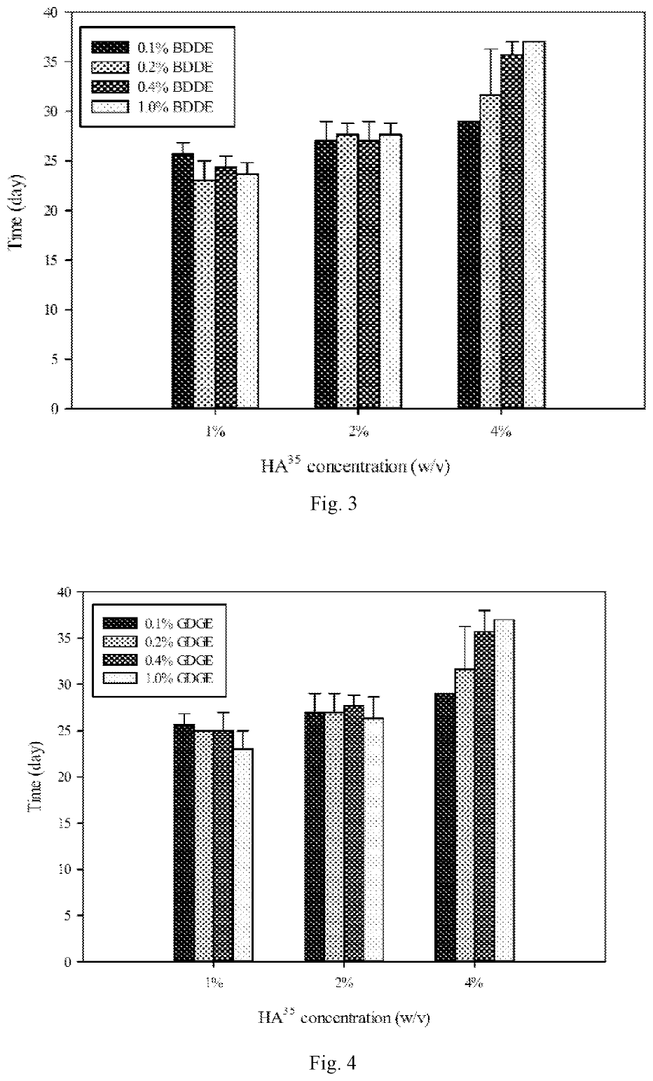

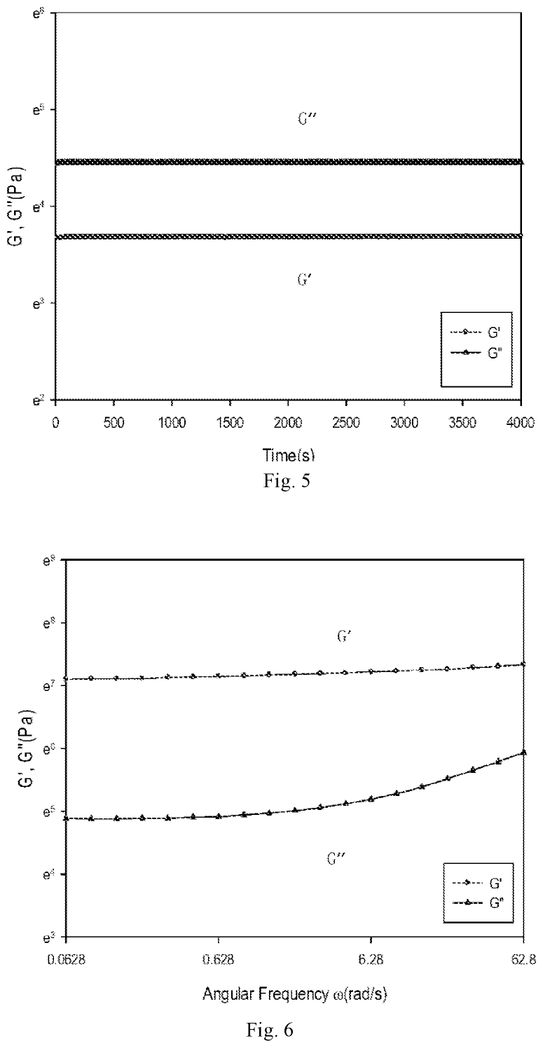

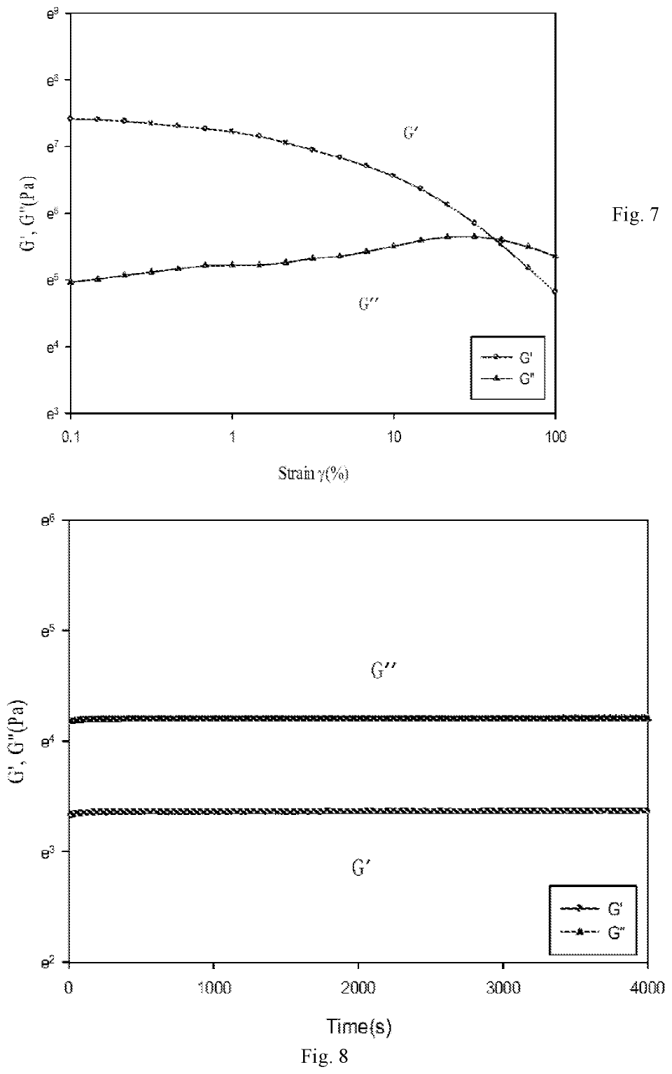

The experimental results are shown in FIG. 3-FIG. 8. No matter whether the molecular weight of hyaluronic acid was 3.5×10⁵ Da or 1.5×10⁶ Da, when the concentration of hyaluronic acid was increased, the degradation resistance of the hydrogels became higher. Wherein, hyaluronic acid with the molecular weight of 3.5×10⁵ Da carried out chemical crosslinking reaction with different crosslinking agents at different crosslinking agent doses respectively under the freeze-drying condition to obtain the covalently cross-linked hyaluronic acid aerogels. The covalently cross-linked hyaluronic acid aerogels were soaked in PBS, so that the covalently cross-linked hyaluronic acid aerogels could absorb liquid and swelling, to obtain the covalently cross-linked hyaluronic acid hydrogels. The degradation rates of the hydrogels were: 1.0% HA>2.0% HA>4.0% HA. In addition, hyaluronic acid with the molecular weight of 1.5×10⁶ Da carried out chemical crosslinking reaction with different crosslinking agents at different crosslinking agent doses respectively under the freeze-drying condition to obtain the covalently cross-linked hyaluronic acid aerogels. The covalently cross-linked hyaluronic acid aerogels were soaked in PBS, so that the covalently cross-linked hyaluronic acid aerogels absorbed liquid and swelled to obtain the covalently cross-linked hyaluronic acid hydrogels. The degradation rates of the hydrogels were: 0.5% HA>1.0% HA>1.5% HA. It can be seen from FIG. 3-FIG. 8 that, the covalently cross-linked hyaluronic acid aerogels, obtained from HA with the molecular weight of 3.5×10⁵ Da and the concentration of 4% carried out chemical crosslinking reaction with the different crosslinking agents different crosslinking agent doses under the freeze-drying condition, soaked in PBS buffer solution to absorb liquid and swell to obtain the covalently cross-linked hyaluronic acid hydrogels, which had the longest the degradation times in the PBS buffer solution. While the covalently cross-linked hyaluronic acid aerogels, obtained from HA with the molecular weight of 1.5×10⁶ Da and the concentration of 1.5% carried out chemical crosslinking reaction with the different crosslinking agents at different crosslinking agent doses under the freeze-drying condition, soaked in PBS buffer solution to absorb liquid and swell to obtain the covalently cross-linked hyaluronic acid hydrogels, which had the longest the degradation times in the PBS buffer solution.

### Example 74: Experiment on Liquid-Absorbing Properties of Aerogels

The liquid-absorbing properties of the covalently cross-linked hyaluronic acid aerogels obtained by the freeze-drying reaction between hyaluronic acid with the molecular weight of 1.5×10⁶ Da and the concentration of 1.5% and different crosslinking agents at different crosslinking agent doses were tested, meanwhile the liquid-absorbing properties of the covalently cross-linked hyaluronic acid aerogels obtained by the freeze-drying reaction between hyaluronic acid with the molecular weight of 3.5×10⁵ Da and the concentration of 4.0% and different crosslinking agents at different crosslinking agent doses were tested.

1. The covalently cross-linked hyaluronic acid aerogels prepared in examples 9-12, 21-24, 33-36, 45-48, 57-60 and 69-72 were taken and cut into the size of 0.5 cm×0.5 cm respectively, and weights were measured and recorded as Wₒ (g) in a dry state under room temperature. The samples to be tested were put into culture dishes, and weights were measured and recorded as W_{d} (g).

2. An excessive amount of PBS buffer solution was added into each culture dish containing the covalently cross-linked hyaluronic acid aerogel, so that the covalently cross-linked hyaluronic acid aerogels absorbed liquid and swelled to obtain the covalently cross-linked hyaluronic acid hydrogels.

3. The PBS buffer solution in the culture dishes and attached to the surface of the hydrogels was completely sucked using filter paper and a disposable straw, weights were measured and recorded as Wₛ (g), and an excessive amount of PBS buffer solution was added into each culture dish again. Weights were measured once every 10 minutes, and an excessive amount of PBS buffer solution was added into each culture dish until the weight of the hydrogel was kept constant. The experiment was repeated three times.

4. The liquid-absorbing property of each aerogel was calculated using formula 1:

liquid-absorbing property=(Wₛ−W_{d})/W_{d} (formula 1)

wherein Wₛ is hydrogel weight; and W_{d} is aerogel weight. The experimental results are shown in Table 2 below.

TABLE 2
Liquid-absorbing Properties of Covalently cross-linked Aerogels

| | | Liquid-absorbing |
|---|---|---|
| Example | Sample Name | Property |
| 9 | HA³⁵-B-4-1 | 40.9 ± 5.33 |
| 10 | HA³⁵-B-4-2 | 35.3 ± 4.92 |
| 11 | HA³⁵-B-4-3 | 37.6 ± 1.32 |
| 12 | HA³⁵-B-4-4 | 29.2 ± 4.79 |
| 21 | HA³⁵-G-4-1 | 39.8 ± 0.71 |
| 22 | HA³⁵-G-4-2 | 37.3 ± 1.40 |
| 23 | HA³⁵-G-4-3 | 38.3 ± 1.78 |
| 24 | HA³⁵-G-4-4 | 29.3 ± 3.43 |
| 33 | HA³⁵-P500-4-1 | 40.6 ± 6.32 |
| 34 | HA³⁵-P500-4-2 | 35.9 ± 1.97 |
| 35 | HA³⁵-P500-4-3 | 36.3 ± 3.54 |
| 36 | HA³⁵-P500-4-4 | 33.0 ± 1.95 |
| 45 | HA¹⁵⁰-B-3-1 | 28.9 ± 1.81 |
| 46 | HA¹⁵⁰-B-3-2 | 35.7 ± 7.69 |
| 47 | HA¹⁵⁰-B-3-3 | 36.5 ± 6.44 |
| 48 | HA¹⁵⁰-B-3-4 | 23.1 ± 4.66 |
| 57 | HA¹⁵⁰-G-3-1 | 44.7 ± 6.13 |
| 58 | HA¹⁵⁰-G-3-2 | 37.6 ± 1.40 |
| 59 | HA¹⁵⁰-G-3-3 | 33.3 ± 2.13 |
| 60 | HA¹⁵⁰-G-3-4 | 23.1 ± 1.10 |
| 69 | HA¹⁵⁰-P500-3-1 | 30.2 ± 2.10 |
| 70 | HA¹⁵⁰-P500-3-2 | 34.5 ± 0.35 |
| 71 | HA¹⁵⁰-P500-3-3 | 36.4 ± 1.64 |
| 72 | HA¹⁵⁰-P500-3-4 | 22.4 ± 0.87 |

Liquid-absorbing property: the weight of liquid absorbed by every 1 g of covalently cross-linked hyaluronic acid aerogel (g)

The experimental results indicate that, all the covalently cross-linked hyaluronic acid aerogels absorbed liquid and swelled completely to form the covalently cross-linked hyaluronic acid hydrogels within 10 minutes. It can be seen from the data in Table 2 that, the weights of liquid absorbed by the covalently cross-linked hyaluronic acid aerogels are 22 to 45 times the weights of the aerogels, indicating that the covalently cross-linked hyaluronic acid aerogels have good liquid-absorbing capability.

### Example 75: Rheological Experiment on Hyaluronic Acid Hydrogels

Rheological study is an effective tool to analyze the structure and properties of a viscoelastic material. During a study on a hydrogel, the formation time, frequency and strain of the hydrogel were swept to obtain its storage modulus (G′) and loss modulus (G″) change curve to represent the properties of the hydrogel.

The parameters in the rheological test were set as: sweep mode: oscillation; rotor: PP25, gap 1 mm; point setting frequency: 1/20 s; test temperature: 20° C.

Time sweep: Frequency (f) was 1 Hz, strain was 1%, and points were set at a set time;

Frequency sweep: Frequency (f) was 10 Hz to 0.01 Hz, strain was 1%, and 19 points were set in total, no time setting;

Strain sweep: Frequency (f) was 1 Hz, strain was 0.1% to 100%, and 19 points were set in total, no time setting. The experimental samples were grouped as Table 3.

TABLE 3
Parameters of Experimental Samples

| Example | Sample Name | Condition |
|---|---|---|
| 9 | HA³⁵-B-4-1 | a |
| \ | 4% HA³⁵+ 0.1% BDDE | b |
| 10 | HA³⁵-B-4-2 | a |
| \ | 4% HA³⁵ + 0.2% BDDE | b |
| 11 | HA³⁵-B-4-3 | a |
| \ | 4% HA³⁵ + 0.4% BDDE | b |
| 12 | HA³⁵-B-4-4 | a |
| \ | 4% HA³⁵ + 1.0% BDDE | b |
| 21 | HA³⁵-G-4-1 | a |
| \ | 4% HA³⁵+ 0.1% GDGE | b |
| 22 | HA³⁵-G-4-2 | a |
| \ | 4% HA³⁵+ 0.2% GDGE | b |
| 23 | HA³⁵-G-4-3 | a |
| \ | 4% HA³⁵+ 0.4% GDGE | b |
| 24 | HA³⁵-G-4-4 | a |
| \ | 4% HA³⁵ + 1.0% GDGE | b |
| 33 | HA³⁵-P500-4-1 | a |
| \ | 4% HA³⁵ + 0.1% P500 | b |
| 34 | HA³⁵-P500-4-2 | a |
| \ | 4% HA³⁵ + 0.2% P500 | b |
| 35 | HA³⁵-P500-4-3 | a |
| \ | 4% HA³⁵ + 0.4% P500 | b |
| 36 | HA³⁵-P500-4-4 | a |
| \ | 4% HA³⁵ + 1.0% P500 | b |
| 45 | HA¹⁵⁰-B-3-1 | a |
| \ | 1.5% HA¹⁵⁰ + 0.1% BDDE | b |
| 46 | HA¹⁵⁰-B-3-2 | a |
| \ | 1.5% HA¹⁵⁰ + 0.2% BDDE | b |
| 47 | HA¹⁵⁰-B-3-3 | a |
| \ | 1.5% HA¹⁵⁰ + 0.4% BDDE | b |
| 48 | HA¹⁵⁰-B-3-4 | a |
| \ | 1.5% HA¹⁵⁰ + 1.0% BDDE | b |
| 57 | HA¹⁵⁰-G-3-1 | a |
| \ | 1.5% HA¹⁵⁰ + 0.1% GDGE | b |
| 58 | HA¹⁵⁰-G-3-2 | a |
| \ | 1.5% HA¹⁵⁰ + 0.2% GDGE | b |
| 59 | HA¹⁵⁰-G-3-3 | a |
| \ | 1.5% HA¹⁵⁰ + 0.4% GDGE | b |
| 60 | HA¹⁵⁰-G-3-4 | a |
| \ | 1.5% HA¹⁵⁰ + 1.0% GDGE | b |
| 69 | HA¹⁵⁰-P500-3-1 | a |
| \ | 1.5% HA¹⁵⁰ + 0.1% P500 | b |
| 70 | HA¹⁵⁰-P500-3-2 | a |
| \ | 1.5% HA¹⁵⁰ + 0.2% P500 | b |
| 71 | HA¹⁵⁰-P500-3-3 | a |
| \ | 1.5% HA¹⁵⁰ + 0.4% P500 | b |
| 72 | HA¹⁵⁰-P500-3-4 | a |
| \ | 1.5% HA¹⁵⁰ + 1.0%P500 | b |

a. adding water after freeze-drying; b. standing under room temperature for 72 hours.

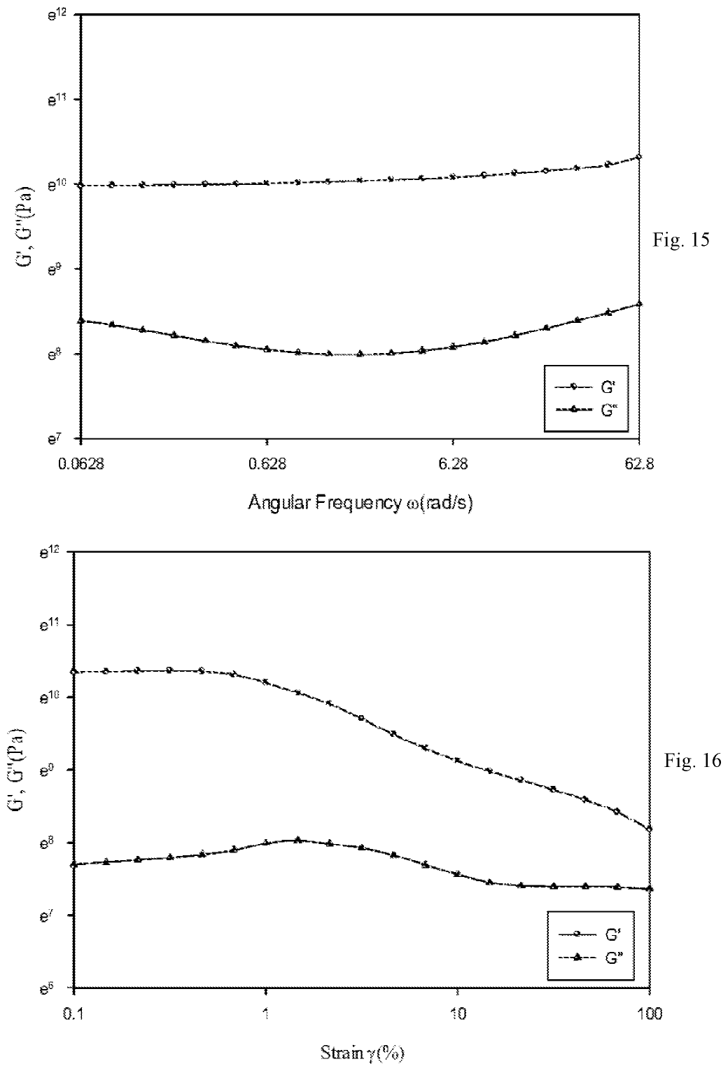

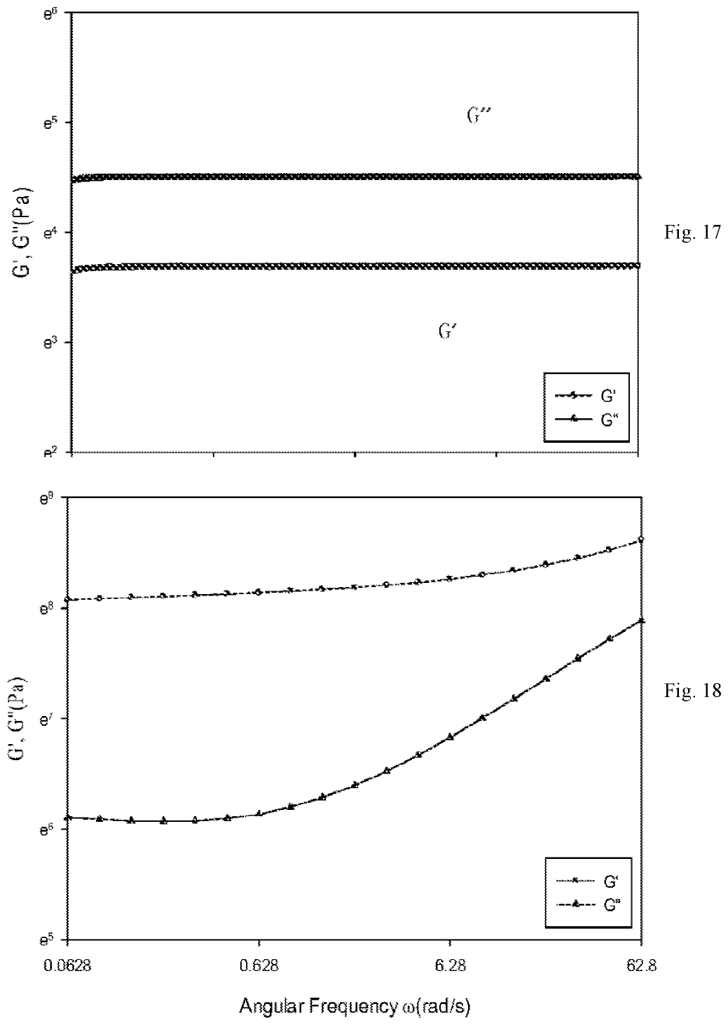

It can be known from time sweep in FIG. 15 that G′ of the “4% HA³⁵+0.1% BDDE” sample is less than G″, indicating that this sample is not a hydrogel. It can be discovered in frequency sweep in FIG. 16 that G′ is greater than G″, which can prove that the hyaluronic acid hydrogel prepared in example 9 is a chemically cross-linked hydrogel. FIG. 15 and FIG. 16 indicate that the present invention promotes the chemical crosslinking between the crosslinking agents and hyaluronic acid through the freeze-drying technique. Strain sweep in FIG. 17 indicates that under the condition of fixed frequency and temperature, as the shearing force is gradually increased, the storage modulus of the covalently cross-linked hyaluronic acid hydrogel prepared in example 9 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is average.

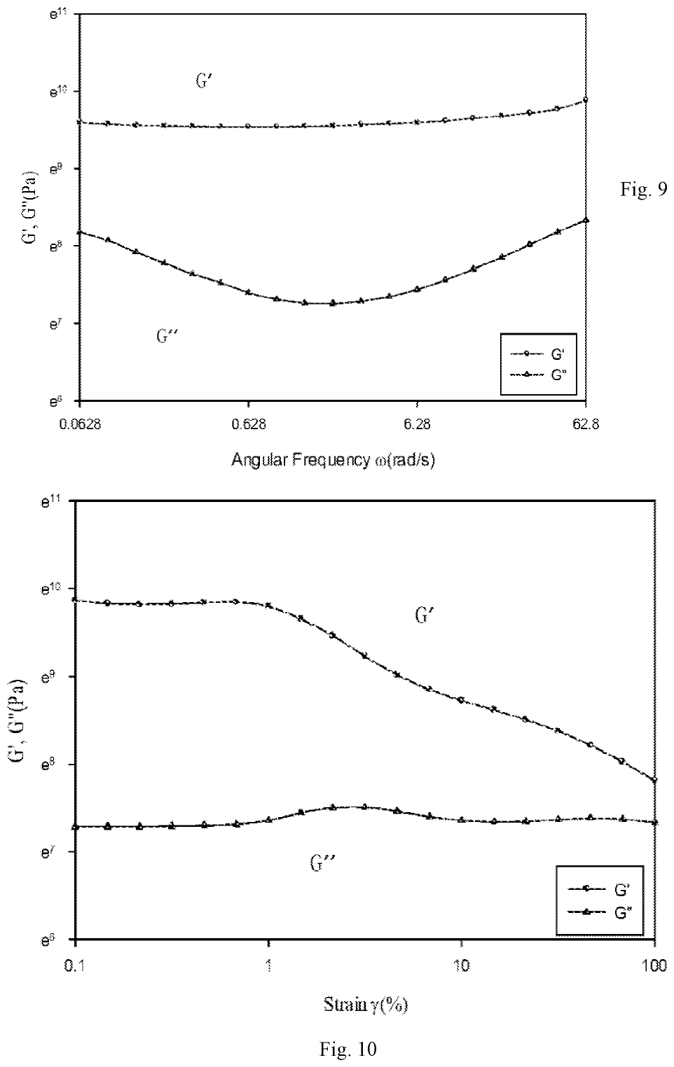

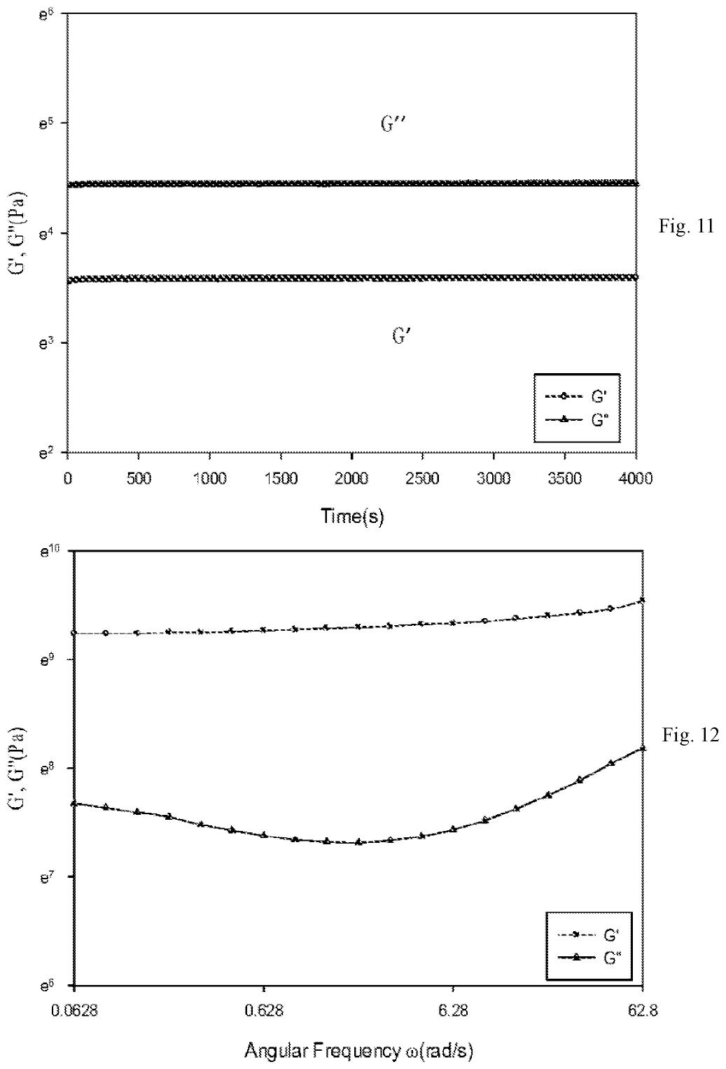

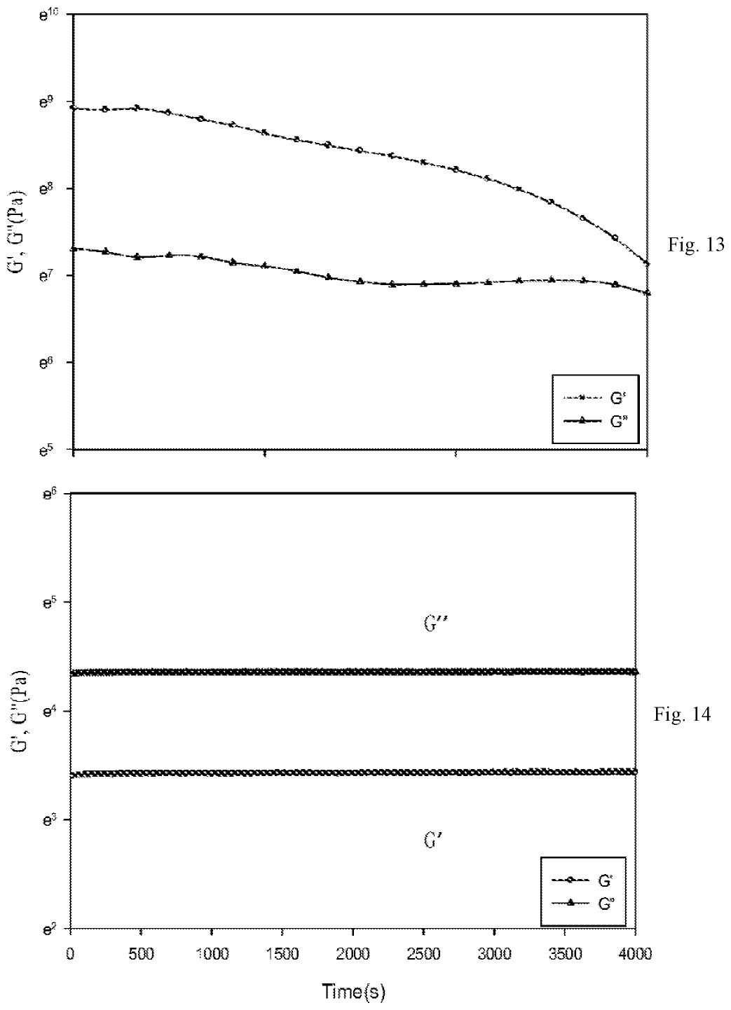

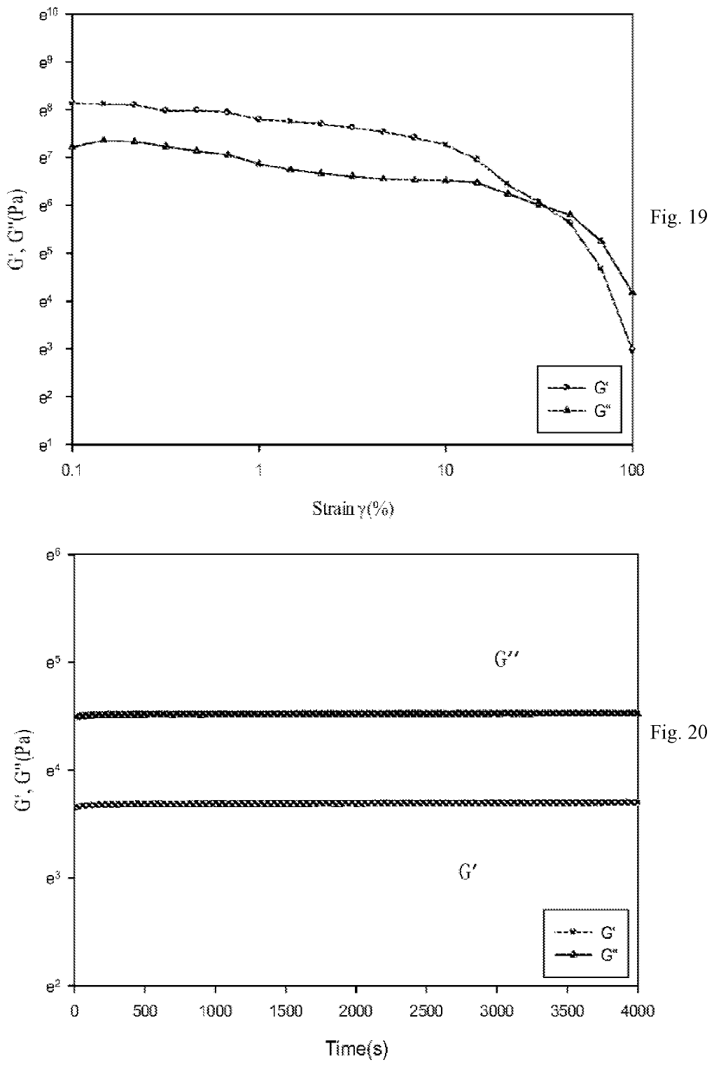

It can be known from time sweep in FIG. 8, FIG. 11 and FIG. 14 that G′ of the “4.0% HA³⁵+0.2% BDDE”, “4.0% HA³⁵+0.4% BDDE” and “4.0% HA³⁵+1.0% BDDE” samples are less than G″, indicating that these samples are not hydrogels. It can be discovered in frequency sweep in FIG. 9, FIG. 12 and FIG. 15 that G′ is greater than G″, which can prove that the hyaluronic acid hydrogels prepared in examples 10, 11 and 12 are chemically cross-linked hydrogels. FIG. 18 and FIG. 19 indicate that the present invention promotes the chemical crosslinking between the crosslinking agents and hyaluronic acid through the freeze-drying technique. FIG. 11 and FIG. 12 indicate that the present invention promotes the chemical crosslinking between the crosslinking agents and hyaluronic acid through the freeze-drying technique. FIG. 14 and FIG. 15 indicate that the present invention promotes the chemical crosslinking between the crosslinking agents and hyaluronic acid through the freeze-drying technique. Strain sweep in FIG. 10 indicates that the covalently cross-linked hyaluronic acid hydrogel prepared in example 10 has excellent viscoelasticity. Strain sweep in FIG. 13 indicates that the covalently cross-linked hyaluronic acid hydrogel prepared in example 11 has excellent viscoelasticity. Strain sweep in FIG. 16 indicates that the covalently cross-linked hyaluronic acid hydrogel prepared in example 12 has excellent viscoelasticity.

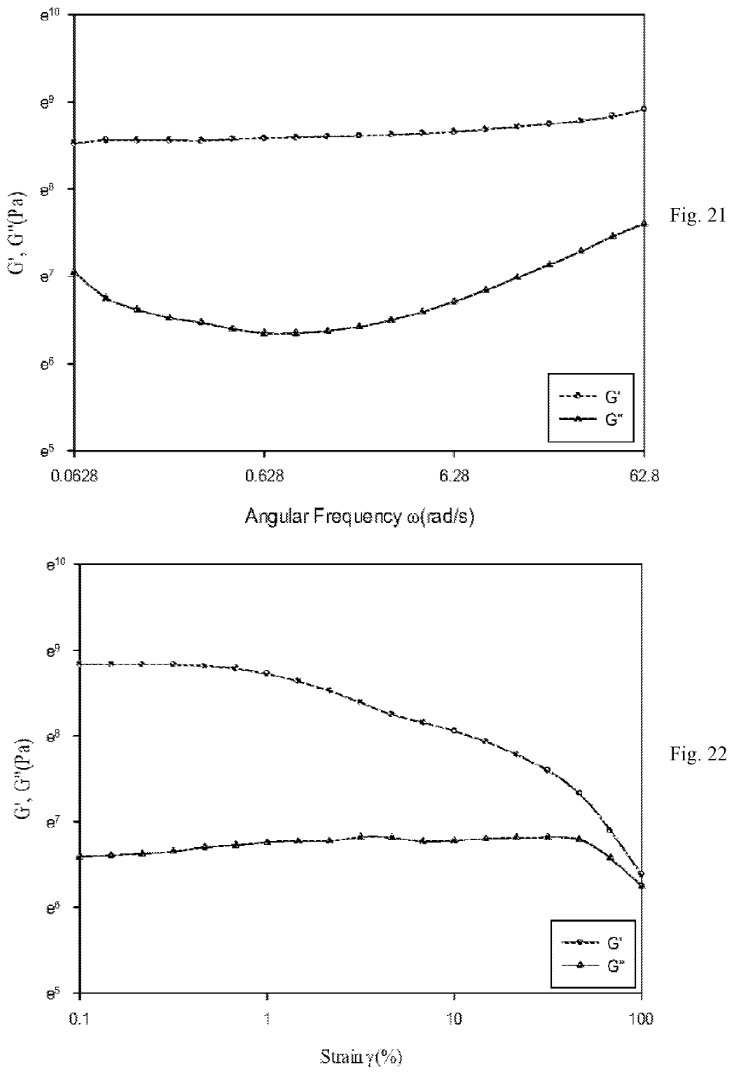

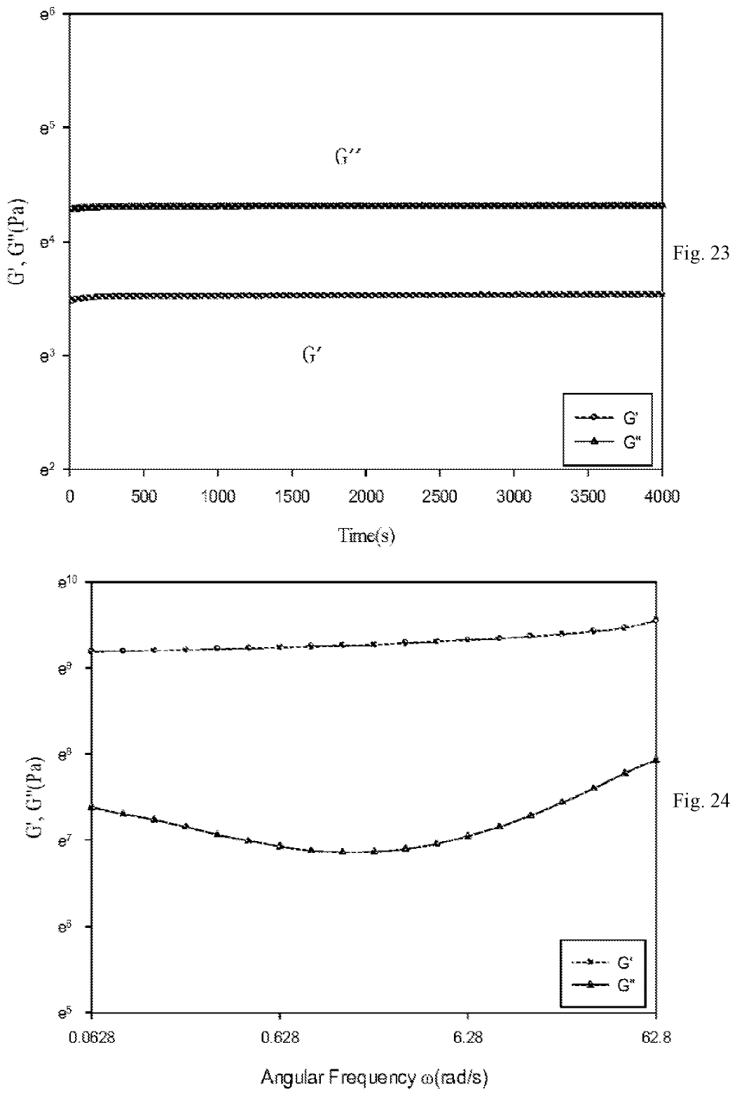

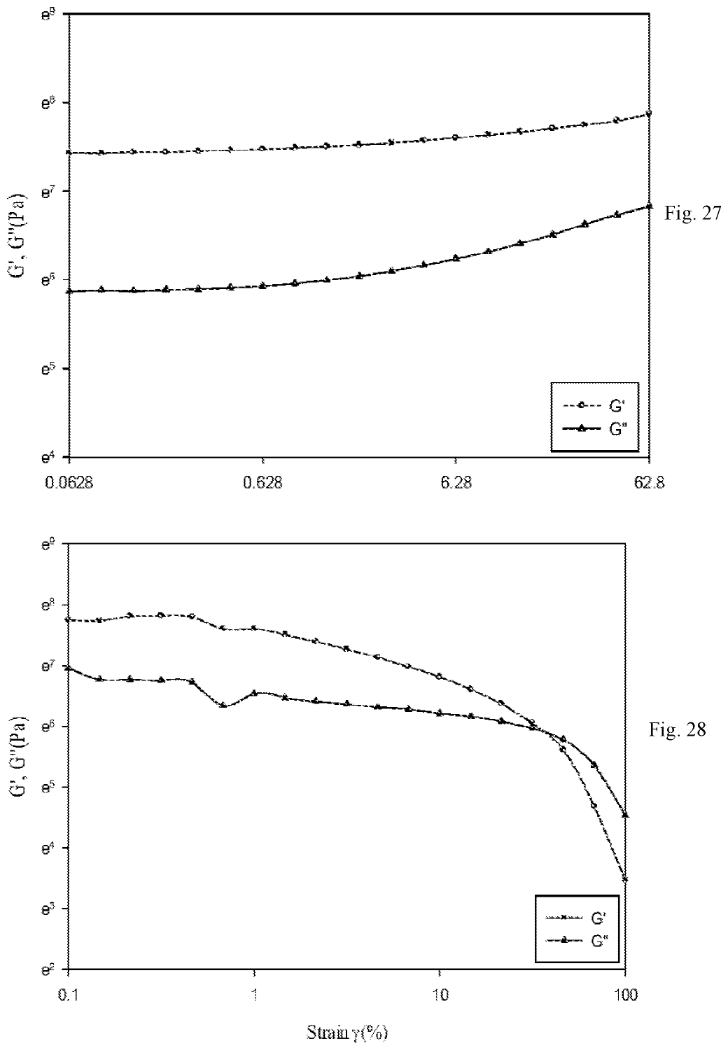

It can be known from time sweep in FIG. 17, FIG. 20, FIG. 23 and FIG. 26 that G′ of the “4.0% HA³⁵+0.1% GDGE”, “4.0% HA³⁵+0.2% GDGE”, “4.0% HA³⁵+0.4% GDGE” and “4.0% HA³⁵+1.0% GDGE” samples are less than G″, indicating that these samples are not hydrogels. It can be discovered in frequency sweep in FIG. 18, FIG. 21, FIG. 24 and FIG. 27 that G′ are greater than G″, which can prove that the hyaluronic acid hydrogels prepared in examples 21, 22, 23 and 24 are chemically cross-linked hydrogels. FIG. 17 and FIG. 18, FIG. 20 and FIG. 21, FIG. 23 and FIG. 24 and FIG. 26 and FIG. 27 indicate that the present invention promotes the chemical crosslinking between the crosslinking agents and hyaluronic acid through the freeze-drying technique.

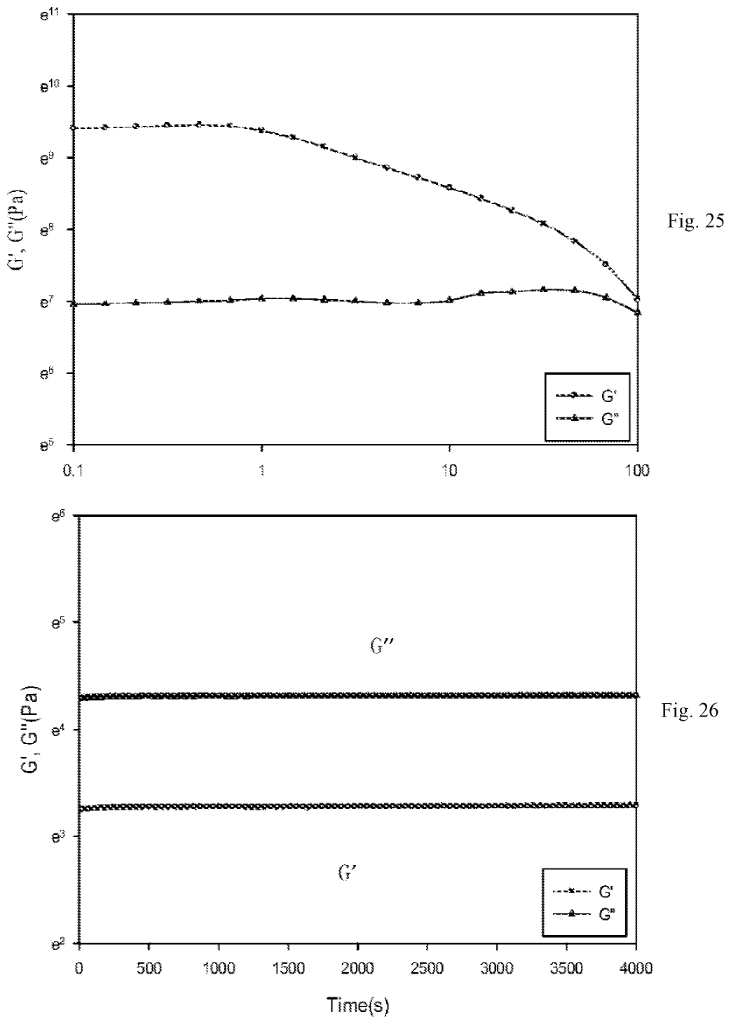

Strain sweep in FIG. 19 indicates that under the condition of fixed frequency and temperature, as shearing force is gradually increased, the storage modulus of the covalently cross-linked hyaluronic acid hydrogel prepared in example 21 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is average. Strain sweep in FIG. 22 indicates that the covalently cross-linked hyaluronic acid hydrogel prepared in example 22 has excellent viscoelasticity. Strain sweep in FIG. 25 indicates that the covalently cross-linked hyaluronic acid hydrogel prepared in example 23 has excellent viscoelasticity. Strain sweep in FIG. 38 indicates that under the condition of fixed frequency and temperature, as shearing force is gradually increased, the storage modulus of the covalently cross-linked hyaluronic acid hydrogel prepared in example 24 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is average.

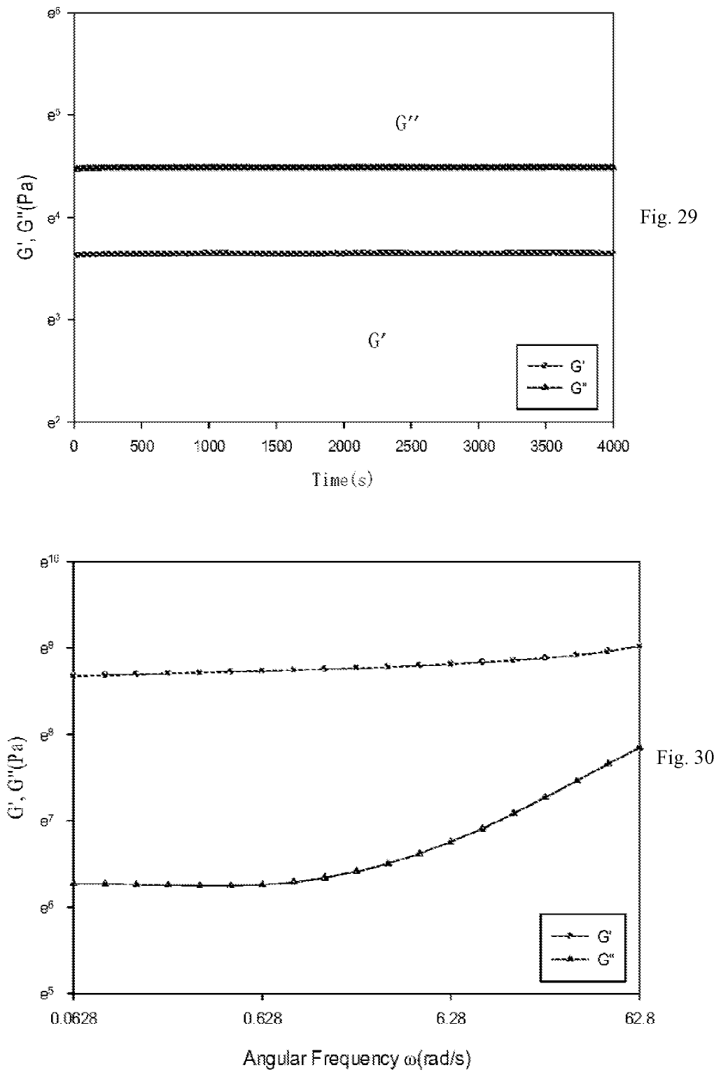

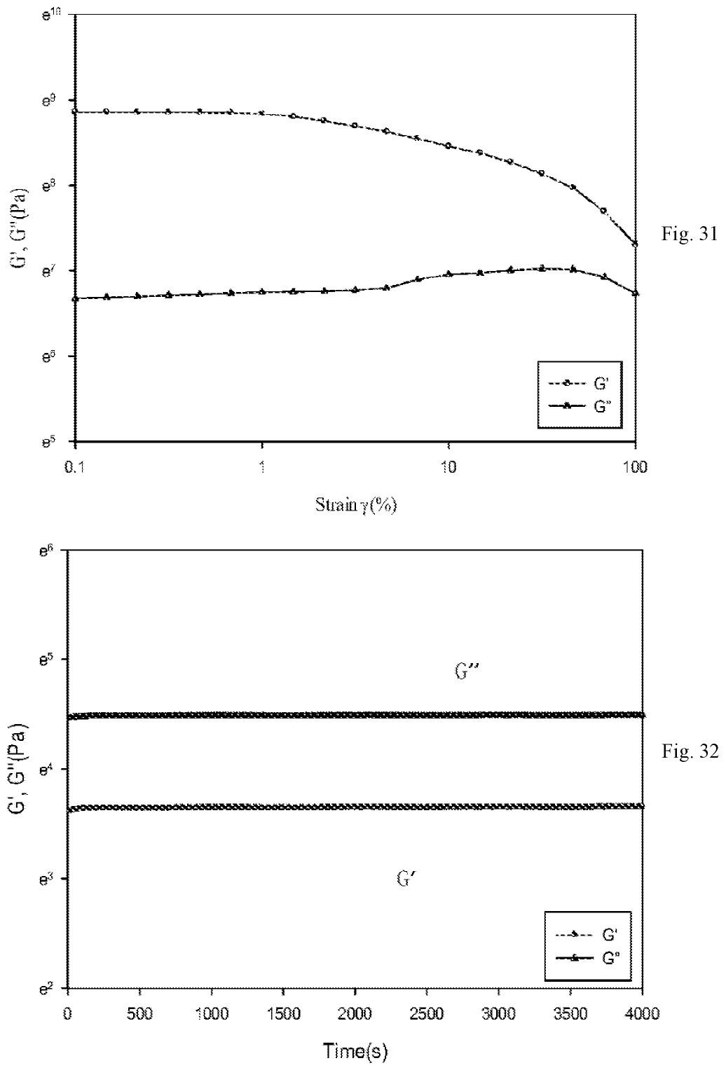

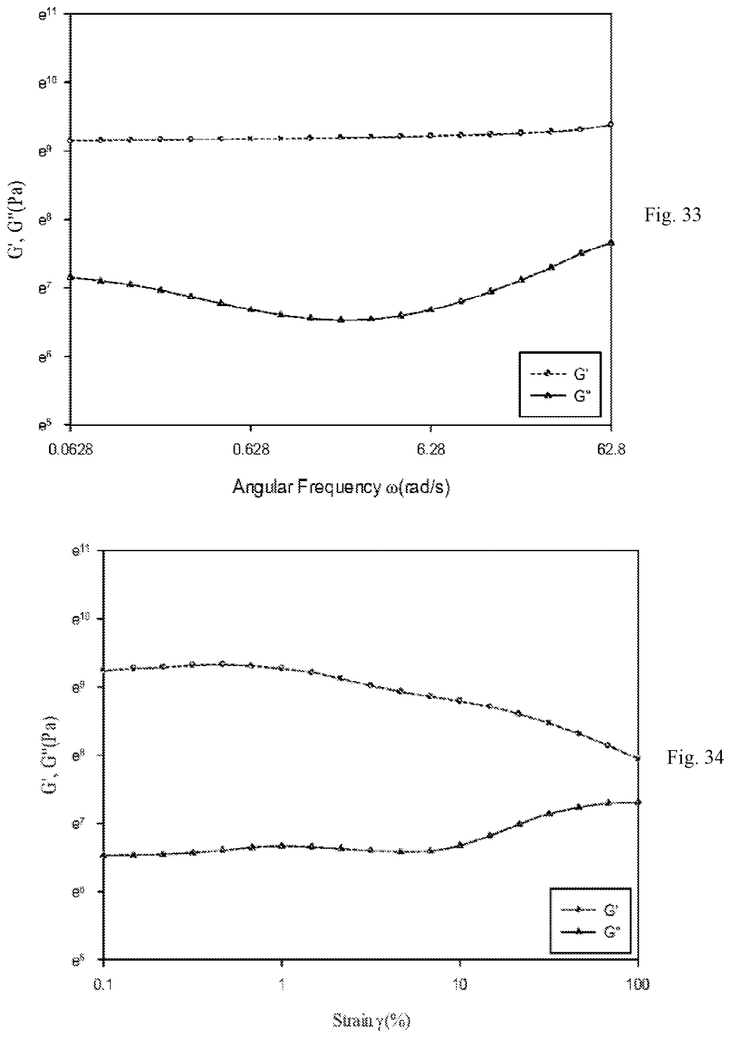

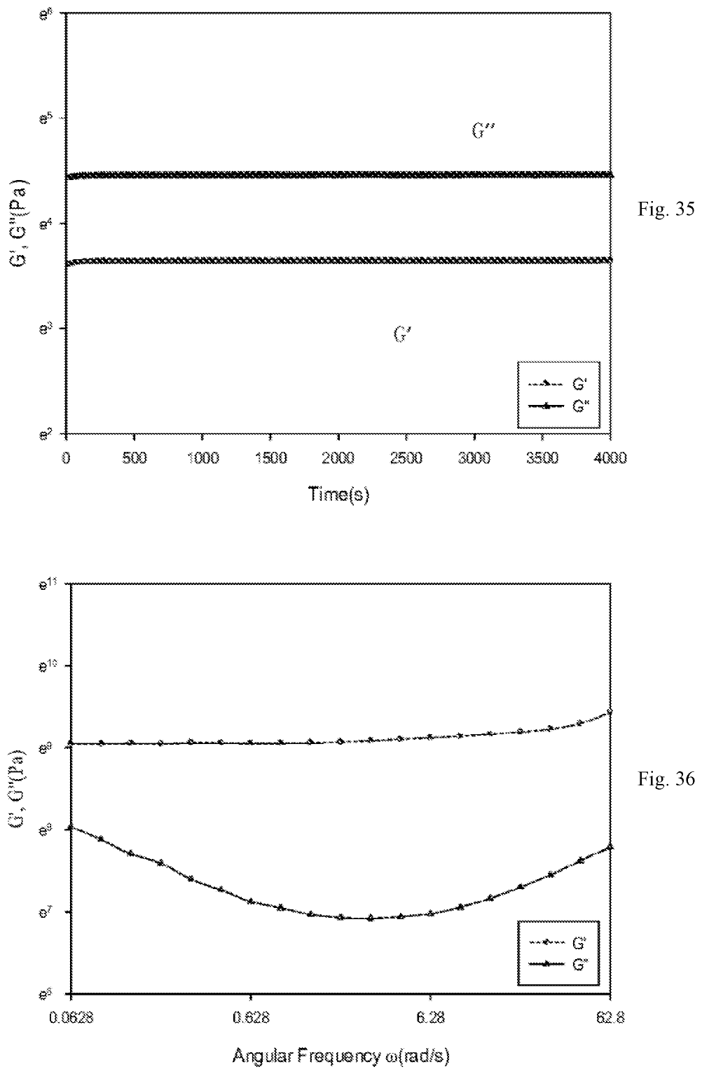

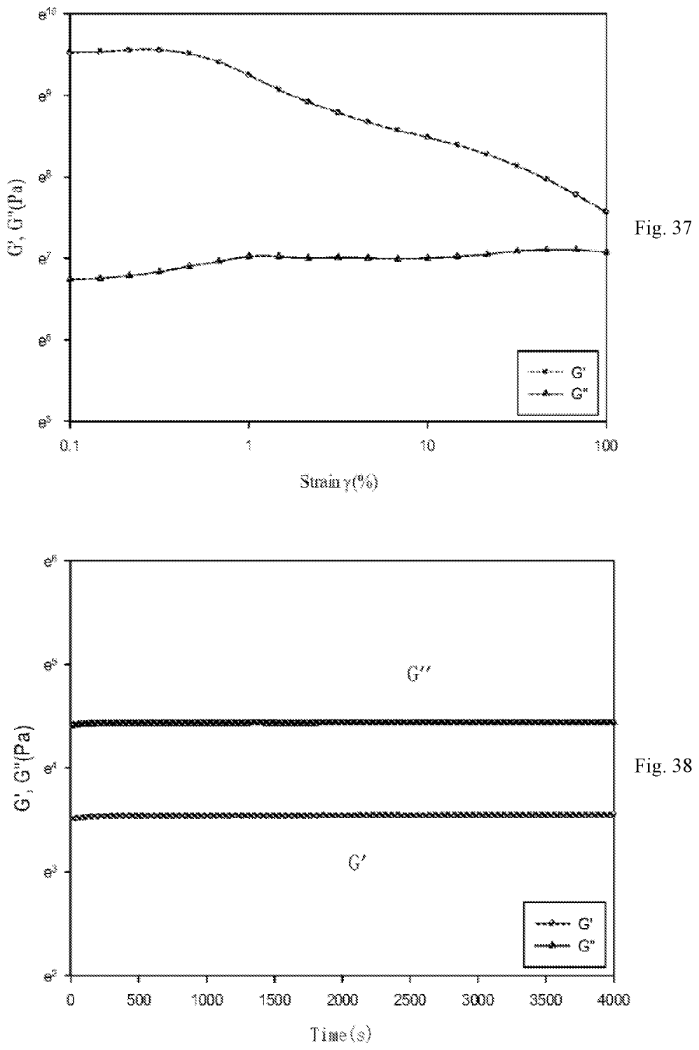

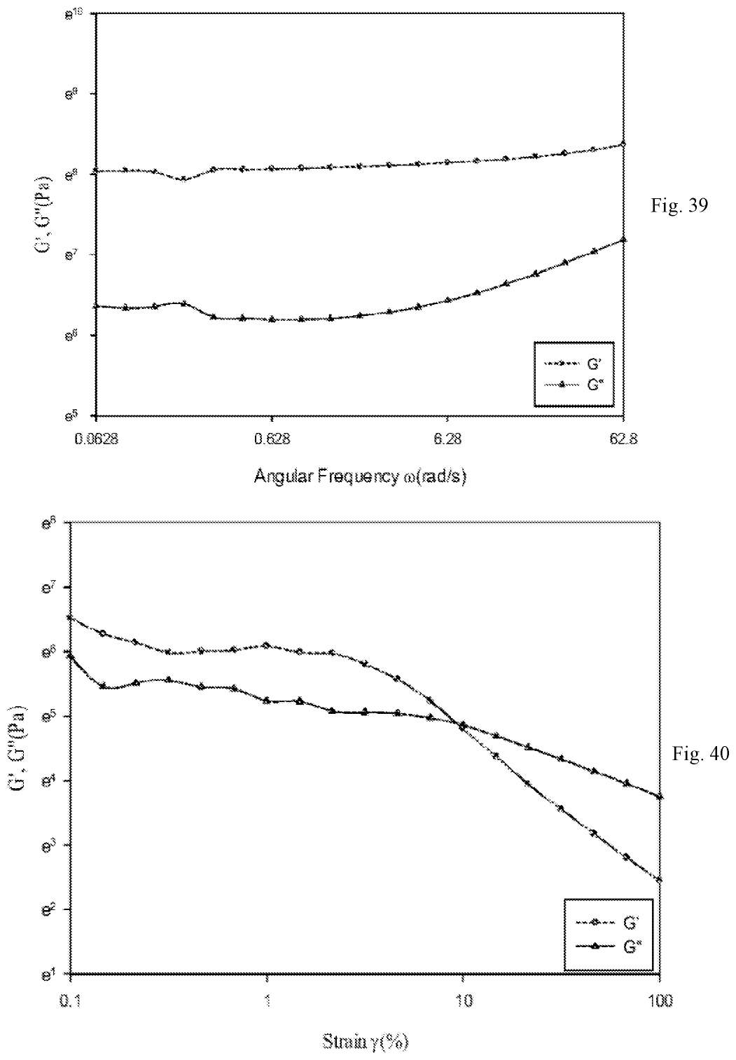

It can be known from time sweep in FIG. 29, FIG. 32, FIG. 35 and FIG. 38 that G′ of the “4.0% HA³⁵+0.1% PEG500”, “4.0% HA³⁵+0.2% PEG500”, “4.0% HA³⁵+0.4% PEG500” and “4.0% HA³⁵+1.0% PEG500” samples are less than G″, indicating that these samples are not hydrogels. It can be discovered in frequency sweep in FIG. 30, FIG. 33, FIG. 36 and FIG. 39 that G′ are greater than G″, which can prove that the hyaluronic acid hydrogels prepared in examples 33, 34, 35 and 36 are chemically cross-linked hydrogels. FIG. 29 and FIG. 30, FIG. 32 and FIG. 33, FIG. 35 and FIG. 36 and FIG. 38 and FIG. 39 respectively indicate that the present invention promotes the chemical crosslinking between the crosslinking agents and hyaluronic acid through the freeze-drying technique. Strain sweep in FIG. 31 indicates that the covalently cross-linked hyaluronic acid hydrogel prepared in example 33 has excellent viscoelasticity. Strain sweep in FIG. 34 indicates that the covalently cross-linked hyaluronic acid hydrogel prepared in example 34 has excellent viscoelasticity. Strain sweep in FIG. 37 indicates that the covalently cross-linked hyaluronic acid hydrogel prepared in example 35 has excellent viscoelasticity. Strain sweep in FIG. 40 indicates that under the condition of fixed frequency and temperature, as shearing force is gradually increased, the storage modulus of the covalently cross-linked hyaluronic acid hydrogel prepared in example 36 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is average.

A comparative experiment of (a) water addition after freeze-drying and (b) 72 hours of standing under room temperature and a rheological study were also performed according to the formulas of hyaluronic acid (1.5×10⁶ Da, concentration: 1.5%) and the different crosslinking agents at different crosslinking agent doses and the reaction system used in Table 2, and the result also proves that freeze-drying promotes the formation of covalently cross-linked products, while 72 hours of standing under room temperature cannot form covalently cross-linked products.

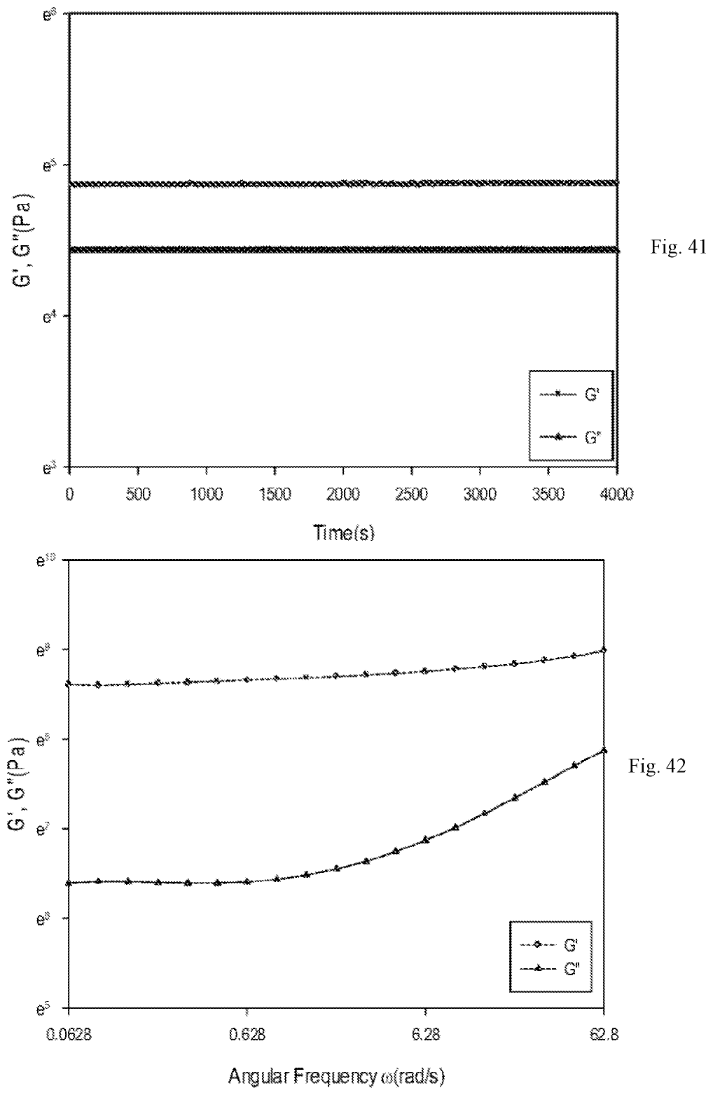

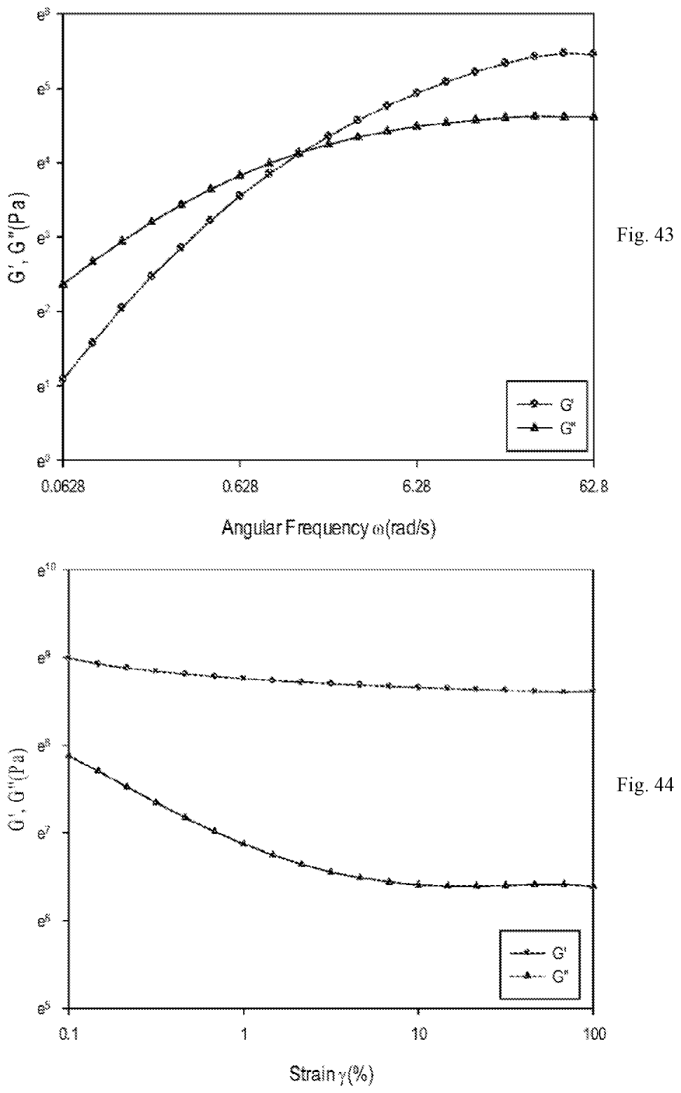

It can be known by observing time sweep in FIG. 41 that G′ of the “1.5% HA¹⁵⁰+0.1% BDDE” sample is greater than G″, indicating that this sample is a hydrogel. It can be discovered through frequency sweep in FIG. 42 that no matter how frequency changes, G′ is always greater than G″, indicating that the covalently cross-linked hyaluronic acid hydrogel prepared in example 45 is a chemically cross-linked hydrogel. It can be known through frequency sweep in FIG. 43 that with a decrease in frequency, the “1.5% HA¹⁵⁰+0.1% BDDE” sample is changed from the state of “G′ is greater than G“ ” into the state of “G′ is less than G”, indicating that the “1.5% HA¹⁵⁰+0.1% BDDE” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Meanwhile, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. It can be seen through strain sweep in FIG. 44 that when frequency and temperature do not change, although shearing force is gradually increased, the storage modulus (G′) of the hydrogel is still basically stable, which indicates that the network structure of the hydrogel will not change due to an increase in strain, as the network structure of this hydrogel has an excellent viscoelasticity.

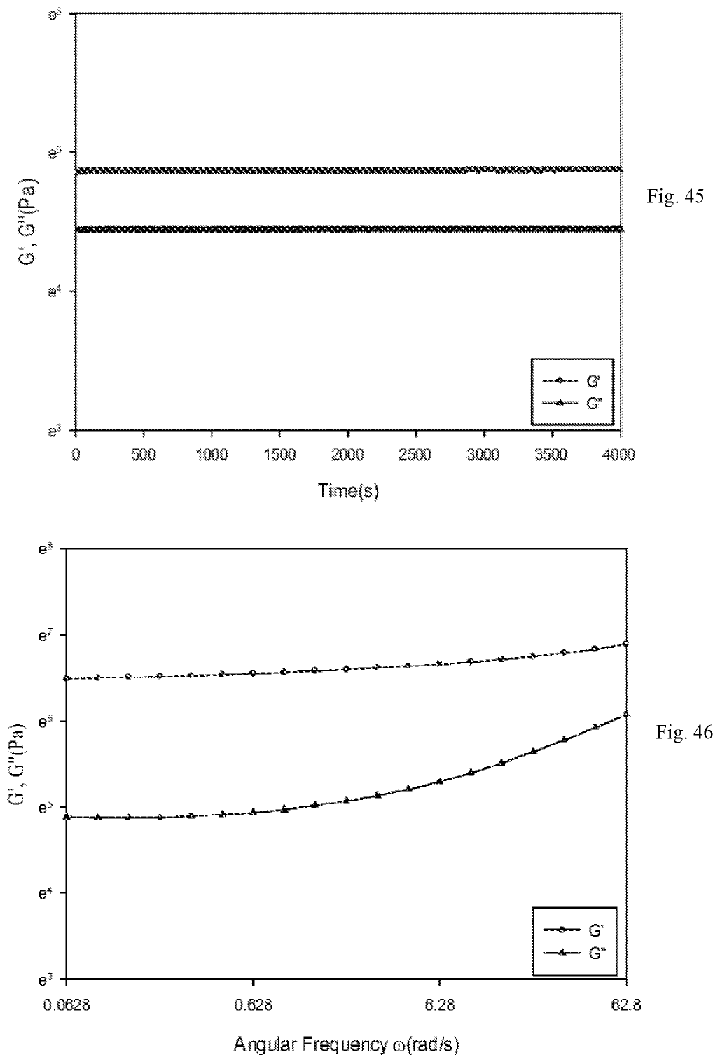

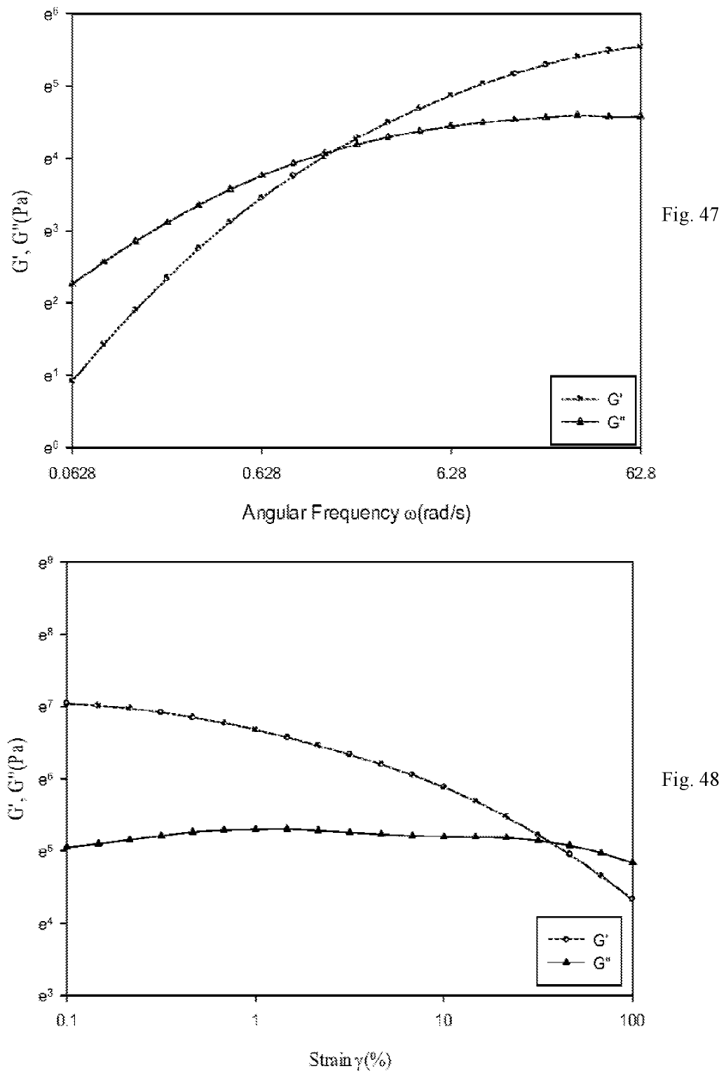

It can be known by observing time sweep in FIG. 45 that G′ of the “1.5% HA¹⁵⁰+0.2% BDDE” sample is greater than G″, indicating that this sample is a hydrogel. It can be discovered through frequency sweep in FIG. 46 that no matter how frequency changes, G′ is always greater than G″, indicating that the covalently cross-linked hyaluronic acid hydrogel prepared in example 46 is a chemically cross-linked hydrogel. It can be known through frequency sweep in FIG. 47 that with a decrease in frequency, the “1.5% HA¹⁵⁰+0.2% BDDE” sample is changed from the state of “G′ is greater than G“ ” into the state of “G′ is less than G”, indicating that the “1.5% HA¹⁵⁰+0.2% BDDE” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Meanwhile, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. It can be seen from strain sweep in FIG. 48 that under the condition of fixed frequency and temperature, as shearing force is gradually increased, the storage modulus of the covalently cross-linked hyaluronic acid hydrogel prepared in example 46 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is ordinary.

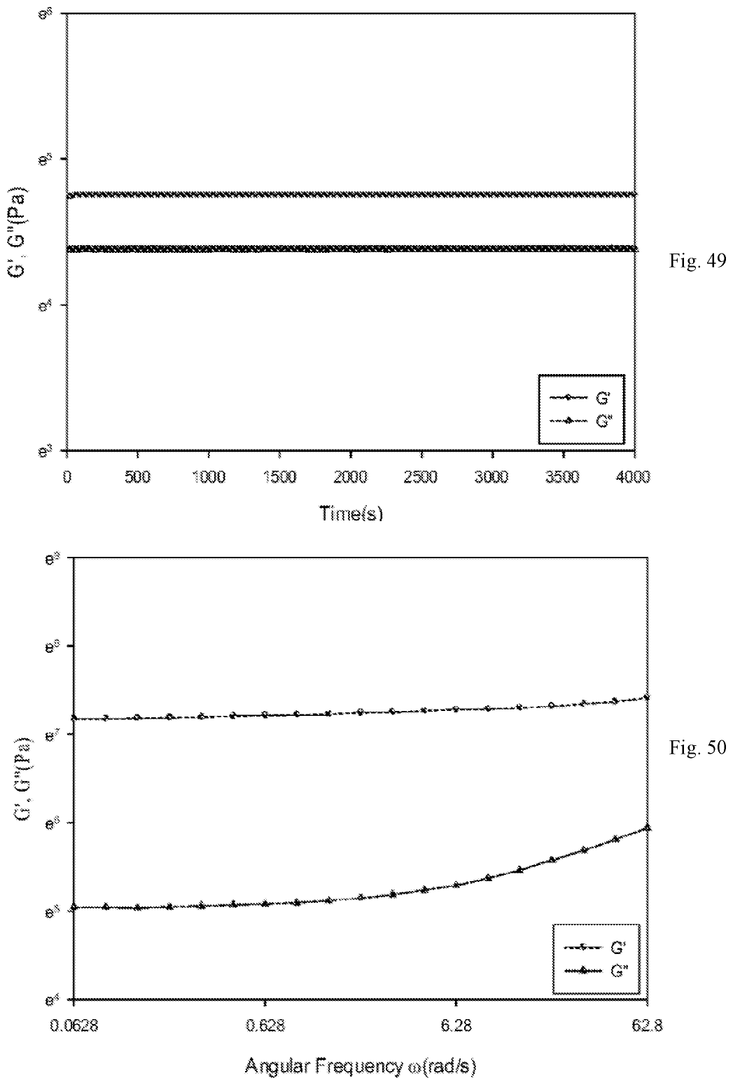

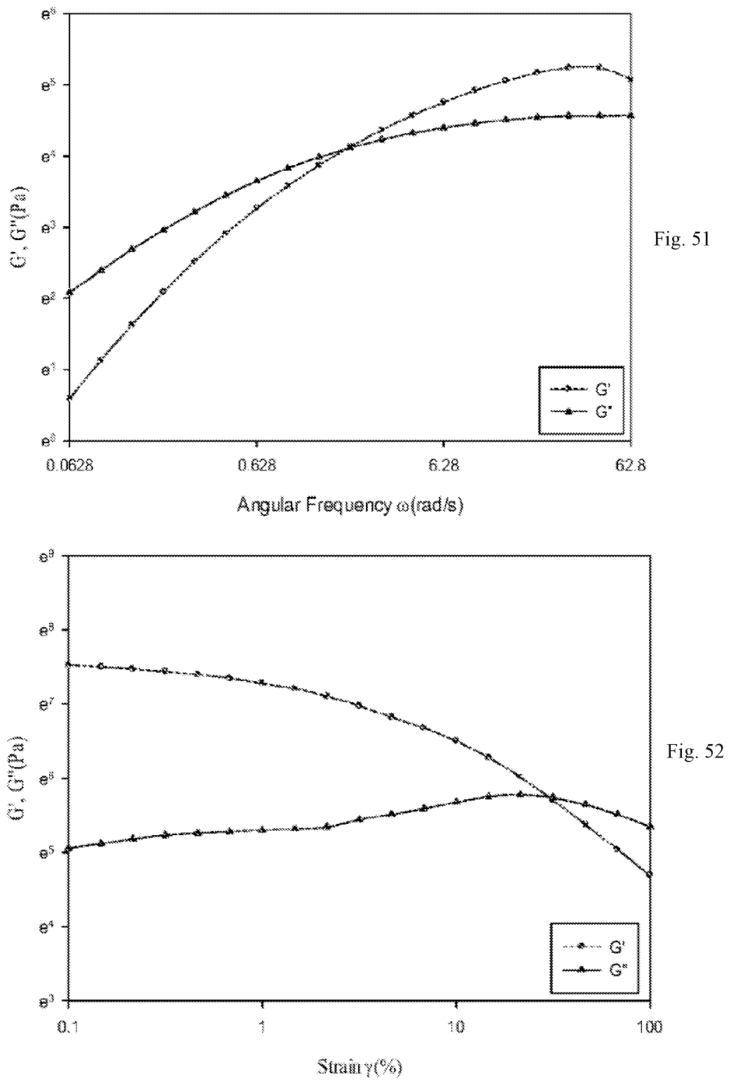

It can be known by observing time sweep in FIG. 49 that G′ of the “1.5% HA¹⁵⁰+0.4% BDDE” sample is greater than G″, indicating that this sample is a hydrogel. It can be discovered through frequency sweep in FIG. 50 that no matter how frequency changes, G′ is always greater than G″, indicating that the covalently cross-linked hyaluronic acid hydrogel prepared in example 47 is a chemically cross-linked hydrogel. It can be known through frequency sweep in FIG. 51 that with a decrease in frequency, the “1.5% HA¹⁵⁰+0.4% BDDE” sample is changed from the state of “G′ is greater than G“ ” into the state of “G′ is less than G”, indicating that the “1.5% HA¹⁵⁰+0.4% BDDE” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Meanwhile, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. It can be seen from strain sweep in FIG. 52 that under the condition of fixed frequency and temperature, as shearing force is gradually increased, the storage modulus of the covalently cross-linked hyaluronic acid hydrogel prepared in example 47 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is ordinary.

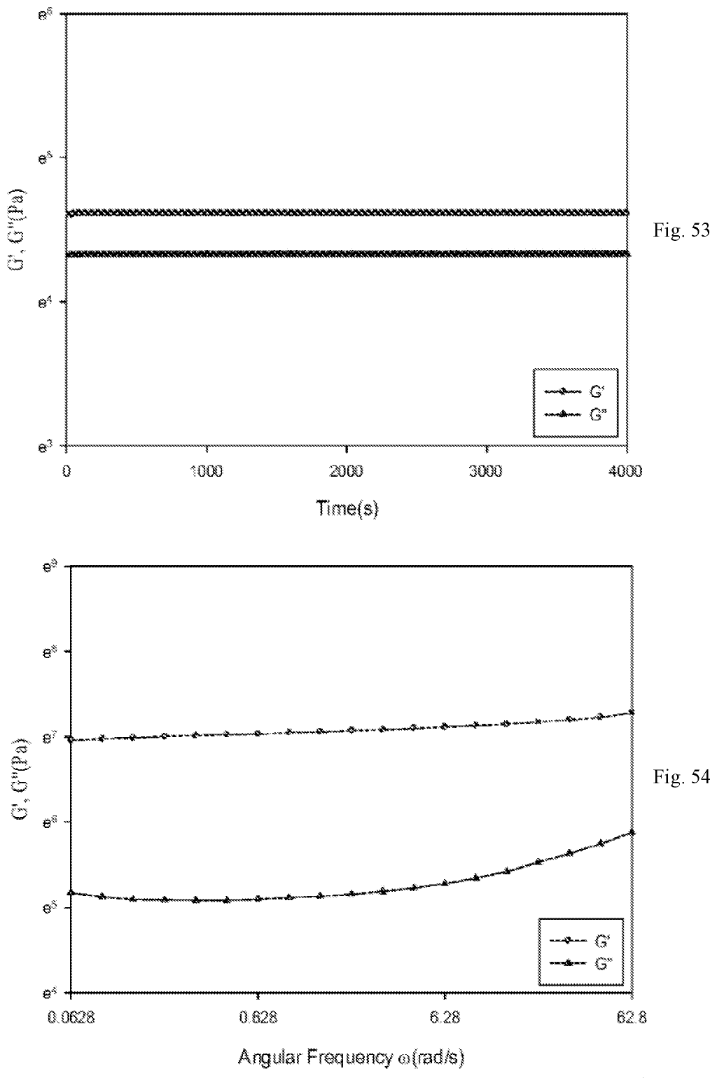

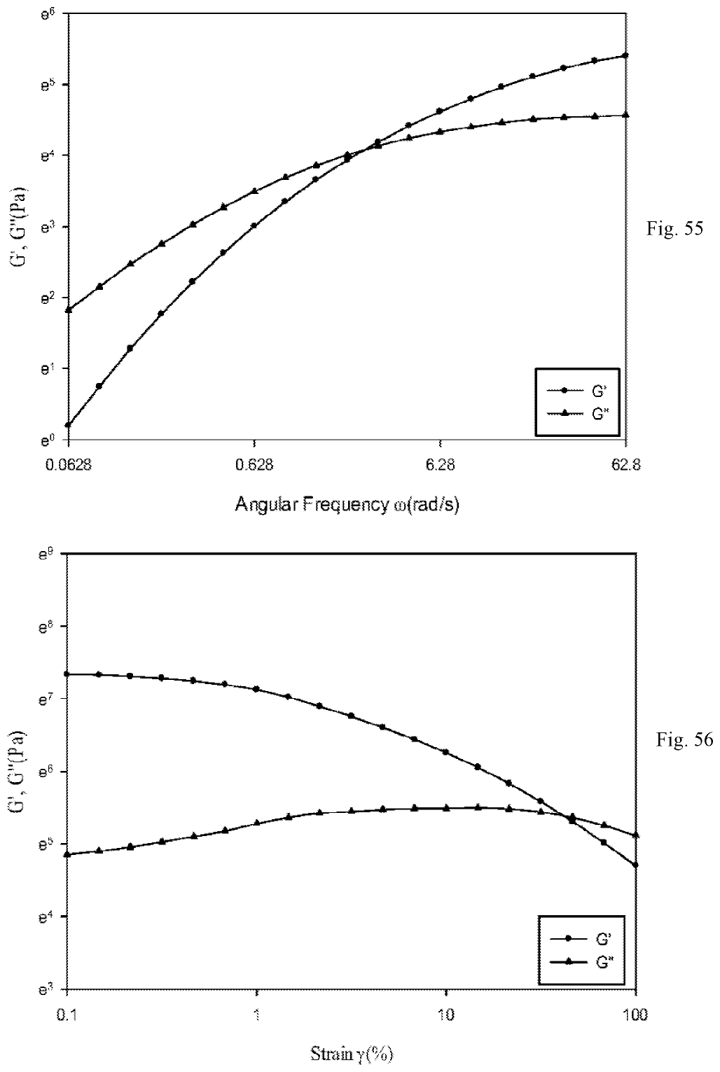

It can be known by observing time sweep in FIG. 53 that G′ of the “1.5% HA¹⁵⁰+1.0% BDDE” sample is greater than G″, indicating that this sample is a hydrogel. It can be discovered through frequency sweep in FIG. 54 that no matter how frequency changes, G′ is always greater than G″, indicating that the covalently cross-linked hyaluronic acid hydrogel prepared in example 48 is a chemically cross-linked hydrogel. It can be known through frequency sweep in FIG. 55 that with a decrease in frequency, the “1.5% HA¹⁵⁰+1.0% BDDE” sample is changed from the state of “G′ is greater than G“ ” into the state of “G′ is less than G”, indicating that the “1.5% HA¹⁵⁰+1.0% BDDE” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Meanwhile, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. Strain sweep in FIG. 56 indicates that under the condition of fixed frequency and temperature, as shearing force is gradually increased, the storage modulus of the covalently cross-linked hyaluronic acid hydrogel prepared in example 48 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is ordinary.

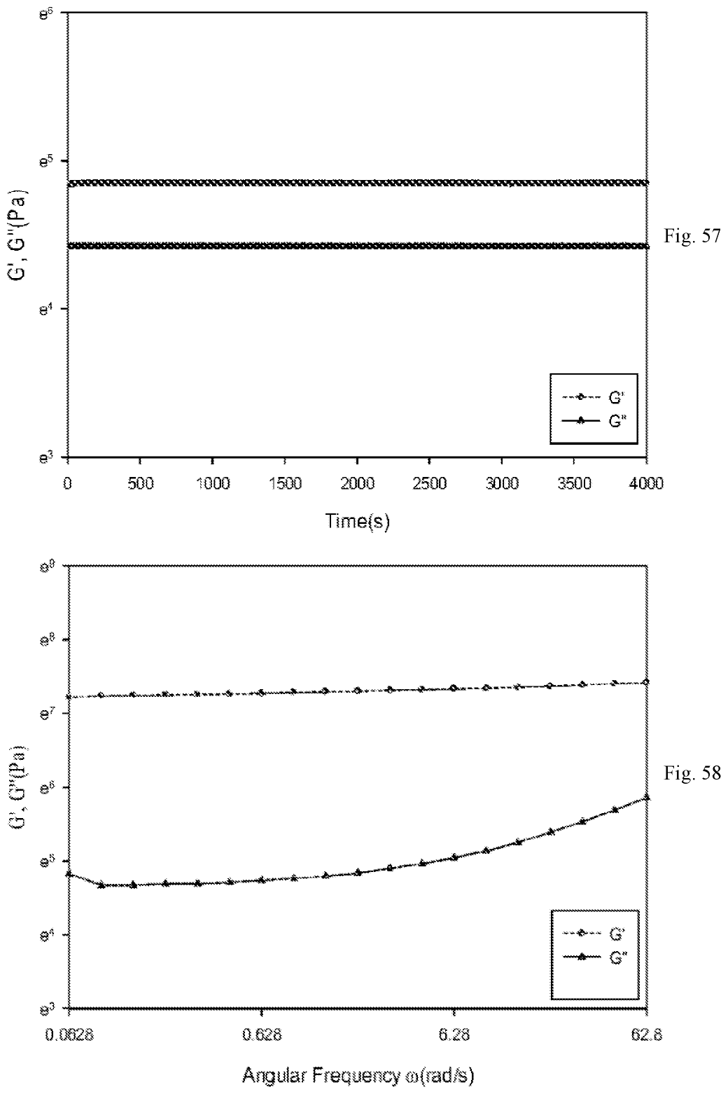

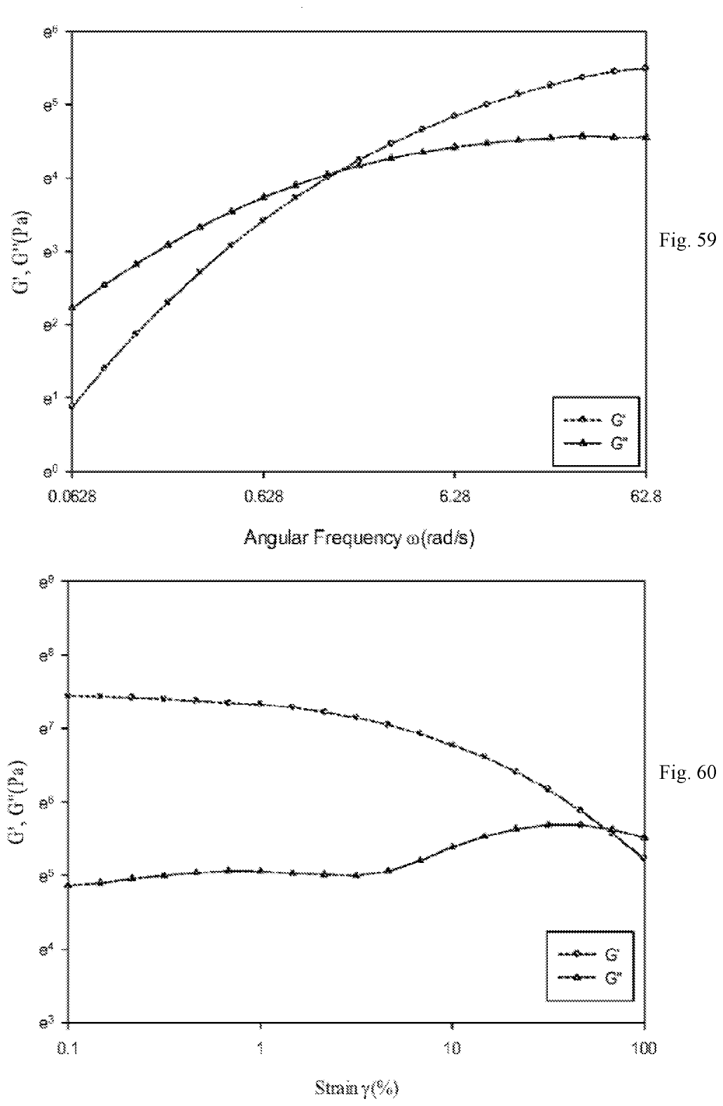

It can be known by observing time sweep in FIG. 57 that G′ of the “1.5% HA¹⁵⁰+0.1% GDGE” sample is greater than G″, indicating that this sample is a hydrogel. It can be discovered through frequency sweep in FIG. 58 that no matter how frequency changes, G′ is always greater than G″, indicating that the covalently cross-linked hyaluronic acid hydrogel prepared in example 57 is a chemically cross-linked hydrogel. It can be known through frequency sweep in FIG. 59 that with a decrease in frequency, the “1.5% HA¹⁵⁰+0.1% GDGE” sample is changed from the state of “G′ is greater than G“ ” into the state of “G′ is less than G”, indicating that the “1.5% HA¹⁵⁰+0.1% GDGE” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Meanwhile, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. It can be seen from strain sweep in FIG. 60 that under the condition of fixed frequency and temperature, as shearing force is gradually increased, the storage modulus of the covalently cross-linked hyaluronic acid hydrogel prepared in example 57 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is ordinary.

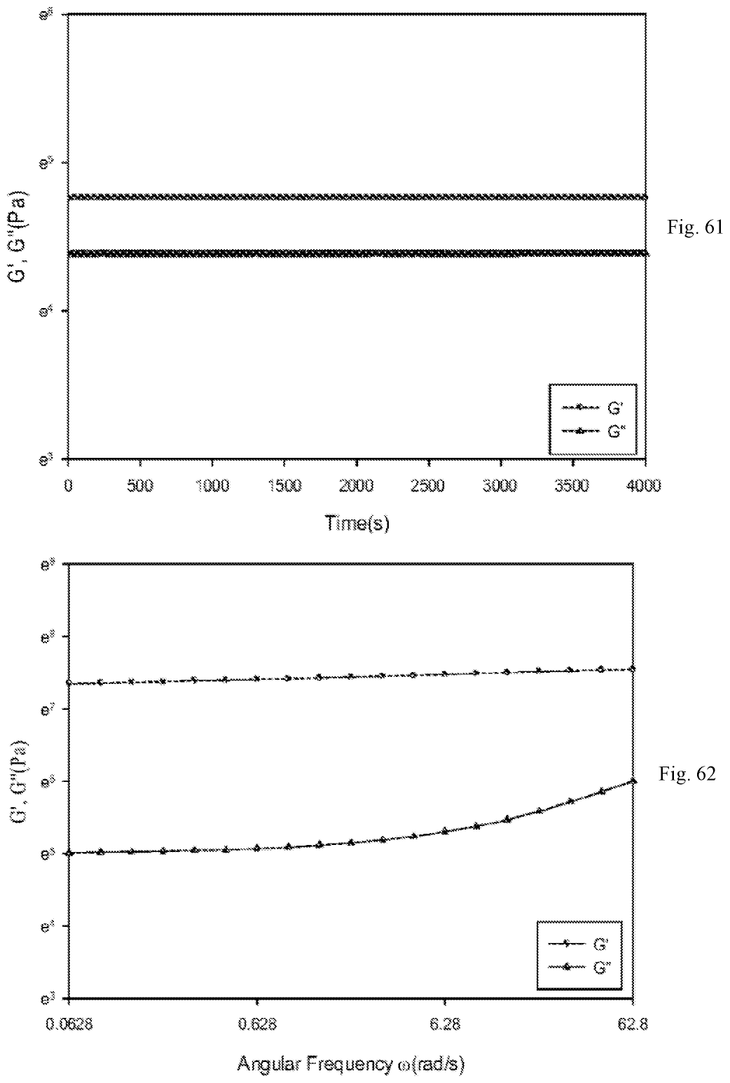

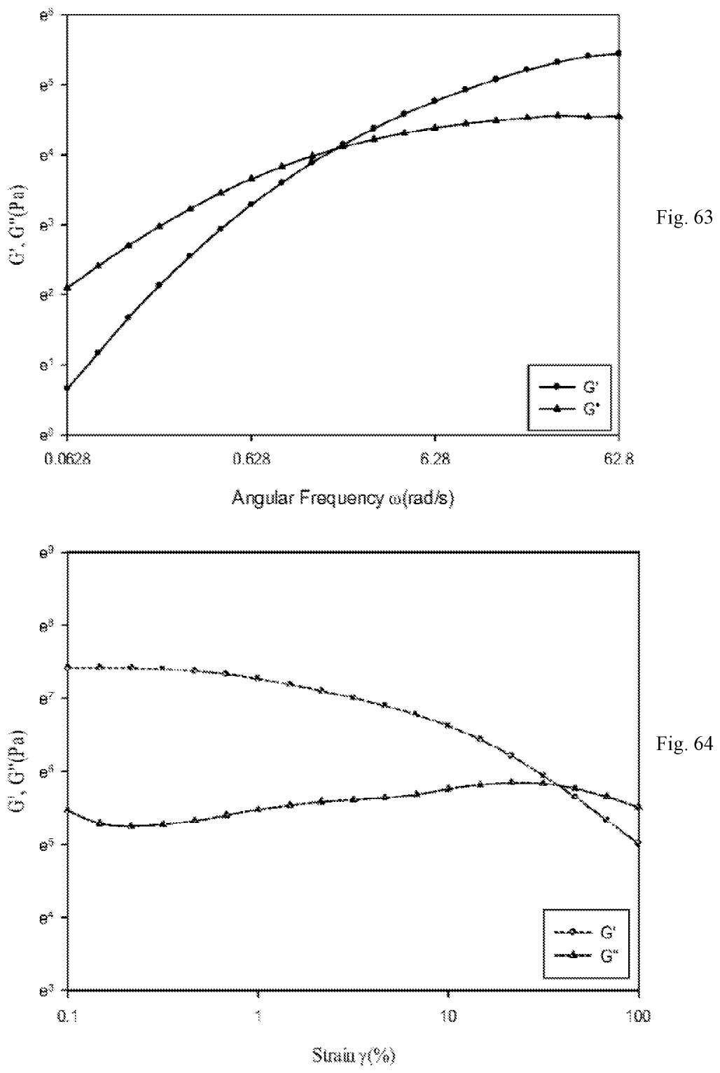

It can be known by observing time sweep in FIG. 61 that G′ of the “1.5% HA¹⁵⁰+0.2% GDGE” sample is greater than G″, indicating that this sample is a hydrogel. It can be discovered through frequency sweep in FIG. 62 that no matter how frequency changes, G′ is always greater than G″, indicating that the covalently cross-linked hyaluronic acid hydrogel prepared in example 58 is a chemically cross-linked hydrogel. It can be known through frequency sweep in FIG. 63 that with a decrease in frequency, the “1.5% HA¹⁵⁰+0.2% GDGE” sample is changed from the state of “G′ is greater than G“ ” into the state of “G′ is less than G”, indicating that the “1.5% HA¹⁵⁰+0.2% GDGE” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Meanwhile, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. It can be seen from strain sweep in FIG. 64 that under the condition of fixed frequency and temperature, as shearing force is gradually increased, the storage modulus of the covalently cross-linked hyaluronic acid hydrogel prepared in example 58 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is ordinary.

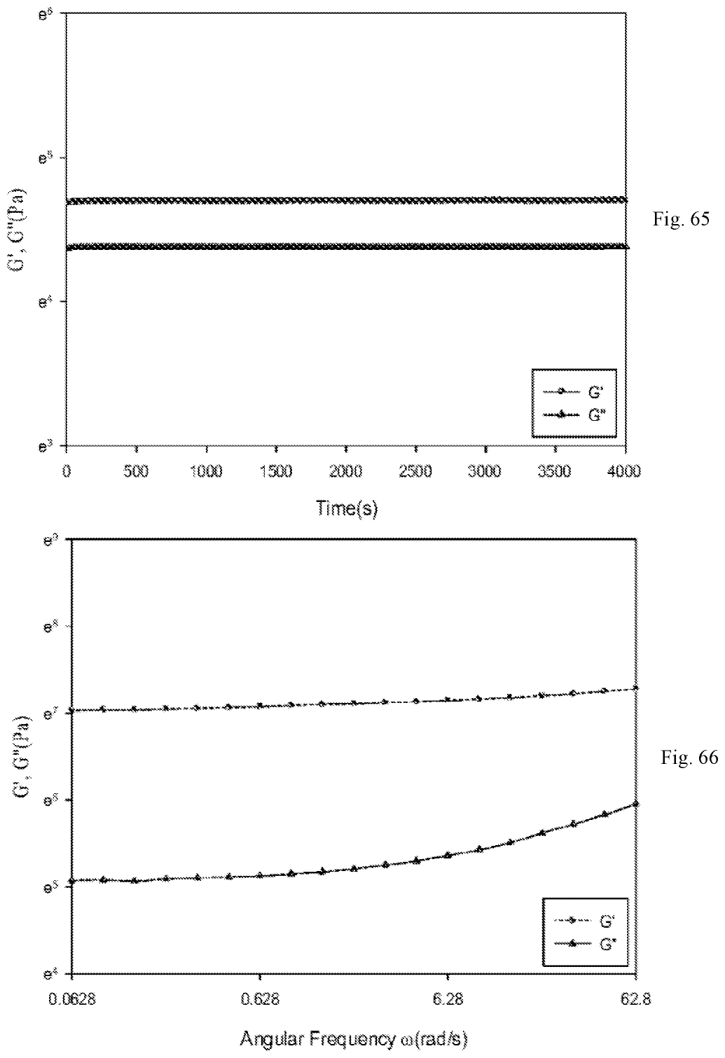

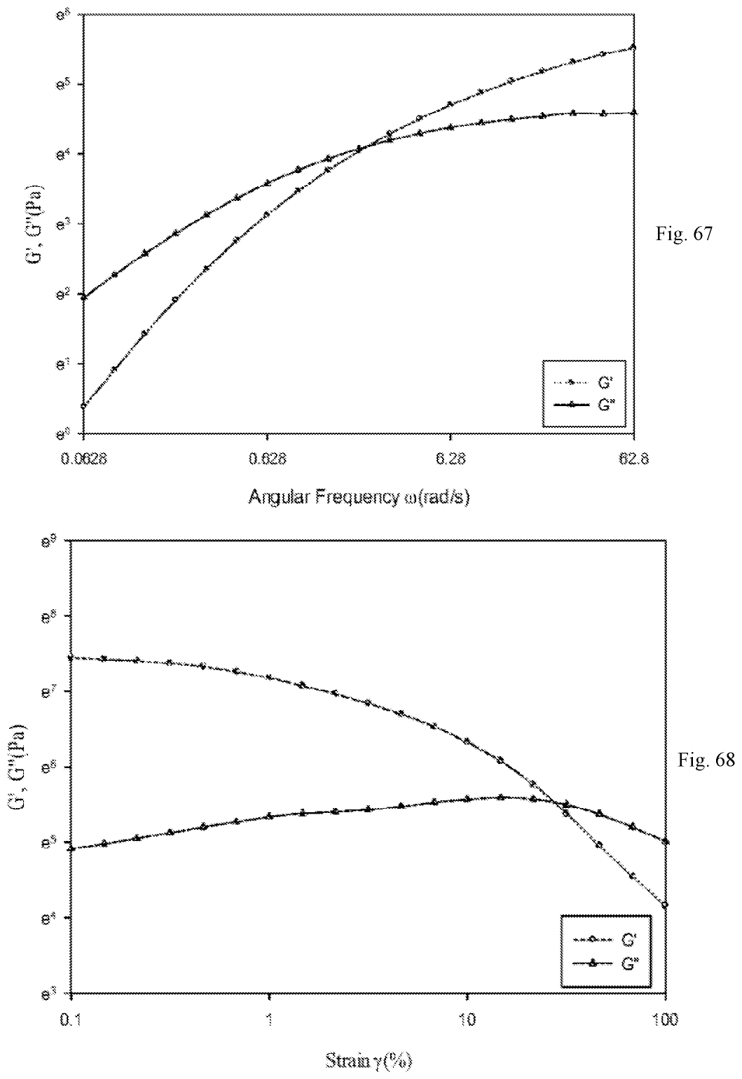

It can be known by observing time sweep in FIG. 65 that G′ of the “1.5% HA¹⁵⁰+0.4% GDGE” sample is greater than G″, indicating that this sample is a hydrogel. It can be discovered through frequency sweep in FIG. 66 that no matter how frequency changes, G′ is always greater than G″, indicating that the covalently cross-linked hyaluronic acid hydrogel prepared in example 59 is a chemically cross-linked hydrogel. It can be known through frequency sweep in FIG. 67 that with a decrease in frequency, the “1.5% HA¹⁵⁰+0.4% GDGE” sample is changed from the state of “G′ is greater than G“ ” into the state of “G′ is less than G”, indicating that the “1.5% HA¹⁵⁰+0.4% GDGE” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Meanwhile, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. Strain sweep in FIG. 68 indicates that under the condition of fixed frequency and temperature, as shearing force is gradually increased, the storage modulus of the covalently cross-linked hyaluronic acid hydrogel prepared in example 59 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is ordinary.

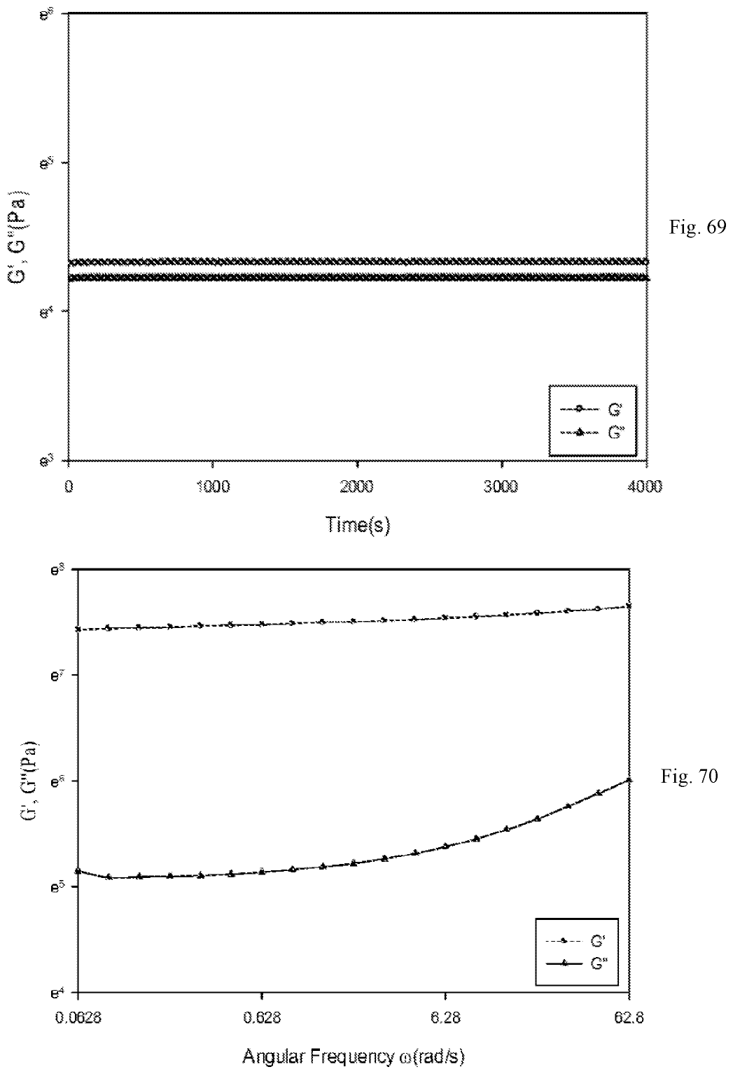

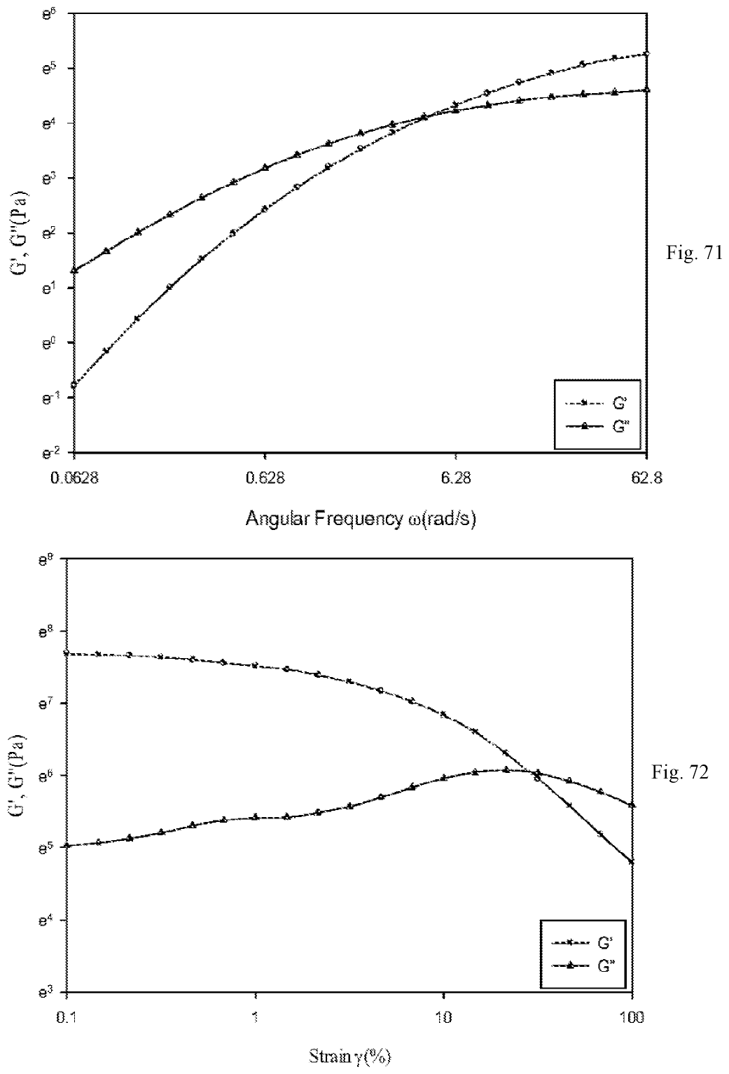

It can be known by observing time sweep in FIG. 69 that G′ of the “1.5% HA¹⁵⁰+1.0% GDGE” sample is greater than G″, indicating that this sample is a hydrogel. It can be discovered through frequency sweep in FIG. 70 that no matter how frequency changes, G′ is always greater than G″, indicating that the covalently cross-linked hyaluronic acid hydrogel prepared in example 60 is a chemically cross-linked hydrogel. It can be known through frequency sweep in FIG. 71 that with a decrease in frequency, the “1.5% HA¹⁵⁰+1.0% GDGE” sample is changed from the state of “G′ is greater than G“ ” into the state of “G′ is less than G”, indicating that the “1.5% HA¹⁵⁰+1.0% GDGE” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Meanwhile, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. Strain sweep in FIG. 72 indicates that under the condition of fixed frequency and temperature, as shearing force is gradually increased, the storage modulus of the covalently cross-linked hyaluronic acid hydrogel prepared in example 60 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is ordinary.

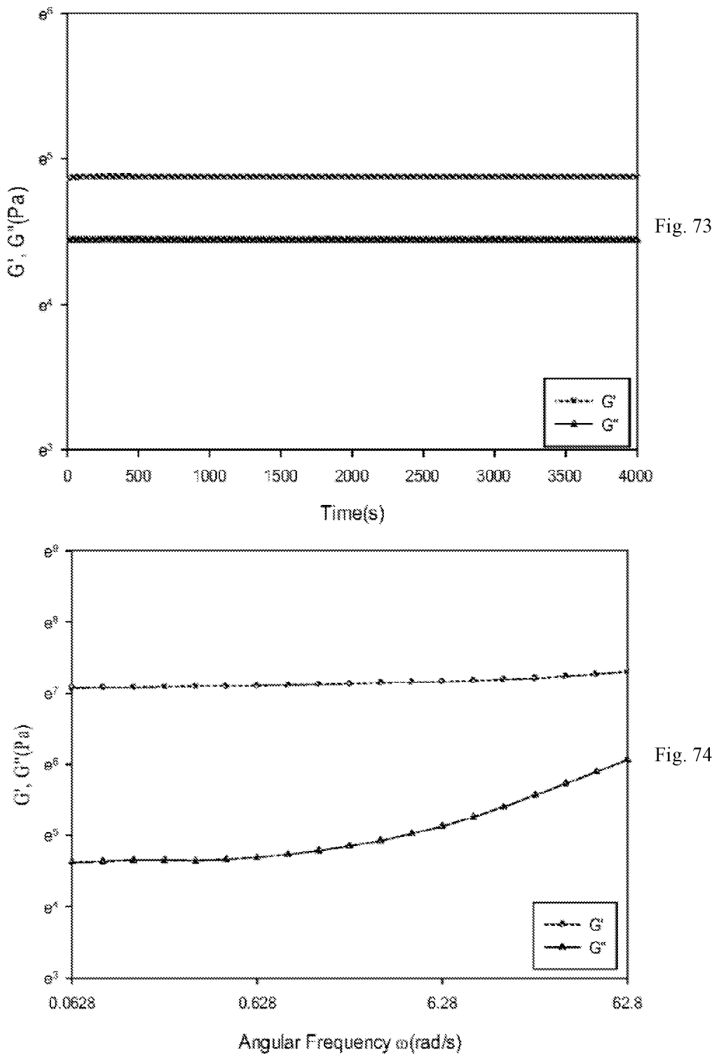

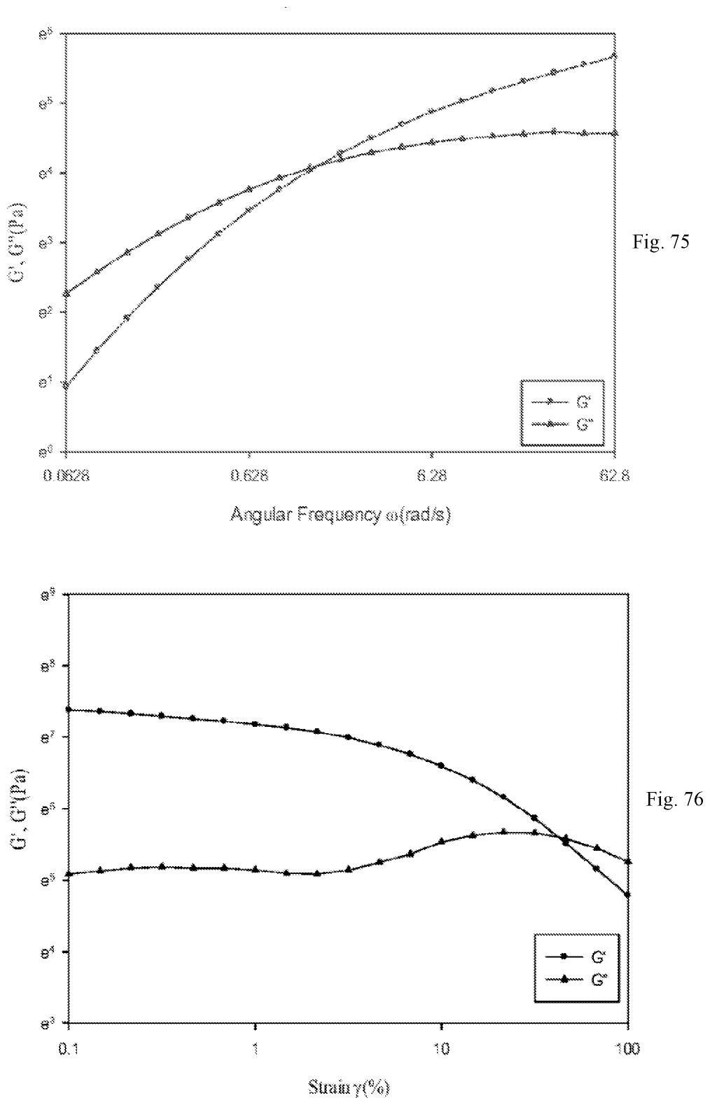

It can be known by observing time sweep in FIG. 73 that G′ of the “1.5% HA¹⁵⁰+0.1% PEG500” sample is greater than G″, indicating that this sample is a hydrogel. It can be discovered through frequency sweep in FIG. 74 that no matter how frequency changes, G′ is always greater than G″, indicating that the covalently cross-linked hyaluronic acid hydrogel prepared in example 69 is a chemically cross-linked hydrogel. It can be known through frequency sweep in FIG. 75 that with a decrease in frequency, the “1.5% HA¹⁵⁰+0.1% PEG500” sample is changed from the state of “G′ is greater than G“ ” into the state of “G′ is less than G”, indicating that the “1.5% HA¹⁵⁰+0.1% PEG500” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Moreover, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. It can be seen through strain sweep in FIG. 76 that under the condition of fixed frequency and temperature, as shearing force is gradually increased, the storage modulus of the covalently cross-linked hyaluronic acid hydrogel prepared in example 69 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is ordinary.

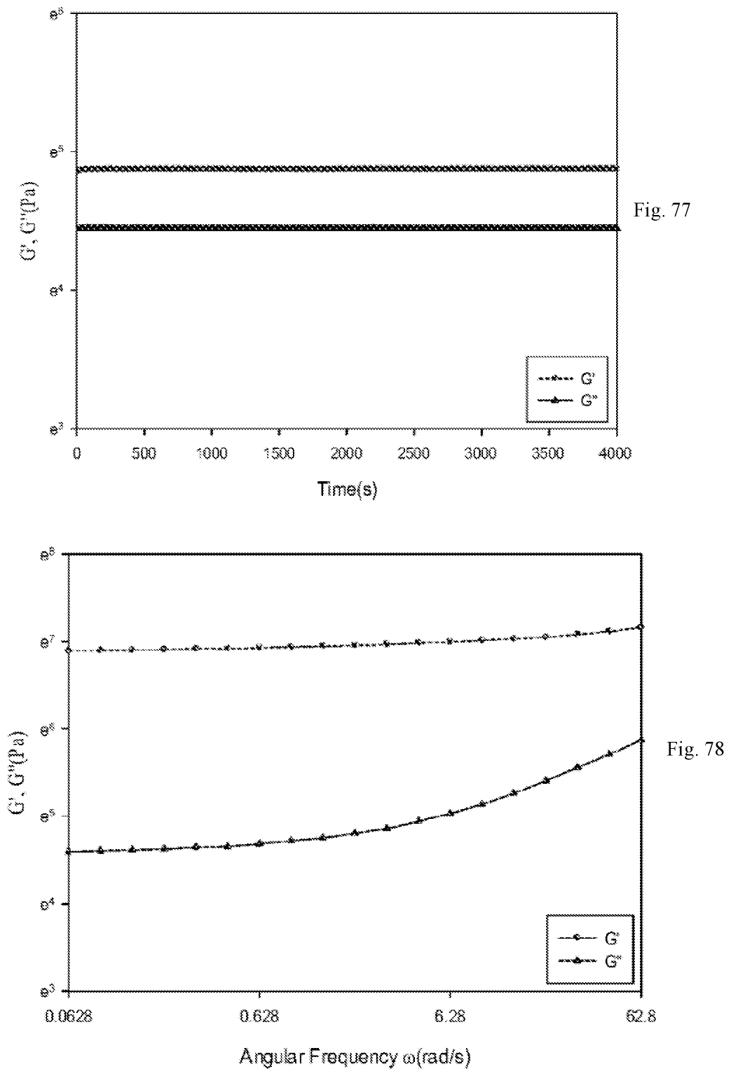

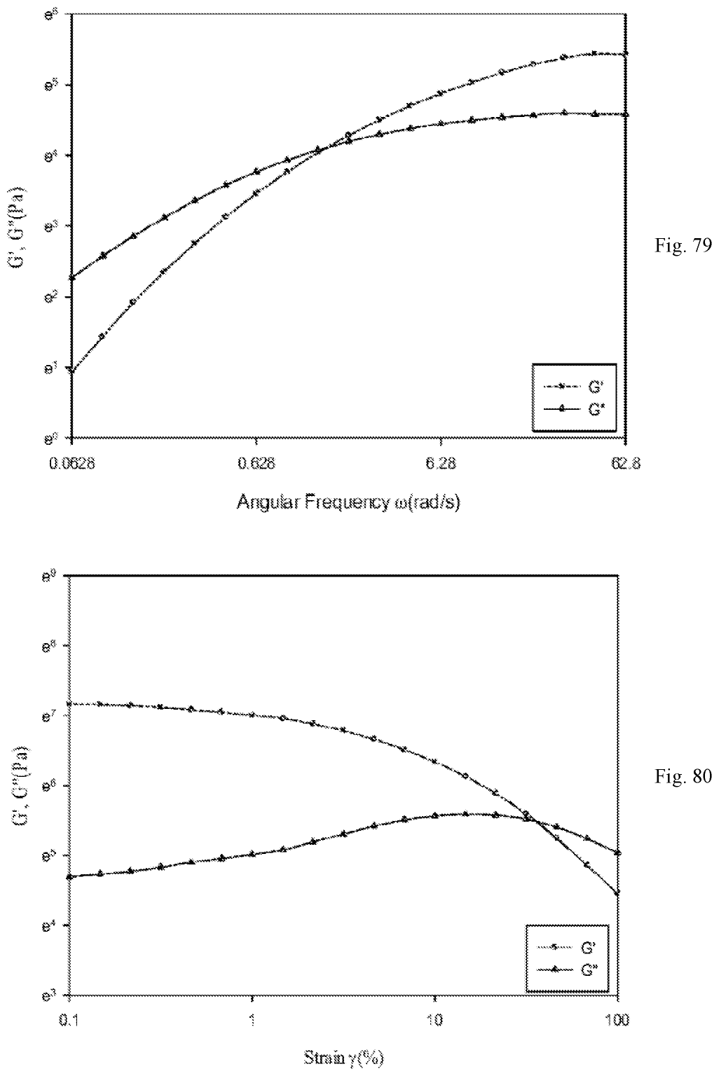

It can be known by observing time sweep in FIG. 77 that G′ of the “1.5% HA¹⁵⁰+0.2% PEG500” sample is greater than G″, indicating that this sample is a hydrogel. It can be discovered through frequency sweep in FIG. 78 that no matter how frequency changes, G′ is always greater than G″, indicating that the covalently cross-linked hyaluronic acid hydrogel prepared in example 70 is a chemically cross-linked hydrogel. It can be known through frequency sweep in FIG. 79 that with a decrease in frequency, the “1.5% HA¹⁵⁰+0.2% PEG500” sample is changed from the state of “G′ is greater than G“ ” into the state of “G′ is less than G”, indicating that the “1.5% HA¹⁵⁰+0.2% PEG500” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Meanwhile, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. It can be seen through strain sweep in FIG. 80 indicates that under the condition of fixed frequency and temperature, as shearing force is gradually increased, the storage modulus of the covalently cross-linked hyaluronic acid hydrogel prepared in example 70 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is ordinary.

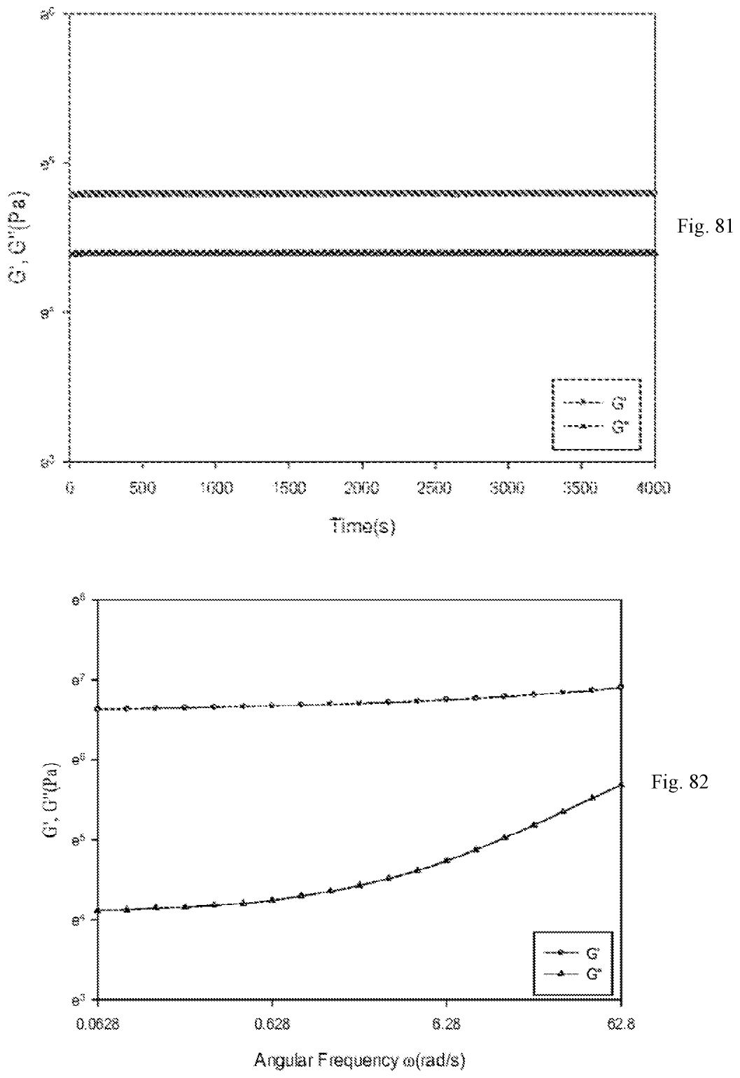

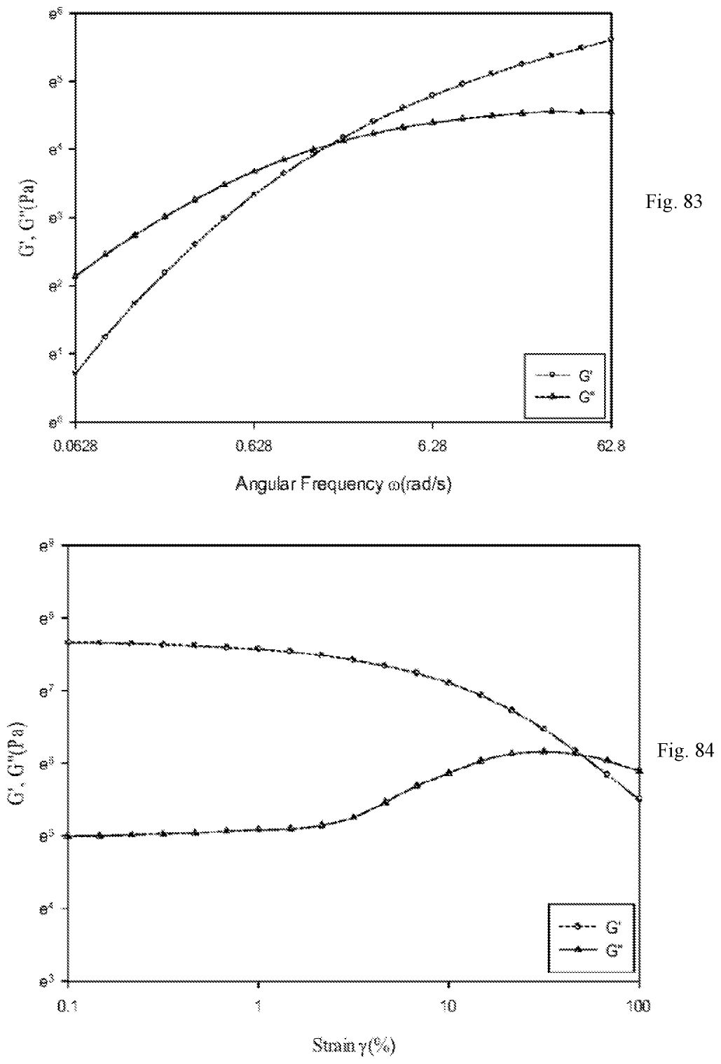

It can be known by observing time sweep in FIG. 81 that G′ of the “1.5% HA¹⁵⁰+0.4% PEG500” sample is greater than G″, indicating that this sample is a hydrogel. It can be discovered through frequency sweep in FIG. 82 that no matter how frequency changes, G′ is always greater than G″, indicating that the covalently cross-linked hyaluronic acid hydrogel prepared in example 71 is a chemically cross-linked hydrogel. It can be known through frequency sweep in FIG. 83 that with a decrease in frequency, the “1.5% HA¹⁵⁰+0.4% PEG500” sample is changed from the state of “G′ is greater than G“ ” into the state of “G′ is less than G”, indicating that the “1.5% HA¹⁵⁰+0.4% PEG500” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Meanwhile, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. Strain sweep in FIG. 84 indicates that under the condition of fixed frequency and temperature, as shearing force is gradually increased, the storage modulus of the covalently cross-linked hyaluronic acid hydrogel prepared in example 71 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is ordinary.

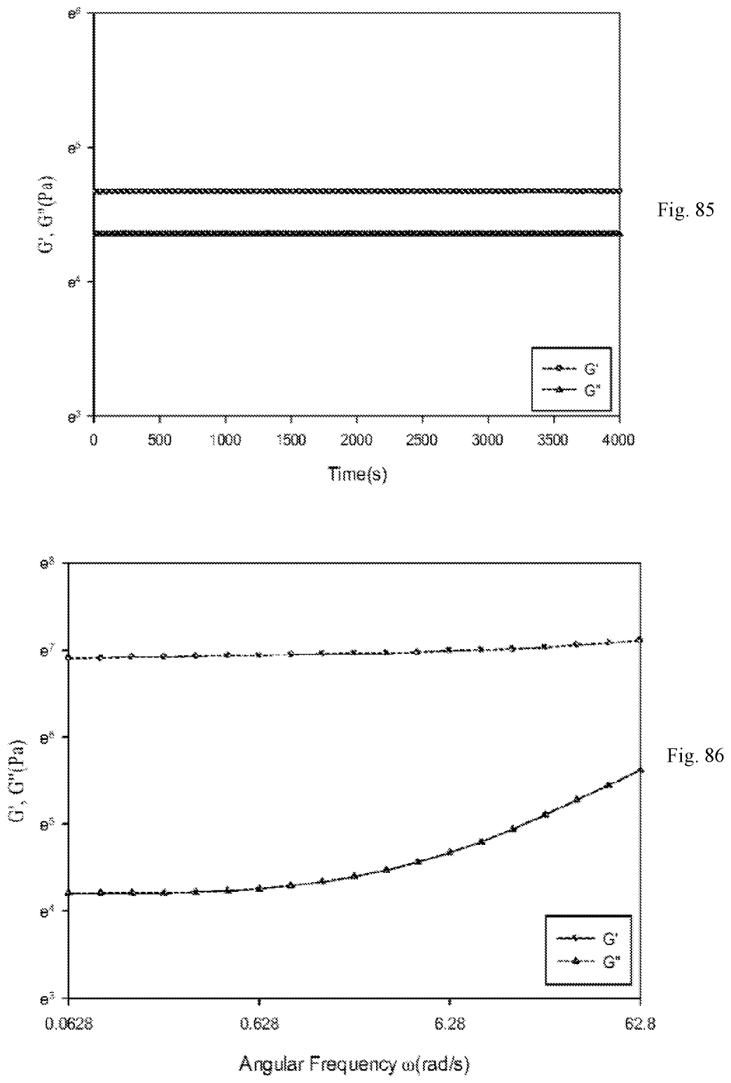

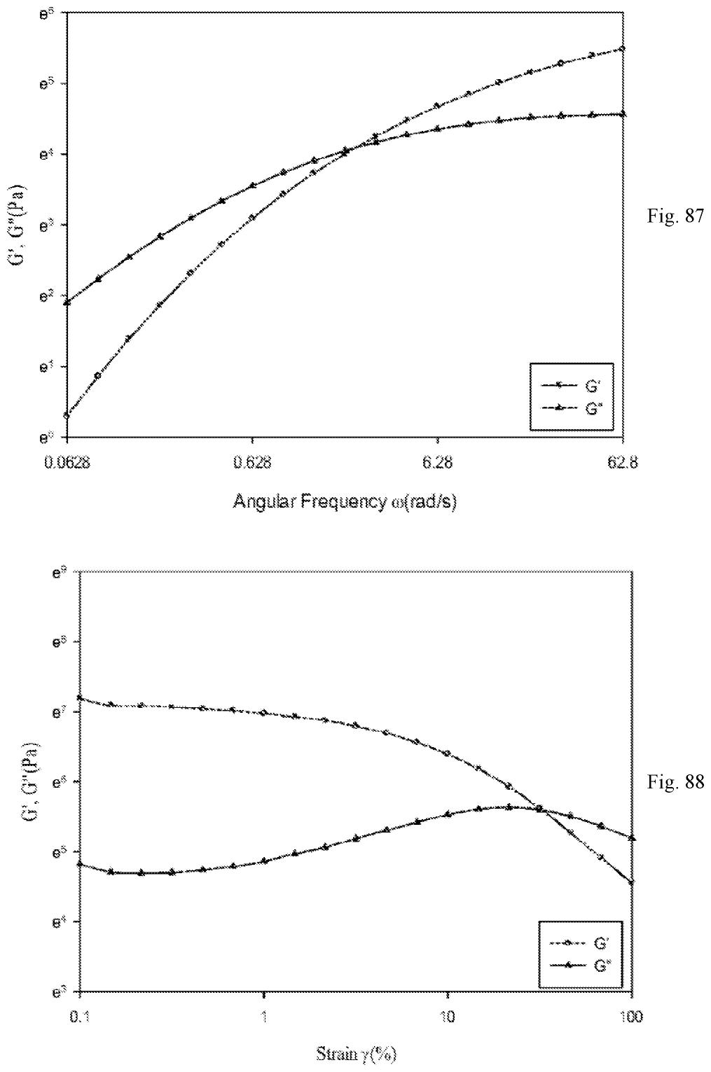

It can be known by observing time sweep in FIG. 85 that G′ of the “1.5% HA¹⁵⁰+1.0% PEG500” sample is greater than G″, indicating that this sample is a hydrogel. It can be discovered through frequency sweep in FIG. 86 that no matter how frequency changes, G′ is always greater than G″, indicating that the covalently cross-linked hyaluronic acid hydrogel prepared in example 72 is a chemically cross-linked hydrogel. It can be known through frequency sweep in FIG. 87 that with a decrease in frequency, the “1.5% HA¹⁵⁰+1.0% PEG500” sample is changed from the state of “G′ is greater than G“ ” into the state of “G′ is less than G”, indicating that the “1.5% HA¹⁵⁰+1.0% PEG500” sample is a physically cross-linked hydrogel, and this is because HA itself contains hydroxy and carboxyl. When HA is dissolved in purified water, hyaluronic acid itself has hydrogen bonds formed in molecules, and can also form intermolecular hydrogen bonds with water molecules, and the hydrogen bonds and van der Waals' force formed between macromolecular chains lead to physical crosslinking, forming a physical hydrogel with a network structure. With an increase in frequency, the physically cross-linked hydrogel structure will be destroyed in the end, so that G′ is less than G″. Meanwhile, it can also indicate that the physically cross-linked hydrogel will not be turned into a chemically cross-linked hydrogel due to 72 hours of standing, proving that the present invention promotes the chemical crosslinking between the crosslinking agent and hyaluronic acid through the freeze-drying technique. It can be seen through strain sweep in FIG. 88 indicates that under the condition of fixed frequency and temperature, as shearing force is gradually increased, the storage modulus of the covalently cross-linked hyaluronic acid hydrogel prepared in example 72 changes and begins to drop, which indicates that the network structure of the hydrogel has begun to be destroyed under this strain. When G′ is less than G″, it indicates that the network structure of the hydrogel has been thoroughly destroyed, indicating that the viscoelasticity of this hydrogel is ordinary.

### Example 76: Experiment on Degradation of Hyaluronic Acid Hydrogels in Plasma

The covalently cross-linked hyaluronic acid aerogels prepared in examples 9-12, 21-24, 33-36, 45-48, 57-60 and 69-72 were respectively taken, cut into the size of 0.5 cm×0.5 cm and soaked in 3 mL of PBS buffer solution to stand for 30 minutes, so that the covalently cross-linked hyaluronic acid aerogels absorbed liquid and swelled to obtain the covalently cross-linked hyaluronic acid hydrogels. The PBS buffer solution in the culture dishes and attached to the surface of the hydrogels was completely sucked using filter paper and a disposable straw, and the weights of the hydrogels were measured and recorded as Wₒ (g). The hydrogels were then completely soaked in plasma and put into a 37° C. biochemical incubator to stand. Weights were measured once every 8 hours. Weights were measured and recorded as Wₜ (g) until the hydrogels were completely degraded. The experiment was repeated three times.

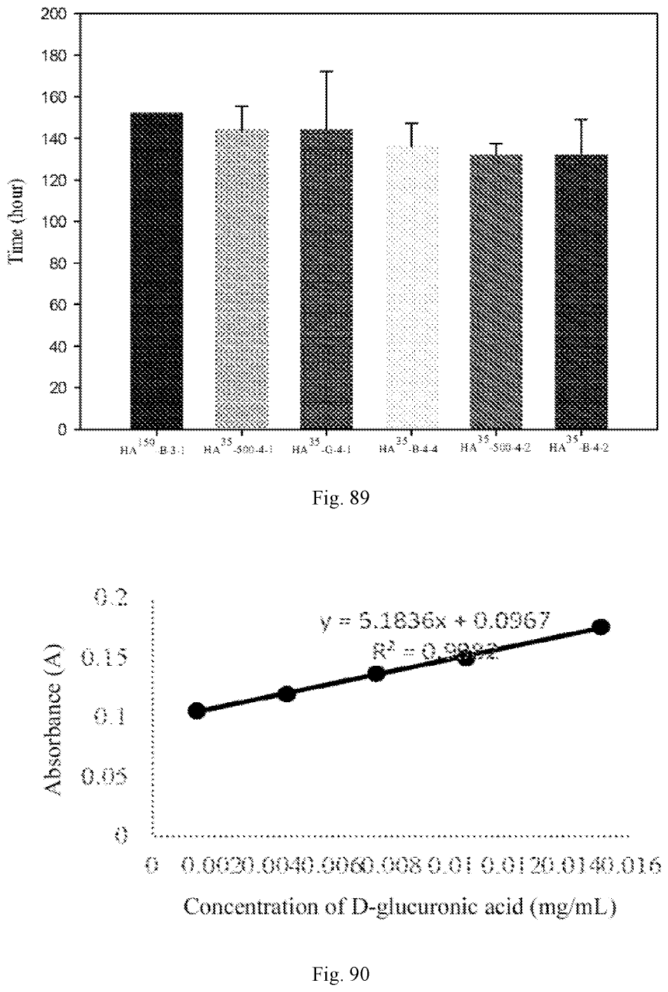

The experimental results are shown in FIG. 89. By detecting the degradabilities of the covalently cross-linked hyaluronic acid hydrogels in plasma, their stabilities in plasma were judged and analyzed. Among them, the covalently cross-linked hyaluronic acid hydrogel (sample name: HA¹⁵⁰-B-3-1) prepared in example 45 was completely degraded in 152 hours; the covalently cross-linked hyaluronic acid hydrogel (sample name: HA³⁵-P500-4-1) prepared in example 33 and the covalently cross-linked hyaluronic acid hydrogel (sample name: HA³⁵-G-4-1) prepared in example 21 were completely degraded in 144 hours; and this indicates that the prepared covalently cross-linked hyaluronic acid hydrogels have good stabilities in plasma.

### Example 77: Experiment on In-Vitro Enzymatic Degradation of Hyaluronic Acid Hydrogels

1. Drawing of D-glucuronic acid standard curve: 1.5 mg/mL of D-glucuronic acid solution was prepared, and 0.5 mL of the D-glucuronic acid solution was then taken and diluted to 50 mL with water to prepare a D-glucuronic acid standard solution. This standard solution was respectively diluted to different concentrations, which are respectively 0.0015 mg/mL, 0.0045 mg/mL, 0.0075 mg/mL, 0.0105 mg/mL and 0.0150 mg/mL. 60 μL of solution to be detected was transferred into each centrifuge tube and put into ice water bath. 300 μL of 0.025 mol/L solution of borax in sulfuric acid was slowly added into each tube, and after addition, uniform mixing was performed. The centrifuge tubes were then heated in 100° C. water bath for 15 minutes, taken out and put into ice water bath until they were cooled to room temperature. 12 μL of solution of carbazole in ethanol was added into each tube, and uniform mixing was performed after addition. The centrifuge tubes were heated in 100° C. water bath for 15 minutes, taken out and then cooled to room temperature. A microplate reader was used to detect absorbances at 530 nm. According to the detected absorbances and the known concentrations of the D-glucuronic acid solution, a D-glucuronic acid standard curve was drawn.

The D-glucuronic acid standard curve is shown in FIG. 90.

2. The covalently cross-linked hyaluronic acid aerogels prepared in examples 9-12, 21-24, 33-36, 45-48, 57-60 and 69-72 and a freeze-dried hyaluronic acid sample without crosslinking agent as a control were respectively taken. The experimental samples were grouped as shown in Table 4.

They were then respectively cut into the size of 0.5 cm×0.5 cm and soaked in 3 mL of PBS buffer solution to stand for 30 minutes, so that the covalently cross-linked hyaluronic acid aerogels absorbed liquid and swelled to obtain covalently cross-linked hyaluronic acid hydrogels, while the freeze-dried hyaluronic acid sample without crosslinking agent also absorbed liquid and swelled. The PBS buffer solution attached to the surface of the covalently cross-linked hyaluronic acid hydrogels and the PBS buffer solution attached to the surface of the freeze-dried hyaluronic acid sample without crosslinking agent were completely sucked using filter paper and a disposable straw. The covalently cross-linked hyaluronic acid hydrogels and the freeze-dried hyaluronic acid sample without crosslinking agent were soaked in 3 mL of hyaluronidase solution (10 U/mL) to undergo an enzymatic degradation experiment. With the time of putting the samples into the hyaluronidase solution being taken as a starting time, 60 μL of supernatant was taken as liquid to be detected every 24 hours, and 60 μL of fresh hyaluronidase solution (10 U/mL) was replenished; and 60 μL of liquid to be detected was transferred into each centrifuge tube and put into ice water bath. 300 μL of 0.025 mol/L solution of borax in sulfuric acid was slowly added into each tube, and after addition, uniform mixing was performed. The centrifuge tubes were then heated in 100° C. water bath for 15 minutes, taken out and put into ice water bath until they were cooled to room temperature. 12 μL of solution of carbazole in ethanol was added into each tube, and uniform mixing was performed after addition. The centrifuge tubes were heated in 100° C. water bath for 15 minutes, taken out and then cooled to room temperature. A microplate reader was used to detect absorbances at 530 nm. The degradation percentages of the covalently cross-linked hyaluronic acid hydrogels were calculated according to the D-glucuronic acid standard curve.

TABLE 4
Experimental Samples and Specific Formulas

| Example | Sample Name |
|---|---|
| 9 | HA³⁵-B-4-1 |
| 10 | HA³⁵-B-4-2 |
| 11 | HA³⁵-B-4-3 |
| 12 | HA³⁵-B-4-4 |
| 21 | HA³⁵-G-4-1 |
| 22 | HA³⁵-G-4-2 |
| 23 | HA³⁵-G-4-3 |
| 24 | HA³⁵-G-4-4 |
| 33 | HA³⁵-P500-4-1 |
| 34 | HA³⁵-P500-4-2 |
| 35 | HA³⁵-P500-4-3 |
| 36 | HA³⁵-P500-4-4 |
| 45 | HA¹⁵⁰-B-3-1 |
| 46 | HA¹⁵⁰-B-3-2 |
| 47 | HA¹⁵⁰-B-3-3 |
| 48 | HA¹⁵⁰-B-3-4 |
| 57 | HA¹⁵⁰-G-3-1 |
| 58 | HA¹⁵⁰-G-3-2 |
| 59 | HA¹⁵⁰-G-3-3 |
| 60 | HA¹⁵⁰-G-3-4 |
| 69 | HA¹⁵⁰-P500-3-1 |
| 70 | HA¹⁵⁰-P500-3-2 |
| 71 | HA¹⁵⁰-P500-3-3 |
| 72 | HA¹⁵⁰-P500-3-4 |
| \ | 4% HA³⁵ |
| \ | 1.5% HA¹⁵⁰ |

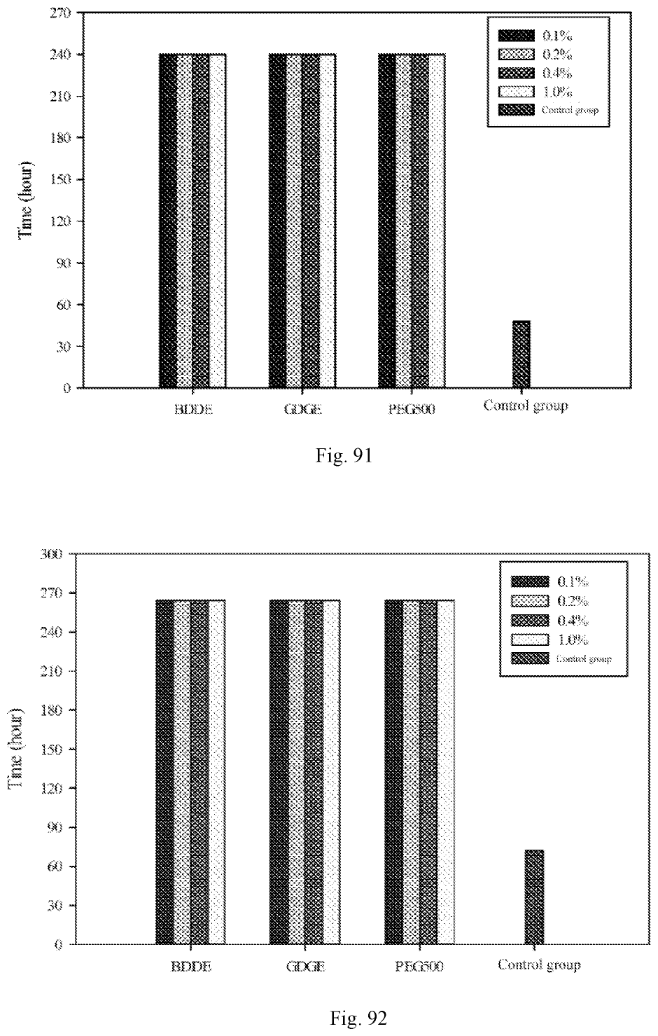

The experimental results are shown in FIG. 91 and FIG. 92. The enzymatic degradation resistance of the hydrogels prepared in chemical crosslinking reaction is far better than that of the non-cross-linked hyaluronic acid sample. Moreover, when the molecular weight and concentration of hyaluronic acid are the same, the in-vitro enzymatic degradation times of the covalently cross-linked hyaluronic acid hydrogels prepared with different crosslinking agents at different crosslinking agent doses are equal.

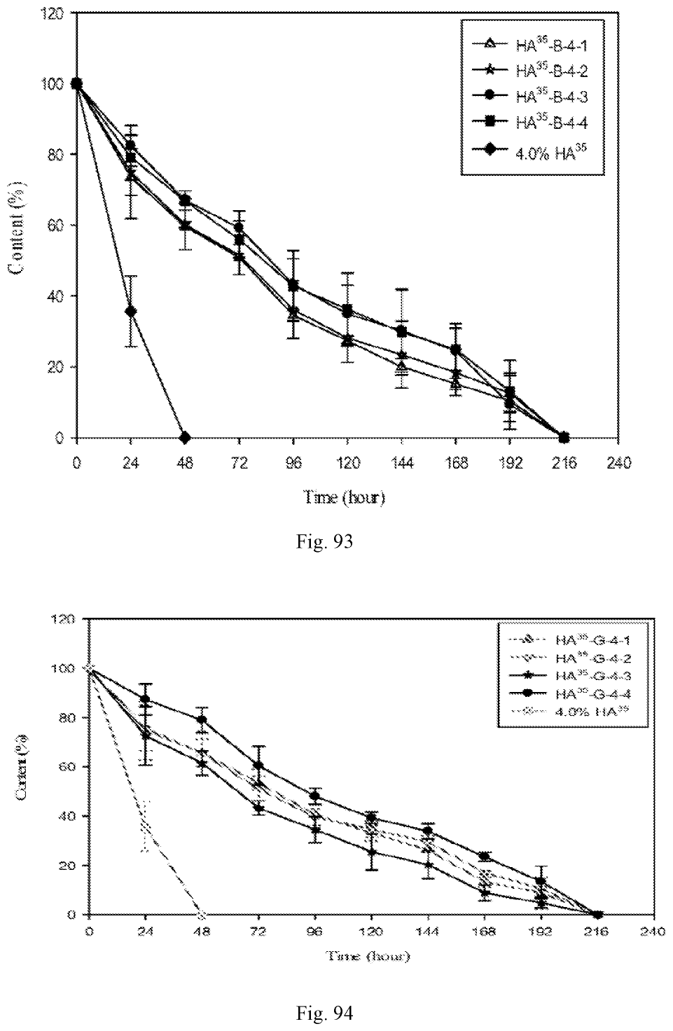

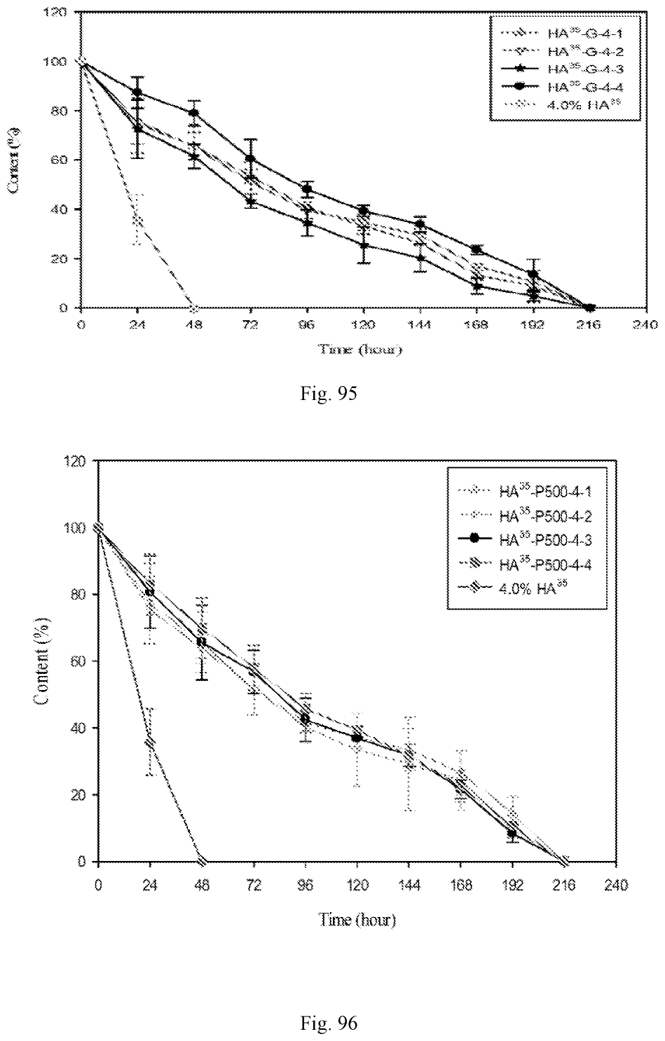

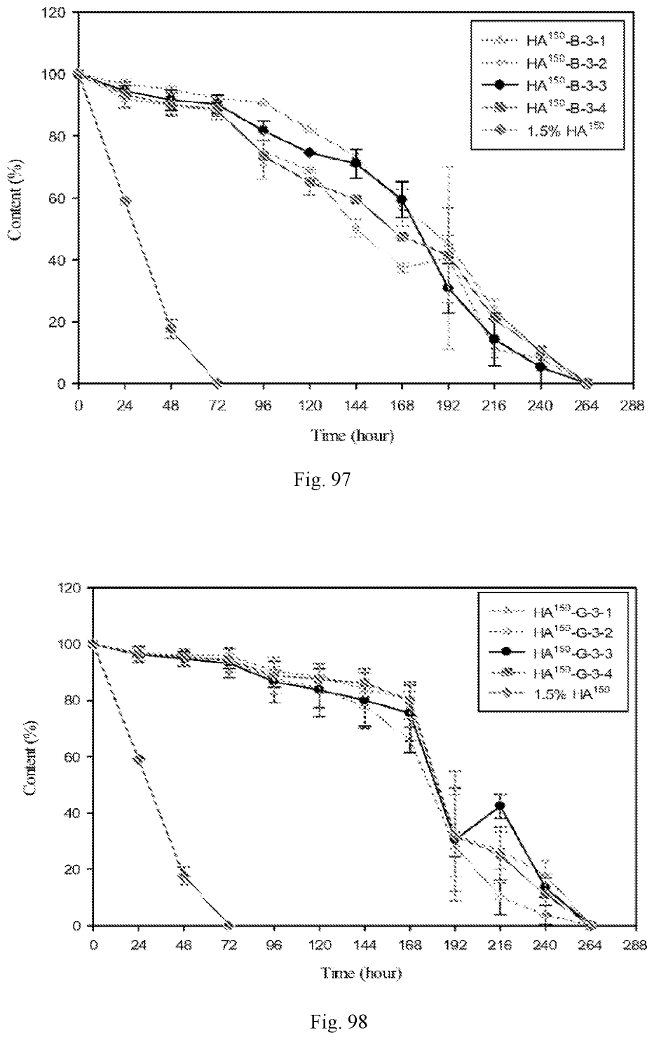

It can be discovered in FIGS. 93-98 that hyaluronidase will gradually degrade the hyaluronic acid hydrogels. Compared with the non-cross-linked hyaluronic acid sample, the covalently cross-linked hyaluronic acid hydrogels prepared in the invention have excellent enzymatic degradation resistance. This is because the chemical modification and crosslinking of hyaluronic acid can increase the enzymatic degradation resistance of the hyaluronic acid hydrogels, thus prolonging their in-vitro enzymatic degradation times.

The experimental results above indicate that the covalently cross-linked hyaluronic acid aerogels have excellent liquid-absorbing property, and can quickly absorb water and swell to obtain the covalently cross-linked hyaluronic acid hydrogels. Through the experiments on the degradation of the covalently cross-linked hyaluronic acid hydrogels in PBS buffer solution, plasma and hyaluronidase solution and the rheological experiment on the covalently cross-linked hyaluronic acid hydrogels, the following conclusions can be drawn: the covalently cross-linked hyaluronic acid hydrogels prepared in the present invention carry out chemical crosslinking reaction under the condition of freeze-drying, and the prepared hydrogels have good stability and enzymatic degradation resistance. The covalently cross-linked hyaluronic acid hydrogels have a good application prospect in the fields of tissue engineering and medical materials.

### Example 78: Cytotoxicity Experiment and Cytofluorescent Experiment on Hyaluronic Acid Hydrogels

(1) Cytotoxicity Experiment

\textcircled{1} Grouping of Experimental Examples

TABLE 5
Experimental Samples and Specific Formulas

| | | Molecular | Concentration | | Concentration |
|---|---|---|---|---|---|
| | | Weight of | of Hyaluronic | | of Crosslinking |
| Serial | | Hyaluronic | Acid | Crosslinking | Agent |
| Number | Sample | Acid/Da | (w/v, %) | Agent | (v/v, %) |
| 1 | HA³⁵-B-4-1 | 3.5 × 10⁵ | 4.0 | BDDE | 0.1 |
| 2 | HA³⁵-B-4-2 | 3.5 × 10⁵ | 4.0 | BDDE | 0.2 |
| 3 | HA³⁵-B-4-3 | 3.5 × 10⁵ | 4.0 | BDDE | 0.4 |
| 4 | HA³⁵-B-4-4 | 3.5 × 10⁵ | 4.0 | BDDE | 1.0 |
| 5 | HA³⁵-G-4-1 | 3.5 × 10⁵ | 4.0 | GDGE | 0.1 |
| 6 | HA³⁵-G-4-2 | 3.5 × 10⁵ | 4.0 | GDGE | 0.2 |
| 7 | HA³⁵-G-4-3 | 3.5 × 10⁵ | 4.0 | GDGE | 0.4 |
| 8 | HA³⁵-G-4-4 | 3.5 × 10⁵ | 4.0 | GDGE | 1.0 |
| 9 | HA³⁵-P500-4-1 | 1.5 × 10⁶ | 4.0 | PEG500 | 0.1 |
| 10 | HA³⁵-P500-4-2 | 1.5 × 10⁶ | 4.0 | PEG500 | 0.2 |
| 11 | HA³⁵-P500-4-3 | 1.5 × 10⁶ | 4.0 | PEG500 | 0.4 |
| 12 | HA³⁵-P500-4-4 | 1.5 × 10⁶ | 4.0 | PEG500 | 1.0 |
| 13 | HA¹⁵⁰-B-3-1 | 1.5 × 10⁶ | 1.5 | BDDE | 0.1 |
| 14 | HA¹⁵⁰-B-3-2 | 1.5 × 10⁶ | 1.5 | BDDE | 0.2 |
| 15 | HA¹⁵⁰-B-3-3 | 1.5 × 10⁶ | 1.5 | BDDE | 0.4 |
| 16 | HA¹⁵⁰-B-3-4 | 1.5 × 10⁶ | 1.5 | BDDE | 1.0 |
| 17 | HA¹⁵⁰-G-3-1 | 1.5 × 10⁶ | 1.5 | GDGE | 0.1 |
| 18 | HA¹⁵⁰-G-3-2 | 1.5 × 10⁶ | 1.5 | GDGE | 0.2 |
| 19 | HA¹⁵⁰-G-3-3 | 1.5 × 10⁶ | 1.5 | GDGE | 0.4 |
| 20 | HA¹⁵⁰-G-3-4 | 1.5 × 10⁶ | 1.5 | GDGE | 1.0 |
| 21 | HA¹⁵⁰-P500-3-1 | 1.5 × 10⁶ | 1.5 | PEG500 | 0.1 |
| 22 | HA¹⁵⁰-P500-3-2 | 1.5 × 10⁶ | 1.5 | PEG500 | 0.2 |
| 23 | HA¹⁵⁰-P500-3-3 | 1.5 × 10⁶ | 1.5 | PEG500 | 0.4 |
| 24 | HA¹⁵⁰-P500-3-4 | 1.5 × 10⁶ | 1.5 | PEG500 | 1.0 |

\textcircled{2} Co-Culture of Hydrogels and Cells

a. L929 mouse fibroblasts were subcultured.

b. A blank group was set, in which L929 mouse fibroblasts were cultured with complete medium; a positive control group was set, in which L929 mouse fibroblasts were cultured with 0.64% phenol; and a sample group was set, in which different hydrogels were respectively cultured with L929 mouse fibroblasts under the condition of complete medium.

c. Various aerogels No. 1 to No. 24 were soaked at a certain weight in PBS buffer solution for 24 hours, so that aerogels absorbed water and swelled to hydrogels. The PBS buffer solution attached to the surface of the hydrogels was then completely sucked with filter paper, and the hydrogels were put into 96-well plates. An ultraviolet lamp was used to irradiate the 96-well plates for 30 minutes, wherein each type of hydrogel required three samples as controls.

d. A fifth passage of L929 mouse fibroblasts were collected, and the original medium was discarded. A glass dropper was used to suck a certain amount of PBS buffer solution to wash the cells twice or three times.

e. 0.25% trypsin solution was used to digest the cells, and when more than 60% of cells dropped from the culture flask, the glass dropper was used to collect cell suspensions into 15 mL centrifuge tubes.

f. The centrifuge tubes were put into a centrifuge and centrifuged at a rotational speed of 1000 rpm under room temperature for 5 minutes, and supernatant was then discarded.

g. Complete medium was added, and the glass dropper was used for blowing to prepare cell suspensions. A hemocytometer was used for counting, the cell concentration was then diluted to 1×10⁴/cm², and the glass dropper was then used for blowing multiple times, so that the cells were evenly distributed.

h. 200 μL of cell suspension was directly added into the sample group and the blank group, wherein in the sample group the cells were uniformly inoculated on the hydrogels, while in the positive control group the cells were cultured with 0.64% phenol.

i. The 96-well plates were put into a cell incubator under the conditions of 5% of CO₂ in parts by volume and 37° C. for 2 days of culture by distribution.

\textcircled{3} CCK-8 Experiment on L929 Mouse Fibroblasts

a. Principle of CCK-8 Experiment

Cell Counting Kit-8 (CCK-8 for short) is commonly used to assay cytotoxicity, cell viability and cell growth. Because the CCK-8 kit is easy to use and its assay results are accurate, the CCK-8 kit has been used widely. The principle of the CCK-8 assay method is as follows:

[2-(2-methoxy-4-nitrophenyl)-3-(4-nitrophenyl)-5-(2,4-disulfopheny)-2H-tetrazolium monosodium salt] (Water soluble tetrazolium, WST-8) contained in CCK-8 reagent is reduced into water-soluble yellow formazen dye by dehydrogenases of mitochondria of living cells under the action of an electron carrier (Li Hongyan, et al., 2005). A microplate reader was then used to determine OD values at a wavelength of 450 nm. The higher the OD values, the more the number of living cells, and cell growth rates were analyzed according to this equation (Xi Tingfei, 1999).

b. Operations of CCK-8 Experiment

After the cells in the 96-well plates were respectively cultured in the cell incubator under the conditions of 5% CO2 and 37° C. for 2 days, 20 μL of CCK-8 solution was added into the 96-well plate corresponding to each group, and in the process of adding CCK-8 assay solution, bubbles were not allowed to be produced in order to prevent absorbance value from being affected. After addition, the 96-well plates were put into the incubator to continue with incubation for 1 hour, followed by the detection of their absorbance value at 450 nm by the microplate reader. Obtaining cellular relative growth rates (RGR) in each group according to the following formula.

Cellular relative growth rate (RGR)=sample group absorbance value/control group absorbance value×100%.

According to a mean of the cellular relative growth rates of each group of samples, the cellular relative growth rates of each group of samples were described and analyzed, and the toxicities of the materials were correspondingly evaluated according to Table 6 (the Drug Administration of The Ministry of Health, 1997).

TABLE 6
Cytotoxicity Evaluation Standard

| Toxicity Score | Level 0 | Level 1 | Level 2 | Level 3 | Level 4 | Level 5 |
|---|---|---|---|---|---|---|
| Relative | ≥100 | 75-99 | 50-74 | 25-49 | 1-24 | 0 |
| Growth Rate | | | | | | |

c. Statistical Analysis

SPSS 17.0 was used to carry out one-way analysis of variance on the absorbance values measured in the CCK-8 experiment. A q test method was adopted to carry out data analysis on the sample group and the blank group. When q was less than 0.05, it is indicated that two samples had a difference; when q was less than 0.01, it is indicated that the two samples had a significant difference; but when q was greater than 0.05, it is indicated that the two samples had no difference.

(2) Cytofluorescent Experiment

\textcircled{1} Principle of Cytofluorescent Experiment

ReadyProbes® Cell Viability Imaging Kit (Blue/Green) comprises NucBlue® Live reagent and NucGreen® Dead reagent. The NucBlue® Live reagent dyes the nucleus of living cells, while the NucGreen® Dead reagent only dyes the nucleus of cells with integrity damaged. The nucleus of living cells are blue, while the nucleus of dead cells are green.

\textcircled{2} Steps of Cytofluorescent Experiment

After cells in 96-well plates were cultured in an incubator for a corresponding time, all the original medium in the 96-well plates was sucked, and 200 μL of cell complete medium-fluorescent dye mixture (the preparation proportion of the complete medium-fluorescent dye mixture was that two drops of dye A and two drops of dye B were respectively added into every milliliter of cell complete medium and uniformly mixed before use) was added, with bubbles prevented from being produced to affect absorbance values. After addition, the 96-well plates were put into the incubator for 25 minutes of incubation. An inverted microscope was then used to observe the growth state of the cells in each group of samples.

(1) Results of Cytotoxicity Experiment on Hyaluronic Acid Hydrogels

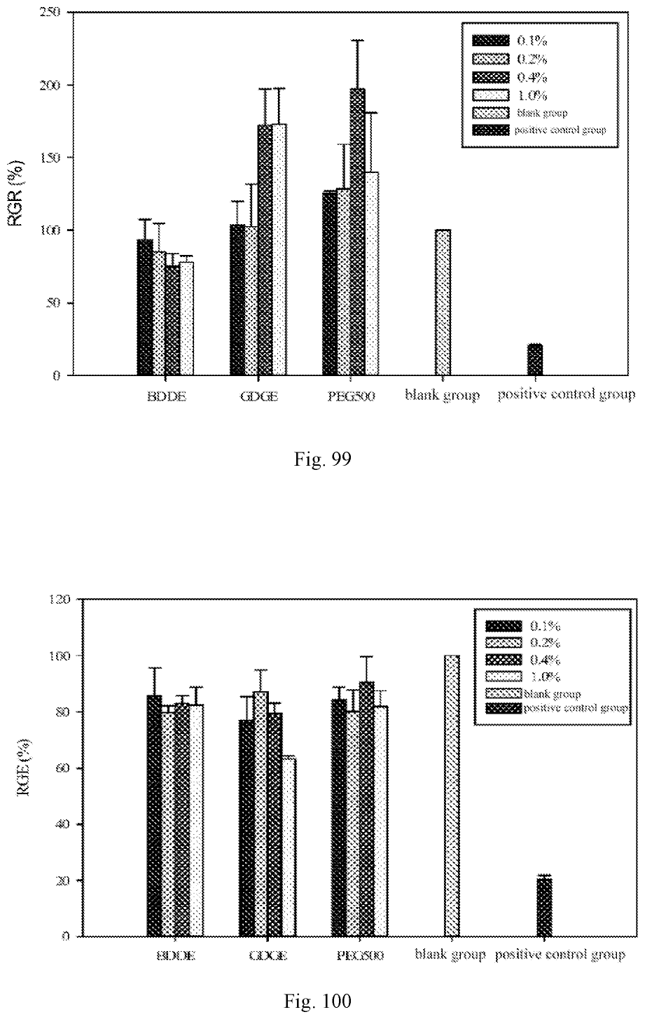

The cytotoxicity of the hydrogels was assayed, and the results were discussed and analyzed. L929 cells cultured normally at the same time were used as a blank control, L929 cells cultured with 0.64% phenol at the same time were chosen as a positive control, and the relative growth rates of the cells of the sample group were calculated. FIG. 99 is relative growth rates of cells after HA with a molecular weight of 1.5×10⁶ Da and different types of crosslinking agents at different crosslinking agent doses carried out crosslinking reaction under the condition of freeze-drying to obtain aerogels and hydrogels obtained by soaking the aerogels in PBS buffer solution were respectively cultured in an incubator for 2 days. It can be concluded from this results that when used as medical dressings, the hydrogels do not have cytotoxicity but need to be replaced regularly. The hydrogels prepared in this study can create a wet closed environment for wounds, accelerating wound healing. Moreover, these hydrogels can be used as temporary cell delivery carriers for regeneration of various tissues. Furthermore, the three-dimensional network structure of the hydrogels makes for the diffusion of oxygen and nutrient substances, creating a good environment for cells to support the survival and proliferation of the cells.

FIG. 100 shows relative growth rates of cells after HA with a molecular weight of 3.5×10⁵ Da and different types of crosslinking agents at different crosslinking agent doses carried out crosslinking reaction under the condition of freeze-drying to obtain aerogels and hydrogels obtained by soaking the aerogels in PBS buffer solution were respectively cultured in an incubator for 2 days. It can be concluded from this results that when used as medical dressings, the hydrogels do not have cytotoxicity but need to be replaced regularly. These hydrogels can create a wet closed environment for wounds, accelerating wound healing. Moreover, this hydrogel can be used as a temporary cell delivery carrier for regeneration of various tissues. Furthermore, the three-dimensional network structure of the hydrogel makes for the diffusion of oxygen and nutrient substances, providing a good environment for cells to support the survival and proliferation of the cells.

TABLE 7
Results of Cytotoxicity Experiment (After 2 days of cell culture)

| | | | | | 95% Confidence | | | |
|---|---|---|---|---|---|---|---|---|
| | | | Standard | Standard | Interval of Mean | | Minimal | Maximum |
| Sample Name | N | Mean | Deviation | Error | Upper Limit | Lower Limit | Value | Value |
| Blank | 3 | 0.47767 | 0.008622 | 0.004978 | 0.45625 | 0.49908 | 0.470 | 0.487 |
| HA³⁵-B-4-1 | 3 | 0.40900 | 0.047032 | 0.027154 | 0.29217 | 0.52583 | 0.363 | 0.457 |
| HA³⁵-B-4-2 | 3 | 0.38067 | 0.010970 | 0.006333 | 0.35342 | 0.40792 | 0.372 | 0.393 |
| HA³⁵-B-4-3 | 3 | 0.39600 | 0.013229 | 0.007638 | 0.36314 | 0.42886 | 0.386 | 0.411 |
| HA³⁵-B-4-4 | 3 | 0.39267 | 0.031214 | 0.018022 | 0.31513 | 0.47021 | 0.357 | 0.415 |
| HA³⁵-G-4-1 | 3 | 0.36700 | 0.039686 | 0.022913 | 0.26841 | 0.46559 | 0.337 | 0.412 |
| HA³⁵-G-4-2 | 3 | 0.41533 | 0.037207 | 0.021481 | 0.32291 | 0.50776 | 0.383 | 0.456 |
| HA³⁵-G-4-3 | 3 | 0.37967 | 0.017010 | 0.009821 | 0.33741 | 0.42192 | 0.367 | 0.399 |
| HA³⁵-G-4-4 | 3 | 0.30167 | 0.005686 | 0.003283 | 0.28754 | 0.31579 | 0.297 | 0.308 |
| HA³⁵-P500-4-1 | 3 | 0.40233 | 0.021548 | 0.012441 | 0.34880 | 0.45586 | 0.378 | 0.419 |
| HA³⁵-P500-4-2 | 3 | 0.38300 | 0.036290 | 0.020952 | 0.29285 | 0.47315 | 0.355 | 0.424 |
| HA³⁵-P500-4-3 | 3 | 0.43167 | 0.043662 | 0.025208 | 0.32321 | 0.54013 | 0.398 | 0.481 |
| HA³⁵-P500-4-4 | 3 | 0.39100 | 0.026665 | 0.015395 | 0.32476 | 0.45724 | 0.370 | 0.421 |
| HA¹⁵⁰-B-3-1 | 3 | 0.44600 | 0.067015 | 0.038691 | 0.27953 | 0.61247 | 0.371 | 0.500 |
| HA¹⁵⁰-B-3-2 | 3 | 0.40733 | 0.092916 | 0.053645 | 0.17652 | 0.63815 | 0.344 | 0.514 |
| HA¹⁵⁰-B-3-3 | 3 | 0.35867 | 0.042899 | 0.024768 | 0.25210 | 0.46523 | 0.310 | 0.391 |
| HA¹⁵⁰-B-3-4 | 3 | 0.37267 | 0.020404 | 0.011780 | 0.32198 | 0.42335 | 0.355 | 0.395 |
| HA¹⁵⁰-G-3-1 | 3 | 0.49500 | 0.078237 | 0.045170 | 0.30065 | 0.68935 | 0.410 | 0.564 |
| HA¹⁵⁰-G-3-2 | 3 | 0.48867 | 0.140429 | 0.081077 | 0.13982 | 0.83751 | 0.329 | 0.593 |
| HA¹⁵⁰-G-3-3 | 3 | 0.82267 | 0.119371 | 0.068919 | 0.52613 | 1.11920 | 0.692 | 0.926 |
| HA¹⁵⁰-G-3-4 | 3 | 0.82500 | 0.119025 | 0.068719 | 0.52932 | 1.12068 | 0.698 | 0.934 |
| HA¹⁵⁰-P500-3-1 | 3 | 0.59900 | 0.008544 | 0.004933 | 0.57778 | 0.62022 | 0.591 | 0.608 |
| HA¹⁵⁰-P500-3-2 | 3 | 0.61267 | 0.148075 | 0.085491 | 0.24483 | 0.98051 | 0.442 | 0.707 |
| HA¹⁵⁰-P500-3-3 | 3 | 0.94100 | 0.160137 | 0.092455 | 0.54320 | 1.33880 | 0.771 | 1.089 |
| HA¹⁵⁰-P500-3-4 | 3 | 0.66833 | 0.195439 | 0.112837 | 0.18284 | 1.15383 | 0.449 | 0.824 |
| Positive Control | 3 | 0.09733 | 0.006028 | 0.003480 | 0.08236 | 0.11231 | 0.091 | 0.103 |
| Sum | 78 | 0.47162 | 0.189229 | 0.021426 | 0.42895 | 0.51428 | 0.091 | 1.089 |

(2) Cytofluorescent Experiment on Hydrogels

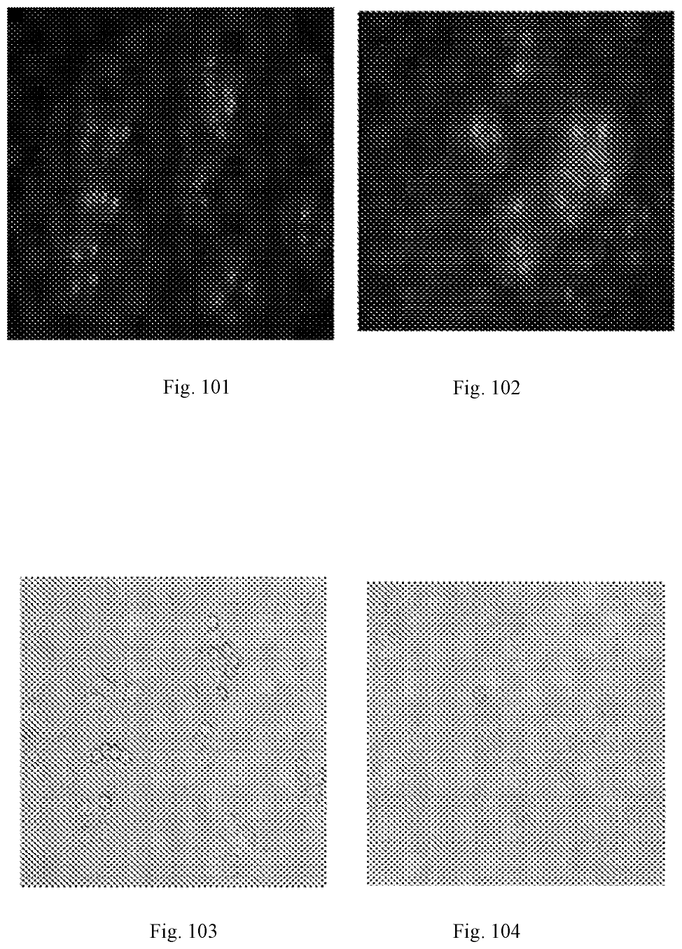

FIG. 101 and FIG. 102 are cytofluorescent images after the “HA³⁵-P500-4-3” sample and the “HA¹⁵⁰-P500-3-3” sample were respectively cultured for 2 days.

It can be known from the fluorescent images (FIG. 101 and FIG. 102) of co-culture of the L929 mouse fibroblasts and the hydrogels that as the culture time goes by, the growth density of the L929 cells is gradually increased. The results of the cytofluorescent experiment are similar to that of the CCK-8 experiment. As the hydrogels have the three-dimensional network structure, the diffusion of oxygen and nutrient substances can be promoted, which is beneficial for the survival and proliferation of cells, and this indicates that the hydrogels prepared in the present invention have good biocompatibility. It can be seen from the cytofluorescent images and the relative growth rates that these hydrogels can be used as cell delivery carriers to provide a good environment for cells to support the survival and proliferation of the cells, and can also be used as medical dressings to provide a wet closed environment for wounds, accelerating wound healing.

It can be seen by observing FIG. 103 and FIG. 104 that gel microstructure, appropriate cell inoculation density and sufficient nutrient substances in medium are important factors affecting the survival and proliferation of cells. The hydrogels prepared in the present invention can be used as cell delivery carriers to provide a good environment for cells to support the survival and proliferation of the cells, and can also be used as medical dressings to provide a wet closed environment for wounds, accelerating wound healing.

1: A preparation method of a covalently cross-linked hyaluronic acid aerogel and a hydrogel thereof, wherein a crosslinking agent is added into a hyaluronic acid aqueous solution to prepare a crosslinking reaction system; the crosslinking reaction system is subjected to crosslinking reaction in a freeze-drying environment immediately to prepare a covalently cross-linked hyaluronic acid aerogel; the obtained covalently cross-linked hyaluronic acid aerogel absorbs water and swells to obtain a covalently cross-linked hyaluronic acid hydrogel.

2: The preparation method of a covalently cross-linked hyaluronic acid aerogel and a hydrogel thereof according to claim 1, wherein the crosslinking agent is a diglycidyl ether crosslinking agent.

3: The preparation method of a covalently cross-linked hyaluronic acid aerogel and a hydrogel thereof according to claim 1, wherein the diglycidyl ether crosslinking agent is 1,4-butanediol diglycidyl ether, ethylene glycol diglycidyl ether or poly ethylene glycol diglycidyl ether (n=10, molecular weight: 500).

4: The preparation method of a covalently cross-linked hyaluronic acid aerogel and a hydrogel thereof according to claim 1, wherein the concentration of the crosslinking agent is 0.1% to 1% (v/v).

5: The preparation method of a covalently cross-linked hyaluronic acid aerogel and a hydrogel thereof according to claim 1, wherein the crosslinking reaction system reacts under a neutral condition of pH 6 to 8, preferably about 7.

6: The preparation method of a covalently cross-linked hyaluronic acid aerogel and a hydrogel thereof according to claim 1, wherein the molecular weight of hyaluronic acid is 3.5×10⁵ to 1.5×10⁶ Da.

7: The preparation method of a covalently cross-linked hyaluronic acid aerogel and a hydrogel thereof according to claim 1, wherein the concentration of the hyaluronic acid aqueous solution is 0.5% to 4% (w/v).

8: A covalently cross-linked hyaluronic acid aerogel and a hydrogel thereof prepared by the method according to claim 1.

9: A use of the covalently cross-linked hyaluronic acid aerogel and the hydrogel thereof according to claim 7 in cell engineering, tissue engineering, drug carriers or cosmetic surgery.

10: The use according to claim 9, wherein as surgical sealant and adhesive or wound dressing to promote wound healing.
